SEQUENCE LISTING (35 sequences)

SEQ ID NO: 1 - DNA, 729 bp, Artificial sequence
  Synthesized polynucleotide
gaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgt

SEQ ID NO: 2 - DNA, 63 bp, Artificial sequence
  Synthesized polynucleotide
atggccttaccagtgaccgccttgctcctgccgctggccttgctgctccacgccgccaggccg

SEQ ID NO: 3 - DNA, 135 bp, Artificial sequence
  Synthesized polynucleotide
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 4 - DNA, 123 bp, Artificial sequence
  Synthesized polynucleotide
aggagtaagaggagcaggctcctgcacagtgactacatgaacatgactccccgccgccccgggccaacccgcaagcattaccagccctatgccccaccacgcgacttcgcagcctatcgctcc

SEQ ID NO: 5 - DNA, 339 bp, Artificial sequence
  Synthesized polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - DNA, 81 bp, Artificial sequence
  Synthesized polynucleotide
ttttgggtgctggtggtggttggtggagtcctggcttgctatagcttgctagtaacagtggcctttattattttctgggtg

SEQ ID NO: 7 - DNA, 194 bp, Artificial sequence
  Synthesized polynucleotide
ggaggaaaaactgtttcatacagaaggcgtggaggaaaaactgtttcatacagaaggcgtggaggaaaaactgtttcatacagaaggcgtcaattgtcctcgacggaggaaaaactgtttcatacagaaggcgtggaggaaaaactgtttcatacagaaggcgtggaggaaaaactgtttcatacagaaggcgt

SEQ ID NO: 8 - DNA, 114 bp, Artificial sequence
  Synthesized polynucleotide
acattttgacacccccataatatttttccagaattaacagtataaattgcatctcttgttcaagagttccctatcactctctttaatcactactcacagtaacctcaactcctg

SEQ ID NO: 9 - DNA, 45 bp, Artificial sequence
  Synthesized polynucleotide
ggtggaggcggttcaggcggaggtggttctggcggtggcggatcg

SEQ ID NO: 10 - DNA, 984 bp, Artificial sequence
  Synthesized polynucleotide
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 11 - DNA, 594 bp, Artificial sequence
  Synthesized polynucleotide
agaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 12 - DNA, 81 bp, Artificial sequence
  Synthesized polynucleotide
atggcctcaccgttgacccgctttctgtcgctgaacctgctgctgctgggtgagtcgattatcctggggagtggagaagct

SEQ ID NO: 13 - DNA, 216 bp, Artificial sequence
  Synthesized polynucleotide
actactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccga

SEQ ID NO: 14 - DNA, 123 bp, Artificial sequence
  Synthesized polynucleotide
aatagtagaaggaacagactccttcaaagtgactacatgaacatgactccccggaggcctgggctcactcgaaagccttaccagccctacgcccctgccagagactttgcagcgtaccgcccc

SEQ ID NO: 15 - DNA, 321 bp, Artificial sequence
  Synthesized polynucleotide
agcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 16 - DNA, 192 bp, Artificial sequence
  Synthesized polynucleotide
aagaggaaaatttgtttcatacagaaggcgttaagaggaaaatttgtttcatacagaaggcgttaagaggaaaatttgtttcatacagaaggcgttaagaggaaaatttgtttcatacagaaggcgttaagaggaaaatttgtttcatacagaaggcgttaagaggaaaatttgtttcatacagaaggcgtt

SEQ ID NO: 17 - DNA, 114 bp, Artificial sequence
  Synthesized polynucleotide
aacatcgtgacacccccatattatttttccagcattaacagtataaattgcctcccatgctgaagagctgcctatcacccttgctaatcactcctcacagtgacctcaagtcct

SEQ ID NO: 18 - DNA, 1005 bp, Artificial sequence
  Synthesized polynucleotide
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcc

SEQ ID NO: 19 - DNA, 579 bp, Artificial sequence
  Synthesized polynucleotide
agggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 20 - DNA, 51 bp, Artificial sequence
  Synthesized polynucleotide
aataaaatatctttattttcattacatctgtgtgttggttttttgtgtgag

SEQ ID NO: 21 - protein, 469 aa, Artificial sequence
  Synthesized polypeptide
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 467 aa, Artificial sequence
  Synthesized polypeptide
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 511 aa, Artificial sequence
  Synthesized polypeptide
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 356 aa, Artificial sequence
  Synthesized polypeptide
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 243 aa, Artificial sequence
  Synthesized polypeptide
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 26 - protein, 540 aa, Artificial sequence
  Synthesized polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 27 - protein, 543 aa, Artificial sequence
  Synthesized polypeptide
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 28 - DNA, 4415 bp, Artificial sequence
  Synthesized polynucleotide
cgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtcctagcgctaccggtcgccaccatggccttaccagtgaccgccttgctcctgccgctggccttgctgctccacgccgccaggccggaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgctagcctcgagctcacacaaaaaaccaacacacagatgtaatgaaaataaagatattttattgcggccgctttaggaagcattcagatagctcatcactctatcaatagtcactgcccgaattctgaaagcatgaagaagtatgcagagcttgattttagttttataaaaatccggttcttcaagggaggatttttgtggcacagtctcactgttgaaattcagggcctgcatcagctcatcaataactgccagcatgttttgatctagaaagatctgcctcttaggatccatcagaagctttgcattcatggtcttgaactccacctggtacatcttcaagtcttcataaatactactaaggcacagggccatcataaaagaggtctttctggaggccaggcaactcccattagttatgaaagaggtctctctggaatttaggcaactctcattcttggttaattccaatggtaaacaggcctccactgtgctggttttatcttttgtgatatcttcatgatcaatctcttcagaagtgcaagggtaaaattctagagtttgtctggccttctggagcatgttgctgacggccctcagcaggttttgggagtggtgaaggcatgggaacattcctgggtctggagtggccacggggaggtttctagatccgccgccacccgacccaccaccgcccgagccaccgccaccactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcgaccggtagcgctaggtcatatgcaggagttgaggttactgtgagtagtgattaaagagagtgatagggaactcttgaacaagagatgcaatttatactgttaattctggaaaaatattatgggggtgtcaaaatgtcccgggacaattgacgccttctgtatgaaacagtttttcctccacgccttctgtatgaaacagtttttcctccacgccttctgtatgaaacagtttttcctccgtcgaggacaattgacgccttctgtatgaaacagtttttcctccacgccttctgtatgaaacagtttttcctccacgccttctgtatgaaacagtttttcctcc

SEQ ID NO: 29 - DNA, 4418 bp, Artificial sequence
  Synthesized polynucleotide
cgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtcctagcgctaccggtcgccaccatggccttaccagtgaccgccttgctcctgccgctggccttgctgctccacgccgccaggccggaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgctagcctcgagctcacacaaaaaaccaacacacagatgtaatgaaaataaagatattttattgcggccgctttaggaagcattcagatagctcatcactctatcaatagtcactgcccgaattctgaaagcatgaagaagtatgcagagcttgattttagttttataaaaatccggttcttcaagggaggatttttgtggcacagtctcactgttgaaattcagggcctgcatcagctcatcaataactgccagcatgttttgatctagaaagatctgcctcttaggatccatcagaagctttgcattcatggtcttgaactccacctggtacatcttcaagtcttcataaatactactaaggcacagggccatcataaaagaggtctttctggaggccaggcaactcccattagttatgaaagaggtctctctggaatttaggcaactctcattcttggttaattccaatggtaaacaggcctccactgtgctggttttatcttttgtgatatcttcatgatcaatctcttcagaagtgcaagggtaaaattctagagtttgtctggccttctggagcatgttgctgacggccctcagcaggttttgggagtggtgaaggcatgggaacattcctgggtctggagtggccacggggaggtttctagatccgccgccacccgacccaccaccgcccgagccaccgccaccactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcgaccggtagcgctaggtcatatgcaggagttgaggttactgtgagtagtgattaaagagagtgatagggaactcttgaacaagagatgcaatttatactgttaattctggaaaaatattatgggggtgtcaaaatgtcccgggacaattgacgccttctgtatgaaacagtttttcctccacgccttctgtatgaaacagtttttcctccacgccttctgtatgaaacagtttttcctccgtcgaggacaattgacgccttctgtatgaaacagtttttcctccacgccttctgtatgaaacagtttttcctccacgccttctgtatgaaacagtttttcctcc

SEQ ID NO: 30 - DNA, 4541 bp, Artificial sequence
  Synthesized polynucleotide
cgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtcctagcgctaccggtcgccaccatggccttaccagtgaccgccttgctcctgccgctggccttgctgctccacgccgccaggccggaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgctagcctcgagctcacacaaaaaaccaacacacagatgtaatgaaaataaagatattttattgcggccgctttaggaagcattcagatagctcatcactctatcaatagtcactgcccgaattctgaaagcatgaagaagtatgcagagcttgattttagttttataaaaatccggttcttcaagggaggatttttgtggcacagtctcactgttgaaattcagggcctgcatcagctcatcaataactgccagcatgttttgatctagaaagatctgcctcttaggatccatcagaagctttgcattcatggtcttgaactccacctggtacatcttcaagtcttcataaatactactaaggcacagggccatcataaaagaggtctttctggaggccaggcaactcccattagttatgaaagaggtctctctggaatttaggcaactctcattcttggttaattccaatggtaaacaggcctccactgtgctggttttatcttttgtgatatcttcatgatcaatctcttcagaagtgcaagggtaaaattctagagtttgtctggccttctggagcatgttgctgacggccctcagcaggttttgggagtggtgaaggcatgggaacattcctgggtctggagtggccacggggaggtttctagatccgccgccacccgacccaccaccgcccgagccaccgccaccactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcgaccggtagcgctaggtcatatgcaggagttgaggttactgtgagtagtgattaaagagagtgatagggaactcttgaacaagagatgcaatttatactgttaattctggaaaaatattatgggggtgtcaaaatgtcccgggacaattgacgccttctgtatgaaacagtttttcctccacgccttctgtatgaaacagtttttcctccacgccttctgtatgaaacagtttttcctccgtcgaggacaattgacgccttctgtatgaaacagtttttcctccacgccttctgtatgaaacagtttttcctccacgccttctgtatgaaacagtttttcctcc

SEQ ID NO: 31 - DNA, 4292 bp, Artificial sequence
  Synthesized polynucleotide
cgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtcctagcgctaccggtcgccaccatggccttaccagtgaccgccttgctcctgccgctggccttgctgctccacgccgccaggccggaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgctagcctcgagctcacacaaaaaaccaacacacagatgtaatgaaaataaagatattttattgcggccgctttaggaagcattcagatagctcatcactctatcaatagtcactgcccgaattctgaaagcatgaagaagtatgcagagcttgattttagttttataaaaatccggttcttcaagggaggatttttgtggcacagtctcactgttgaaattcagggcctgcatcagctcatcaataactgccagcatgttttgatctagaaagatctgcctcttaggatccatcagaagctttgcattcatggtcttgaactccacctggtacatcttcaagtcttcataaatactactaaggcacagggccatcataaaagaggtctttctggaggccaggcaactcccattagttatgaaagaggtctctctggaatttaggcaactctcattcttggttaattccaatggtaaacaggcctccactgtgctggttttatcttttgtgatatcttcatgatcaatctcttcagaagtgcaagggtaaaattctagagtttgtctggccttctggagcatgttgctgacggccctcagcaggttttgggagtggtgaaggcatgggaacattcctgggtctggagtggccacggggaggtttctagatccgccgccacccgacccaccaccgcccgagccaccgccaccactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcgaccggtagcgctaggtcatatgcaggagttgaggttactgtgagtagtgattaaagagagtgatagggaactcttgaacaagagatgcaatttatactgttaattctggaaaaatattatgggggtgtcaaaatgtcccgggacaattgacgccttctgtatgaaacagtttttcctccacgccttctgtatgaaacagtttttcctccacgccttctgtatgaaacagtttttcctccgtcgaggacaattgacgccttctgtatgaaacagtttttcctccacgccttctgtatgaaacagtttttcctccacgccttctgtatgaaacagtttttcctcc

SEQ ID NO: 32 - DNA, 3391 bp, Artificial sequence
  Synthesized polynucleotide
atggcctcaccgttgacccgctttctgtcgctgaacctgctgctgctgggtgagtcgattatcctggggagtggagaagctgaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcctaggtcgactcacacaaaaaaccaacacacagatgtaatgaaaataaagatattttattcgtacgttaggcggagctcagatagcccatcaccctgttgatggtcacgacgcgggtgctgaaggcgtgaagcaggatgcagagcttcattttcactctgtaagggtctgcttctcccacaggaggtttctggcgcagagtctcgccattatgattcagagactgcatcagctcatcgatggccaccagcatgcccttgtctagaatgatctgctgatggttgtgattctgaagtgctgcgttgatggcctggaactctgtctggtacatcttcaagtcctcatagatgctaccaaggcacagggtcatcatcaaagacgtcttctgtgggggcaggcagctccctcttgttgtggaagaagtctctctagtagccaggcaactctcgttcttgtgtagttccagtggtaaacaggtcttcaatgtgctggtttggtcccgtgtgatgtcttcatgatcgatgtcttcagcagtgcaggaataatgtttcagtttttctctggccgtcttcaccatgtcatctgtggtcttcagcaggtttcgggactggctaagacacctggcaggtccagagactggaatgaccctagatccgccgccacccgacccaccaccgcccgagccaccgccaccggatcggaccctgcagggaacacatgcccacttgctgcatgaggaattgtaatagcgatcctgagcttgcacgcagacattcccgcctttgcattggacttcggtagatgtcttctctacgaggaacgcacctttctggttacacccctcctctgtctccttcatcttttctttcttgcgctggattcgaacaaagaacttgagggagaagtaggaatggggagtgctccaggagtcagggtactcccagctgacctccacctgtgagttcttcaaaggcttcatctgcaagttcttgggcgggtctggtttgatgatgtccctgatgaagaagctggtgctgtagttctcatatttattctgctgccgtgcttccaacgccagttcaatgggcagggtctcctcggcagttgggcaggtgacatcctcctggcaggacactgaatacttctcatagtccctttggtccagtgtgaccttctctgcagacagagacgccattccacatgtcactgcccgagagtcaggggaactgctactgctcttgatgttgaacttcaagtccatgtttctttgcaccagccatgagcacgtgaaccgtccggagtaatttggtgcttcacacttcaggaaagtcttgtttttgaaattttttaaaatttcagtggaccaaattccattttccttcttgtggagcagcagatgtgagtggctcagagtctcgcctcctttgtggcaggtgtactggccagcatctagaaactctttgacagtgatggtcagggtctttccagagcctatgactccatgtctctggtctgaggtccaggtgatgtcatcttcttcaggcgtgtcacaggtgaggttcactgtttctccaggggcatcgggagtccagtccacctctacaacataaacgtctttctccagctcccacatggccatgagtggagacaccagcaaaacgatggcaaaccaggagatggttagcttctgaggacacatggtggcgaccggtagcgctaggacgcgtaggacttgaggtcactgtgaggagtgattagcaagggtgataggcagctcttcagcatgggaggcaatttatactgttaatgctggaaaaataatatgggggtgtcacgatgttcccgggacaattgaacgccttctgtatgaaacaaattttcctcttaacgccttctgtatgaaacaaattttcctcttaacgccttctgtatgaaacaaattttcctcttaacgccttctgtatgaaacaaattttcctcttaacgccttctgtatgaaacaaattttcctcttaacgccttctgtatgaaacaaattttcctctt

SEQ ID NO: 33 - DNA, 3514 bp, Artificial sequence
  Synthesized polynucleotide
atggcctcaccgttgacccgctttctgtcgctgaacctgctgctgctgggtgagtcgattatcctggggagtggagaagctgaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaatagtagaaggaacagactccttcaaagtgactacatgaacatgactccccggaggcctgggctcactcgaaagccttaccagccctacgcccctgccagagactttgcagcgtaccgccccagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcctaggtcgactcacacaaaaaaccaacacacagatgtaatgaaaataaagatattttattcgtacgttaggcggagctcagatagcccatcaccctgttgatggtcacgacgcgggtgctgaaggcgtgaagcaggatgcagagcttcattttcactctgtaagggtctgcttctcccacaggaggtttctggcgcagagtctcgccattatgattcagagactgcatcagctcatcgatggccaccagcatgcccttgtctagaatgatctgctgatggttgtgattctgaagtgctgcgttgatggcctggaactctgtctggtacatcttcaagtcctcatagatgctaccaaggcacagggtcatcatcaaagacgtcttctgtgggggcaggcagctccctcttgttgtggaagaagtctctctagtagccaggcaactctcgttcttgtgtagttccagtggtaaacaggtcttcaatgtgctggtttggtcccgtgtgatgtcttcatgatcgatgtcttcagcagtgcaggaataatgtttcagtttttctctggccgtcttcaccatgtcatctgtggtcttcagcaggtttcgggactggctaagacacctggcaggtccagagactggaatgaccctagatccgccgccacccgacccaccaccgcccgagccaccgccaccggatcggaccctgcagggaacacatgcccacttgctgcatgaggaattgtaatagcgatcctgagcttgcacgcagacattcccgcctttgcattggacttcggtagatgtcttctctacgaggaacgcacctttctggttacacccctcctctgtctccttcatcttttctttcttgcgctggattcgaacaaagaacttgagggagaagtaggaatggggagtgctccaggagtcagggtactcccagctgacctccacctgtgagttcttcaaaggcttcatctgcaagttcttgggcgggtctggtttgatgatgtccctgatgaagaagctggtgctgtagttctcatatttattctgctgccgtgcttccaacgccagttcaatgggcagggtctcctcggcagttgggcaggtgacatcctcctggcaggacactgaatacttctcatagtccctttggtccagtgtgaccttctctgcagacagagacgccattccacatgtcactgcccgagagtcaggggaactgctactgctcttgatgttgaacttcaagtccatgtttctttgcaccagccatgagcacgtgaaccgtccggagtaatttggtgcttcacacttcaggaaagtcttgtttttgaaattttttaaaatttcagtggaccaaattccattttccttcttgtggagcagcagatgtgagtggctcagagtctcgcctcctttgtggcaggtgtactggccagcatctagaaactctttgacagtgatggtcagggtctttccagagcctatgactccatgtctctggtctgaggtccaggtgatgtcatcttcttcaggcgtgtcacaggtgaggttcactgtttctccaggggcatcgggagtccagtccacctctacaacataaacgtctttctccagctcccacatggccatgagtggagacaccagcaaaacgatggcaaaccaggagatggttagcttctgaggacacatggtggcgaccggtagcgctaggacgcgtaggacttgaggtcactgtgaggagtgattagcaagggtgataggcagctcttcagcatgggaggcaatttatactgttaatgctggaaaaataatatgggggtgtcacgatgttcccgggacaattgaacgccttctgtatgaaacaaattttcctcttaacgccttctgtatgaaacaaattttcctcttaacgccttctgtatgaaacaaattttcctcttaacgccttctgtatgaaacaaattttcctcttaacgccttctgtatgaaacaaattttcctcttaacgccttctgtatgaaacaaattttcctctt

SEQ ID NO: 34 - DNA, 3526 bp, Artificial sequence
  Synthesized polynucleotide
atggcctcaccgttgacccgctttctgtcgctgaacctgctgctgctgggtgagtcgattatcctggggagtggagaagctgaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcctaggtcgactcacacaaaaaaccaacacacagatgtaatgaaaataaagatattttattcgtacgttaggcggagctcagatagcccatcaccctgttgatggtcacgacgcgggtgctgaaggcgtgaagcaggatgcagagcttcattttcactctgtaagggtctgcttctcccacaggaggtttctggcgcagagtctcgccattatgattcagagactgcatcagctcatcgatggccaccagcatgcccttgtctagaatgatctgctgatggttgtgattctgaagtgctgcgttgatggcctggaactctgtctggtacatcttcaagtcctcatagatgctaccaaggcacagggtcatcatcaaagacgtcttctgtgggggcaggcagctccctcttgttgtggaagaagtctctctagtagccaggcaactctcgttcttgtgtagttccagtggtaaacaggtcttcaatgtgctggtttggtcccgtgtgatgtcttcatgatcgatgtcttcagcagtgcaggaataatgtttcagtttttctctggccgtcttcaccatgtcatctgtggtcttcagcaggtttcgggactggctaagacacctggcaggtccagagactggaatgaccctagatccgccgccacccgacccaccaccgcccgagccaccgccaccggatcggaccctgcagggaacacatgcccacttgctgcatgaggaattgtaatagcgatcctgagcttgcacgcagacattcccgcctttgcattggacttcggtagatgtcttctctacgaggaacgcacctttctggttacacccctcctctgtctccttcatcttttctttcttgcgctggattcgaacaaagaacttgagggagaagtaggaatggggagtgctccaggagtcagggtactcccagctgacctccacctgtgagttcttcaaaggcttcatctgcaagttcttgggcgggtctggtttgatgatgtccctgatgaagaagctggtgctgtagttctcatatttattctgctgccgtgcttccaacgccagttcaatgggcagggtctcctcggcagttgggcaggtgacatcctcctggcaggacactgaatacttctcatagtccctttggtccagtgtgaccttctctgcagacagagacgccattccacatgtcactgcccgagagtcaggggaactgctactgctcttgatgttgaacttcaagtccatgtttctttgcaccagccatgagcacgtgaaccgtccggagtaatttggtgcttcacacttcaggaaagtcttgtttttgaaattttttaaaatttcagtggaccaaattccattttccttcttgtggagcagcagatgtgagtggctcagagtctcgcctcctttgtggcaggtgtactggccagcatctagaaactctttgacagtgatggtcagggtctttccagagcctatgactccatgtctctggtctgaggtccaggtgatgtcatcttcttcaggcgtgtcacaggtgaggttcactgtttctccaggggcatcgggagtccagtccacctctacaacataaacgtctttctccagctcccacatggccatgagtggagacaccagcaaaacgatggcaaaccaggagatggttagcttctgaggacacatggtggcgaccggtagcgctaggacgcgtaggacttgaggtcactgtgaggagtgattagcaagggtgataggcagctcttcagcatgggaggcaatttatactgttaatgctggaaaaataatatgggggtgtcacgatgttcccgggacaattgaacgccttctgtatgaaacaaattttcctcttaacgccttctgtatgaaacaaattttcctcttaacgccttctgtatgaaacaaattttcctcttaacgccttctgtatgaaacaaattttcctcttaacgccttctgtatgaaacaaattttcctcttaacgccttctgtatgaaacaaattttcctctt

SEQ ID NO: 35 - DNA, 3649 bp, Artificial sequence
  Synthesized polynucleotide
atggcctcaccgttgacccgctttctgtcgctgaacctgctgctgctgggtgagtcgattatcctggggagtggagaagctgaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaatagtagaaggaacagactccttcaaagtgactacatgaacatgactccccggaggcctgggctcactcgaaagccttaccagccctacgcccctgccagagactttgcagcgtaccgccccaaatggatcaggaaaaaattcccccacatattcaagcaaccatttaagaagaccactggagcagctcaagaggaagatgcttgtagctgccgatgtccacaggaagaagaaggaggaggaggaggctatgagctgagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcctaggtcgactcacacaaaaaaccaacacacagatgtaatgaaaataaagatattttattcgtacgttaggcggagctcagatagcccatcaccctgttgatggtcacgacgcgggtgctgaaggcgtgaagcaggatgcagagcttcattttcactctgtaagggtctgcttctcccacaggaggtttctggcgcagagtctcgccattatgattcagagactgcatcagctcatcgatggccaccagcatgcccttgtctagaatgatctgctgatggttgtgattctgaagtgctgcgttgatggcctggaactctgtctggtacatcttcaagtcctcatagatgctaccaaggcacagggtcatcatcaaagacgtcttctgtgggggcaggcagctccctcttgttgtggaagaagtctctctagtagccaggcaactctcgttcttgtgtagttccagtggtaaacaggtcttcaatgtgctggtttggtcccgtgtgatgtcttcatgatcgatgtcttcagcagtgcaggaataatgtttcagtttttctctggccgtcttcaccatgtcatctgtggtcttcagcaggtttcgggactggctaagacacctggcaggtccagagactggaatgaccctagatccgccgccacccgacccaccaccgcccgagccaccgccaccggatcggaccctgcagggaacacatgcccacttgctgcatgaggaattgtaatagcgatcctgagcttgcacgcagacattcccgcctttgcattggacttcggtagatgtcttctctacgaggaacgcacctttctggttacacccctcctctgtctccttcatcttttctttcttgcgctggattcgaacaaagaacttgagggagaagtaggaatggggagtgctccaggagtcagggtactcccagctgacctccacctgtgagttcttcaaaggcttcatctgcaagttcttgggcgggtctggtttgatgatgtccctgatgaagaagctggtgctgtagttctcatatttattctgctgccgtgcttccaacgccagttcaatgggcagggtctcctcggcagttgggcaggtgacatcctcctggcaggacactgaatacttctcatagtccctttggtccagtgtgaccttctctgcagacagagacgccattccacatgtcactgcccgagagtcaggggaactgctactgctcttgatgttgaacttcaagtccatgtttctttgcaccagccatgagcacgtgaaccgtccggagtaatttggtgcttcacacttcaggaaagtcttgtttttgaaattttttaaaatttcagtggaccaaattccattttccttcttgtggagcagcagatgtgagtggctcagagtctcgcctcctttgtggcaggtgtactggccagcatctagaaactctttgacagtgatggtcagggtctttccagagcctatgactccatgtctctggtctgaggtccaggtgatgtcatcttcttcaggcgtgtcacaggtgaggttcactgtttctccaggggcatcgggagtccagtccacctctacaacataaacgtctttctccagctcccacatggccatgagtggagacaccagcaaaacgatggcaaaccaggagatggttagcttctgaggacacatggtggcgaccggtagcgctaggacgcgtaggacttgaggtcactgtgaggagtgattagcaagggtgataggcagctcttcagcatgggaggcaatttatactgttaatgctggaaaaataatatgggggtgtcacgatgttcccgggacaattgaacgccttctgtatgaaacaaattttcctcttaacgccttctgtatgaaacaaattttcctcttaacgccttctgtatgaaacaaattttcctcttaacgccttctgtatgaaacaaattttcctcttaacgccttctgtatgaaacaaattttcctcttaacgccttctgtatgaaacaaattttcctctt

(19) United States
(12) Patent Application Publication
(10) Pub. No.: US 2022/0056408 A1
(43) Pub. Date: Feb. 24, 2022

(54) IMMUNE EFFECTOR CELL TARGETING GPC3 AND APPLICATION THEREOF

(71) Applicants: CARSGEN THERAPEUTICS CO., LTD., Shanghai (CN); SHANGHAI CANCER INSTITUTE, Shanghai (CN)

(72) Inventors: Zonghai LI, Shanghai (CN); Hua JIANG, Shanghai (CN)

(21) Appl. No.: 17/413,892

(22) PCT Filed: Dec. 13, 2018
(86) PCT No.: PCT/CN2018/120938

§ 371 Date: Jun. 14, 2021

(51) Int. Cl. C12N 5/0783; C12N 5/10; C12N 15/86; A61P 35/00; A61K 35/17; C07K 14/705; C07K 14/54; C07K 14/725; C07K 16/30

(52) U.S. Cl. CPC C12N 5/0636; A61K 38/00; C12N 15/86; A61P 35/00; A61K 35/17; C07K 14/705; C07K 14/5434; C07K 14/70521; C07K 14/7051; C07K 16/303; C07K 14/70517; C12N 2510/00; C07K 2317/622; C07K 2317/76; C12N 5/10

(57) ABSTRACT

The present invention relates to an immune effector cell which expresses a chimeric antigen receptor targeting GPC3 and exogenous IL12. The present invention further provides a pharmaceutical composition containing the immune effector cell and a method for treating tumor, particularly GPC3 positive tumor by using the immune effector cell or the pharmaceutical composition. The immune effector cell in the present invention is effective to an entity tumor cell in vitro, and is outstanding in effect of extinguishing the entity tumor cell in vivo.

### TECHNICAL FIELD

The present invention relates to the field of cellular immunotherapy, and in particular, to a genetically modified immune effector cell targeting GPC3.

### BACKGROUND

Chimeric antigen receptor engineered T lymphocyte (CAR-T) technology is a new strategy for tumor biotherapy that has emerged in recent years. Chimeric antigen receptor (CAR) combines a single-chain antibody recognizing a tumor-associated antigen and an activating motif of immune cells, and T lymphocytes can express chimeric antigen receptors in vitro by means of genetic engineering, thereby rendering T cells the ability to specifically recognize and kill tumor cells. At present, good results in the treatment of hematological malignancies have been achieved by CAR-T. For example, great success has been achieved in patients of CD19-positive B lymphocytic leukemia. However, CAR-T is still faced with great challenges in the treatment of solid tumors.

Interleukin-12 (IL12) is an important immunostimulatory cytokine that can promote the proliferation of helper T cells and induce NK cells and T cells to produce interferon-gamma. However, in clinical trials, it was found that serious side effects occurred in multiple organs during systemic administration of IL12, such as abnormal liver function, high fever, severe hemodynamic instability, etc. The severe cases resulted in death, so that the application of IL12 is restricted.

In addition, due to the particularity of tumors, especially solid tumors, the microenvironment of each tumor is completely different. It is unknown whether immune effector cells co-expressing IL12 can have enhanced anti-tumor effects, especially the significantly enhanced anti-tumor effects, and certain side effects will be caused, such as weight loss of experimental mice. In addition, the combination of CAR T cells and other cytokines also exhibits disadvantages, such as great side effects or poor in vivo effects. Therefore, effects produced by specific chimeric antigen receptor immune effector cells in combination with specific cytokines cannot be reasonably expected.

Therefore, there is an urgent need in the art for immune effector cells having significant anti-tumor effects but low side effects.

### SUMMARY OF THE INVENTION

The object of the present invention is to provide an immune effector cell with significant anti-tumor effects but low side effects.

In the first aspect, the present invention provides an immune effector cell expressing a receptor and exogenous IL12, wherein the receptor specifically binds to GPC3.

In a specific embodiment, the receptor and the IL12 are operably linked.

In a specific embodiment, the immune effector cells include: T cells, natural killer cells, cytotoxic T lymphocytes, natural killer T cells, DNT cells, and/or regulatory T cells.

In a specific embodiment, the exogenous IL12 is constitutively or inductively expressed; preferably, the promoter used to express the IL12 includes: an immune cell inducible promoter; and preferably, the immune cell inducible promoter is NFAT6 promoter.

In a specific embodiment, the receptor has an extracellular domain specifically binding to GPC3, a transmembrane domain, and an intracellular signal domain.

In a specific embodiment, the receptor is a chimeric antigen receptor, wherein the intracellular domain contains a cell stimulating signal molecule or a combination of a cell stimulating signal molecule and a cell activating co-stimulatory molecule;

Preferably, the cell stimulating signal molecule is selected from the functional signaling domain of a protein: CD3ζ, CD3γ, CD3δ, CD3ε, FcεRIγ, FcRβ, CD79a, CD79b, FcγRIIa, DAP10, or DAP12; more preferably CD3ζ; or

Preferably, the cell activating co-stimulatory molecule is selected from the functional signaling domain of the following proteins: CD27, CD28, CD137, CD134, ICOS, OX40, CD30, CD40, PD-1, lymphocyte function related antigen-1 (LFA-1), CD2, CD7, LIGHT, NKG2C, B7-H3, a ligand that specifically binds to CD83, CDS, ICAM-1, GITR, BAFFR, HVEM (LIGHTR), SLAMF7, NKp80 (KLRF1), CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, more preferably, CD27, CD28, CD137, CD134, ICOS.

In a specific embodiment, the extracellular domain of the receptor contains an amino acid sequence that is at least 90%, 95%, 96%, 97%, 98%, or 99% identical with the amino acid sequence shown in SEQ ID NO: 25.

In a specific embodiment, the receptor contains the amino acid sequence shown in SEQ ID NO: 21, 22, 23, or 24; or contains an amino acid sequence that is at least 90%, 95%, 96%, 97%, 98%, or 99% identical with the amino acid sequence shown in SEQ ID NO: 21, 22, 23 or 24.

In a specific embodiment, the receptor and the exogenous IL12 are encoded by a nucleotide sequence that is at least 90%, 95%, 96%, 97%, 98%, or 99% identity with SEQ ID NO: 27, 28, or 29.

In a specific embodiment, the immune effector cells do not contain exogenous co-stimulatory ligands.

In a specific embodiment, the receptor and/or the IL12 are constitutively or inducibly expressed on the surface of immune effector cells.

In a specific embodiment, the immune effector cell contains an expression construct, which includes: an expression cassette of the antigen-binding receptor; and an expression cassette of the IL12.

In a specific embodiment, the receptor and/or IL12 are expressed by using a viral vector; and preferably, the viral vector includes: a lentiviral vector, retroviral vector or adenoviral vector.

In a specific embodiment, after the immune effector cells are administered to an individual, the number of CAR-T cells in the peripheral blood of the individual is increased by at least 50%, compared with the case where the exogenous IL12 is not present.

In a specific embodiment, about 7 days after the immune effector cells are administered to the individual, the sum of the number of CAR-T cells in the individual's peripheral blood is greater than 6,000/μL; and about 10 days after the immune effector cells are administered, the sum of the number of CAR-T cells in the individual's peripheral blood is greater than 6,000/μL.

In a preferred embodiment, the immune effector cells of the present invention exhibit excellent killing effects on tumors that cannot be controlled by a low dose, such as a dose of less than 1*10⁷ cells/L of immune effector cells not of the present invention (that is, immune effector cells which do not express a receptor specifically binding to GPC3 and exogenous IL12 simultaneously), or tumors that are too large (such as tumors larger than 300 mm³ in the mouse tumor model, or even larger than 600 mm³).

In the second aspect, the present invention provides an expression construct, including an expression cassette of a receptor and an expression cassette of IL12, which are connected in sequence; wherein the antigen-binding receptor and IL12 are described as in the first aspect of the present invention.

In a third aspect, the present invention provides the use of the immune effector cells described in the first aspect of the present invention in the preparation of a pharmaceutical composition for treating tumors in an individual in need thereof.

In a specific embodiment, the tumor includes: liver cancer, stomach cancer, lung cancer, breast cancer, head and neck cancer, bladder cancer, ovarian cancer, cervical cancer, kidney cancer, pancreatic cancer, cervical cancer, liposarcoma, melanoma, adrenal carcinoma, Schwannoma, malignant fibrous histiocytoma, esophageal cancer; and preferably, the tumor is liver cancer, gastric cancer, lung cancer, or breast cancer.

In a specific embodiment, the immune effector cells reduce the tumor by at least 50%.

In a specific embodiment, the immune effector cells reduce the tumor by at least 70%, and more preferably, the immune effector cells reduce the tumor by at least 80%.

In the fourth aspect, the present invention provides a pharmaceutical composition, comprising:

the immune effector cell of the first aspect of the present invention; and

a pharmaceutically acceptable carrier or excipient.

In the fifth aspect, the present invention provides a kit comprising:

the immune effector cell of the first aspect of the present invention; and

instructions on how to administer the immune effector cell to an individual.

It should be understood that, within the scope of the present invention, the above-mentioned technical features of the present invention and the technical features specifically described in the following contents (such as the Examples) can be combined with each other to form a new or preferred technical solution, which will not be repeated one by one due to the limited contents herein.

### DESCRIPTION OF DRAWINGS

FIG. 1A is a schematic diagram of the structure of a plasmid prepared for CAR-T cells; and FIG. 1B shows the expression of CAR on the surface of GPC3-CAR-T/IL12-GPC3-CAR-T cells detected by FACS;

FIG. 2 shows the experimental results of in vitro toxicity of CAR-T cells;

FIG. 3A shows the secretion of IL-12 when CAR-T cells and tumor cells are co-incubated for 24 hours; and FIG. 3B shows the secretion of IL-2/TNF-α/IFN-γ when CAR-T cells and tumor cells are co-incubated for 24 hours;

FIG. 4A shows the growth of Huh-7 subcutaneously transplanted tumors in different experimental groups; and FIG. 4B shows changes in the weight of mice with Huh-7 subcutaneously transplanted tumors in different experimental groups;

FIG. 5 shows the number of surviving cells of different CAR-T in peripheral blood on day 7 and day 10;

FIG. 6 shows the amount of IFN-γ in serum samples on day 10 after the injection of CAR-T cells;

FIG. 7 shows the results of immunohistochemical detection of infiltrating T cells and Ki67 in tumor tissues;

FIGS. 8A and 8B show the growth of subcutaneous tumors in mice at the end of the experiment;

FIG. 9 shows the expression of CAR on the surface of mCAR-T/mIL12-GPC3-CAR T cells detected by FACS;

FIG. 10 shows the experimental results of in vitro toxicity of mCAR-T;

FIG. 11A shows the secretion of mIL-12 when UTD, mGPC3-CAR, mGPC3-m28Z-mNFAT6-mIL12 are incubated with tumor cells for 24 hours; and FIG. 11B shows the release of cytokines mIL-2, mTNF-α and mIFN-γ when UTD/mGPC3-m28Z/mIL12-GPC3-CAR T and tumor cells are co-incubated for 24 hours;

FIG. 12A and FIG. 12B show therapeutic effects on E0771-GPC3 subcutaneous transplantation tumor;

FIG. 13A shows the experimental results of the group, in which mIL12-GPC3-T 5×106/animal was injected through vein injection, and the group, in which mIL12-GPC3-T 2λ106/animal was injected through vein injection; and FIG. 13B shows the body weights of mice in mGPC3-CAR-T group and blank control group, after mIL12-GPC3-T were administered.

### MODES FOR CARRYING OUT THE INVENTION

After extensive and in-depth research, the inventors unexpectedly discovered that the expression of a receptor that specifically binds to GPC3 and exogenous IL12 in immune effector cells will have significant anti-tumor effects, but with low side effects, based on which the present invention has been completed.

The following detailed description shows the embodiments disclosed herein in detail. It should be understood that this specification is not intended to be limited to the specific embodiments disclosed herein, which may be changed. A skilled person in the art will understand that there may be various changes or modifications of the contents disclosed in this specification, all of which shall fall within the scope and principle of the disclosure. Unless otherwise specified, each embodiment can be arbitrarily combined with any other embodiment.

Certain embodiments disclosed herein include numerical ranges, and certain aspects of the present invention may be described in terms of ranges. Unless otherwise specified, it should be understood that a numerical range or the description of a range is provided only for brevity and convenience, while should not be considered as a strict limitation on the scope of the present invention. Therefore, the description of a range should be regarded as specifically disclosing all possible sub-ranges and all possible specific numerical points within the range, just as these sub-ranges and numerical points have been clearly written herein. For example, the description of a range from 1 to 6 should be considered as specifically disclosing sub-ranges from 1 to 3, 1 to 4, 1 to 5, 2 to 4, 2 to 6, 3 to 6, and the like, and specific numerical points within these ranges, such as 1, 2, 3, 4, 5, 6. Regardless of the width of the stated range, the above principles are equally applicable. When a range is described, the endpoints of the range are also included in the range.

### Terms

As used herein, the terms “activation” and “activating” can bee used interchangeably, and they and other grammatical forms can refer to a process by which a cell changes from a resting state to an active state. The process may include a response to phenotypic or genetic changes of antigen, migration, and/or functional activity status. For example, the term “activation” can refer to a process of gradual activation of T cells. For example, at least two signals may be required to fully activate T cells. The first signal can occur after the conjugation of TCR with antigen-MHC complex, and the second signal can occur through the conjugation of co-stimulatory molecules (see the costimulatory molecules listed in Table 3). In vitro, anti-CD3 can simulate the first signal, and anti-CD28 can simulate the second signal. For example, engineered T cells can be activated by expressed CAR. T cell activation or T cell triggering as used herein may refer to the state of T cells that have been sufficiently stimulated to induce detectable cell proliferation, cytokine production, and/or detectable effector functions.

The term “receptor” as used herein refers to a special type of protein that exists in the cell membrane or inside the cell, and can bind to a specific signal molecule outside the cell to activate a series of biochemical reactions in the cell, so that the cell produces corresponding effects on external stimuli. protein. Biologically active substances that bind to the receptor are collectively referred to as ligands. The binding of a receptor and a ligand results in a molecular conformational change, which causes cellular responses, for example mediating a process, such as intercellular signaling, intercellular adhesion, and endocytosis.

The term “co-stimulatory ligand” as used herein includes molecules on antigen-presenting cells (e.g., aAPC, dendritic cells, B cells, etc.) that specifically bind to identical co-stimulatory molecules on T cells, thereby providing signals, and mediating the T cell response together with the first signal provided by, for example, the binding of the TCR/CD3 complex and the peptide-loaded MHC molecule, including but not limited to proliferation, activation, differentiation, and the like. Co-stimulatory ligands can include but are not limited to CD7, B7-1 (CD80), B7-2 (CD86), PD-L, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin β receptor, 3/TR6, ILT3, ILT4, HVEM, agonists or antibodies binding to Toll ligand receptors and ligands that specifically binds to B7-H3. Co-stimulatory ligands also specifically include antibodies that specifically bind to co-stimulatory molecules present on T cells, such as but not limited to CD27, CD28, 4-1BB, OX40, CD30, CD40, PD-1, ICOS, lymphocyte function related Antigen-1 (LFA-1), CD2, CD7, LIGHT, NKG2C, B7-H3 and ligands that specifically bind to CD83.

The term “co-stimulatory molecule” as used herein refers to an identical binding partner on a T cell that specifically binds to a co-stimulatory ligand, thereby mediating a co-stimulatory response of the T cell, such as but not limited to proliferation. Co-stimulatory molecules include but are not limited to MHC class I molecules, BTLA and Toll ligand receptors.

“Co-stimulatory signal” as used herein refers to a signal that binds to cell stimulatory signal molecules, such as TCR/CD3, thereby leading to up- or down-regulation of T cell proliferation and/or key molecules.

The term “chimeric antigen receptor” or “CAR” as used herein refers to an engineered molecule that can be expressed by immune cells including but not limited to T cells. CAR is expressed in T cells and can redirect T cells to induce the specific killing of target cells determined by artificial receptors. The extracellular binding domain of CAR can be derived from murine, humanized or fully human monoclonal antibodies.

The term “engineered” and other grammatical forms thereof as used herein can refer to one or more changes in a nucleic acid, such as a nucleic acid within the genome of an organism. The term “engineered” can refer to changes, additions, and/or deletions of genes. Engineered cells can also refer to cells with added, deleted and/or changed genes.

As used herein, the term “cell” or “engineered cell” and other grammatical forms thereof can refer to cells of human or non-human animal origin. Engineered cells can also refer to cells expressing CAR.

The term “transfection” as used herein refers to the introduction of an exogenous nucleic acid into eukaryotic cells. Transfection can be achieved by various means known in the art, including calcium phosphate-DNA co-precipitation, DEAE-dextran-mediated transfection, polybrene-mediated transfection, electroporation, microinjection, liposome fusion, lipofection, protoplast fusion, retroviral infection and biolistics.

The term “stable transfection” or “stably transfect” refers to the introduction and integration of a exogenous nucleic acid, DNA or RNA into the genome of the transfected cell. The term “stable transfectant” refers to a cell having a foreign DNA stably integrated into its genomic DNA.

As used herein, terms “nucleic acid molecule encoding”, “encoding DNA sequence” and “encoding DNA” refer to a sequence or order of deoxyribonucleotides along a deoxyribonucleic acid chain. The order of these deoxyribonucleotides determines the order of amino acids along a polypeptide (protein) chain. Therefore, the nucleic acid sequence encodes an amino acid sequence.

The term “individual” as used herein refers to any animal, such as a mammal or a marsupial. The individual of the present invention includes, but not limited to, a human, non-human primate (such as rhesus monkey or other types of macaque), mouse, pig, horse, donkey, cattle, sheep, rat, and any kind of poultry.

The term “peripheral blood lymphocytes” (PBL) as used herein, and other grammatical forms thereof can refer to lymphocytes circulating in blood (e.g., peripheral blood). Peripheral blood lymphocytes may refer to lymphocytes that are not limited to organs. The peripheral blood lymphocytes may comprise T cells, NK cells, B cells, or any combination thereof.

The term “immune effector cell” or “immune responsive cell” as used herein may refer to cells that can trigger an immune response, including but not limited to T cells, B cells, NK cells, NKT cells, DNT cells, etc., their respective precursor cells and descendants. Immune effector cells can also refer to cells of the lymphoid or bone marrow lineage.

The term “T cell” and other grammatical forms thereof as used herein can refer to T cells of any origin. For example, the T cell may be a primary T cell, such as an autologous T cell or the like. T cells can also be of human or non-human.

As used herein, the term “T cell activation” or “T cell stimulation” and other grammatical forms thereof may refer to T cells that are sufficiently stimulated to induce detectable cell proliferation, production of cytokine, and/or detectable effector function status. In some cases, “complete T cell activation” can be similar to triggering of T cell cytotoxicity. T cell activation can be measured by using various assays known in the art. The assay may be ELISA, ELISPOT to measure cytokine secretion, flow cytometry assay (CD107) to measure intracellular cytokine expression, flow cytometry assay to measure proliferation, and cytotoxicity assay (51Cr release assay) to determine target cell elimination. In the assay, a control (non-engineered cell) is usually used to be compared with an engineered cell (CAR T) to determine the relative activation of the engineered cell relative to the control. In addition, the assay can be compared with engineered cells incubated or contacted with target cells that do not express the target antigen. For example, the comparison may be a comparison with GPC3-CART cells incubated with target cells that do not express GPC3.

When used to refer to a nucleotide sequence, the term “sequence” and other grammatical forms thereof as used herein may include DNA or RNA, and may be single-stranded or double-stranded. The nucleic acid sequence can be mutated. The nucleic acid sequence can be of any length.

The term “effective amount” as used herein refers to an amount providing therapeutic or preventive benefits.

The term “expression vector” as used herein refers to a vector containing a recombinant polynucleotide, which contains an expression control sequence operatively linked to the nucleotide sequence to be expressed. The expression vector contains sufficient cis-acting elements for expression; other elements for expression can be provided by host cells or in vitro expression systems. Expression vectors include those known in the art, such as cosmids, plasmids (e.g. naked or contained in liposomes), and viruses (e.g., lentivirus, retrovirus, adenovirus, and adeno-associated virus).

The term “lentivirus” as used herein refers to the genus of the Retroviridae family. Retroviruses are unique among retroviruses in their ability to infect non-dividing cells; they can deliver a large amount of genetic information into the DNA of host cells, thereof they are one of the most effective vehicles for gene delivery. HIV, SIV and FIV are examples of lentiviruses. Vectors derived from lentiviruses provide a means to achieve significant gene transfer in vivo.

As used herein, the term “operably linked” refers to a functional linkage between a regulatory sequence and other nucleic acid sequences, and the linkage, for example results in the expression of the latter. For example, when the first nucleic acid sequence and the second nucleic acid sequence are in a functional relationship, the first nucleic acid sequence and the second nucleic acid sequence are operably linked.

The term “promoter” as used herein is defined as a DNA sequence recognized by a synthetic mechanism of the cell or an introduced synthetic mechanism required to initiate the specific transcription of a polynucleotide sequence.

The term “vector” as used herein is a composition that contains an isolated nucleic acid and can be used to deliver the isolated nucleic acid inside a cell. Many vectors are known in the art, including but not limited to linear polynucleotides, polynucleotides related to ionic or amphiphilic compounds, plasmids, and viruses. Therefore, the term “vector” includes autonomously replicating plasmids or viruses. The term should also be interpreted to include non-plasmid and non-viral compounds facilitating the transfer of nucleic acids into cells, such as polylysine compounds, liposomes, and the like. Examples of viral vectors include, but are not limited to, adenovirus vectors, adeno-associated virus vectors, retroviral vectors, and the like.

As used herein, the term sequence “identity” determines the percent identity by comparing two best-matched sequences over a comparison window (for example, at least 20 positions), where the portion of the polynucleotide or polypeptide sequence in the comparison window may include additions or deletions (i.e. gaps), such as 20% or less gaps (e.g., 5 to 15%, or 10 to 12%) for the two sequences that best match, compared with the reference sequence (which does not contain additions or deletions). The percentage is usually calculated by determining the number of positions where the same nucleic acid base or amino acid residue occurs in the two sequences to produce the number of correct matching positions. The number of correct matching positions is divided by the total number of positions in the reference sequence (i.e., the window size), and multiply the result by 100 to produce the percentage of sequence identity.

The terms “IL12”, “interleukin-12” used herein can be used interchangeably and have the same meaning. The term “IL12” as used herein is an immunomodulatory factor with multiple biological activities. For example, the term “IL12” can refer to human IL12 as defined in SEQ ID NO: 26 or active fragments thereof, or can refer to murine IL12 as defined in SEQ ID NO: 27 or active fragments thereof, or may also be of other species.

In some embodiments, the element used to construct the receptor specifically binding to GPC3 or IL12 may be naturally occurring, for example, it may be isolated or purified from mammals; or it may also be artificially prepared, for example, recombinant elements or IL12 can be produced according to conventional genetic engineering recombination techniques. Preferably, recombinant elements or IL12 can be used in the present invention.

Based on the aforementioned elements or IL12 polypeptide sequences, amino acid sequences formed by substitution, deletion or addition of one or more amino acid residues are also included in the present invention. Appropriate replacement of amino acids is a well-known technique in the art, which can be readily implemented and ensures that biological activities of the resulting molecule will not be altered. Based on such techniques, a skilled person in the art will realize that generally, changing a single amino acid in a non-essential region of a polypeptide does not substantially change the biological activity.

Biologically active fragments of all the elements or IL12 polypeptides can be used in the present invention. The biologically active fragment mentioned herein means a polypeptide that is a part of the full-length polypeptide, while still retains all or part of the functions of the full-length polypeptide. Normally, the biologically active fragment retains at least 50% of the activity of the full-length polypeptide. Under preferred conditions, the active fragment can retain 60%, 70%, 80%, 90%, 95%, 99%, or 100% of the activity of the full-length polypeptide.

Based on the various elements or IL12 polypeptide sequences, modified or improved polypeptides can also be used in the present invention. For example, a polypeptide can be modified or improved to promote half-life, effectiveness, metabolism, and/or polypeptide effectiveness thereof. In other words, any variation that does not affect the biological activity of a polypeptide can be used in the present invention.

The term “tumor” as used herein refers to a disease characterized by the pathological proliferation of cells or tissues, and subsequent migration or invasion into other tissues or organs. Tumor growth is usually uncontrolled and progressive, and does not induce or inhibit normal cell proliferation. Tumors can affect a variety of cells, tissues or organs, including but not limited to bladder, breast, esophagus, intestine, kidney, liver, lung, lymph nodes, nerve tissue, ovary, pancreas, prostate, skeletal muscle, skin, spinal cord, spleen, stomach, uterine organs, or tissues or corresponding cells. The tumor of the present invention may include, but is not limited to, liver cancer, stomach cancer, lung cancer, breast cancer, head and neck cancer, bladder cancer, ovarian cancer, cervical cancer, kidney cancer, pancreatic cancer, cervical cancer, liposarcoma, melanoma, and adrenal carcinoma, schwannoma, malignant fibrous histiocytoma, esophageal cancer. Preferably, the “tumor” includes, but is not limited to: liver cancer, gastric cancer, lung cancer, and breast cancer.

The term “enhancement of immune effector cell function” as used herein includes, for example, enhancement of T cell function. Taking T cells as an example, enhancing T cell functions includes inducing, causing or stimulating T cells to have sustained or enhanced biological functions, or renewing or reactivating exhausted or inactive T cells. Examples of enhancing T cell function include: compared with the pre-intervention level, increased interferon secretion by CD8+ T cells, increased proliferation, and increased antigen reactivity (e.g., virus or pathogen clearance rate). In one embodiment, the enhancement is at least 50%, or 60%, 70%, 80%, 90%, 100%, 120%, 150%, 200%. The way to measure such enhancement is known to a skill person in the art.

The term “exogenous” as used herein refers to a nucleic acid molecule or polypeptide not endogenously expressed in a cell, or the expression level of which is insufficient to achieve the function that it exhibits when it is overexpressed. Thus, “exogenous” includes recombinant nucleic acid molecules or polypeptides expressed in cells, such as exogenous, heterologous and overexpressed nucleic acid molecules and polypeptides.

The term “receptor” as used herein refers to a polypeptide, or part thereof, selectively binding to one or more ligands on the cell membrane.

In some embodiments, the antigen-binding receptor of the present invention is a chimeric antigen receptor. The term “Chimeric Antigen Receptor (CAR)” as used herein refers to a tumor antigen binding domain fused to an intracellular signaling domain that can activate T cells. The extracellular binding domain of CAR is generally derived from mouse or humanized or human monoclonal antibodies.

Chimeric antigen receptors usually contain extracellular antigen binding regions. In some embodiments, the extracellular antigen binding region may be of full human. In other cases, the extracellular antigen binding region can be humanized. In other cases, the extracellular antigen binding region may be of murine origin, or the chimera in the extracellular antigen binding region consists of amino acid sequences from at least two different animals. In some embodiments, the extracellular antigen binding region may be of non-human.

A variety of antigen binding regions can be designed. Non-limiting examples include single chain variable fragments (scFv) derived from antibodies, antigen binding regions (Fab) selected from libraries, single domain fragments, or natural ligands that engage cognate receptors thereof. In some embodiments, the extracellular antigen binding region may comprise scFv, Fab, or natural ligands, and any derivatives thereof. The extracellular antigen binding region may refer to a molecule other than the intact antibody, which may comprise a part of the intact antibody and can bind to the antigen to which the intact antibody binds. Examples of antibody fragments may include, but are not limited to, Fv, Fab, Fab′, Fab′-SH, F(ab′)2; bifunctional antibodies, linear antibodies; single-chain antibody molecules (such as scFv); and multispecific antibodies formed from antibody fragments.

Extracellular antigen binding regions, such as scFv, Fab, or natural ligands, can be part of a CAR that determines antigen specificity. The extracellular antigen binding region can bind to any complementary target. The extracellular antigen binding region can be derived from antibodies with known variable region sequences. The extracellular antigen binding region can be obtained from available antibody sequences from mouse hybridomas. Alternatively, the extracellular antigen binding region can be obtained from total extracellular cleavage sequencing of tumor cells or primary cells, such as tumor infiltrating lymphocytes (TIL).

In some cases, the binding specificity of the extracellular antigen binding region can be determined by complementarity determining regions or CDRs, such as light chain CDRs or heavy chain CDRs. In many cases, the binding specificity can be determined by the light chain CDR and the heavy chain CDR. Compared with other reference antigens, the combination of a given heavy chain CDRs and light chain CDRs can provide a given binding pocket, which can confer greater affinity and/or specificity to the antigen (e.g., GPC3). For example, CDRs specific to Glypican-3 can be expressed in the extracellular binding region of the CAR, so that the GPC3-targeted CAR can target immune effector cells to tumor cells expressing GPC3.

In certain aspects of any embodiments disclosed herein, the extracellular antigen binding region, such as a scFv, may comprise a light chain CDR specific for the antigen. The light chain CDR may be the complementarity determining region of the scFv light chain of an antigen binding unit, such as a CAR. The light chain CDR may comprise a sequence of consecutive amino acid residues, or a sequence of two or more consecutive amino acid residues separated by a non-complementarity determining region (e.g., a framework region). In some cases, light chain CDR may include two or more light chain CDRs, which may be referred to as light chain CDR-1, CDR-2, and the like. In some cases, the light chain CDR may comprise three light chain CDRs, which may be referred to as light chain CDR-1, light chain CDR-2, and light chain CDR-3, respectively. In some examples, a group of CDRs present on a common light chain can be collectively referred to as light chain CDRs.

In certain aspects of any embodiments disclosed herein, the extracellular antigen binding region, such as a scFv, may comprise a heavy chain CDR specific for an antigen. The heavy chain CDR may be the heavy chain complementarity determining region of an antigen binding unit, such as a scFv. The heavy chain CDR may comprise a continuous sequence of amino acid residues, or a continuous sequence of two or more amino acid residues separated by a non-complementarity determining region (such as a framework region). In some cases, a heavy chain CDR may comprise two or more heavy chain CDRs, which may be referred to as heavy chain CDR-1, CDR-2, and the like. In some cases, the heavy chain CDR may include three heavy chain CDRs, which may be referred to as heavy chain CDR-1, heavy chain CDR-2, and heavy chain CDR-3, respectively. In some cases, a group of CDRs present on a common heavy chain can be collectively referred to as a heavy chain CDR.

By using genetic engineering, the extracellular antigen binding region can be modified in various ways. In some cases, the extracellular antigen binding region can be mutated, so that the extracellular antigen binding region can be selected to have a higher affinity for its target. In some cases, the affinity of the extracellular antigen binding region for its target can be optimized for targets that can be expressed at low levels on normal tissues. Such optimization can be performed to minimize potential toxicity. In other cases, clones of extracellular antigen-binding regions with higher affinity for the membrane-bound form of the target may be superior to their soluble form counterparts. Such modification can be performed since different levels of targets in a soluble form can still be detected, and targeting thereof can cause undesirable toxicity.

In some cases, the extracellular antigen binding region includes a hinge or spacer. The terms hinge and spacer can be used interchangeably. The hinge can be considered as part of the CAR used to render flexibility to the extracellular antigen binding region. In some cases, hinges can be used to detect CARs on the cell surface, especially when antibodies that detect extracellular antigen binding regions are ineffective or unavailable. For example, it may be necessary to optimize the length of the hinge derived from immunoglobulin, depending on the location of the epitope on the target targeted by the extracellular antigen binding region.

In some cases, the hinge may not belong to immunoglobulin, but belong to another molecule, such as the natural hinge of CD8a molecule. The CD8a hinge may contain cysteine and proline residues that are known to play a role in the interaction of CD8 co-receptors and MHC molecules. The cysteine and proline residues can affect the performance of the CAR.

The CAR hinge can be adjustable in size. The morphology of the immune synapse between the immune effector cell and the target cell also defines the distance that cannot be functionally bridged by CAR due to the distal membrane epitopes of the target molecule on the cell surface, therefore, the distance between synapses cannot reach the approximate value of signal transmission even with the short hinge CAR. Similarly, for the CAR targeting epitope at the proximal end of the membrane, signal output can be observed only in the context of the long hinge CAR. The hinge can be adjusted according to the used extracellular antigen binding region. The hinge can be of any length.

The transmembrane domain can anchor the CAR to the plasma membrane of the cell. The natural transmembrane portion of CD28 can be used in a CAR. In other cases, the natural transmembrane portion of CD8a can also be used in a CAR. “CD8” can be a protein that has at least 85, 90, 95, 96, 97, 98, 99, or 100% identity with NCBI reference number: NP_001759 or a fragment having stimulating activity. “CD8 nucleic acid molecule” can be a polynucleotide encoding a CD8 polypeptide. In some cases, the transmembrane region can be the natural transmembrane portion of CD28. “CD28” can refer to a protein having at least 85, 90, 95, 96, 97, 98, 99, or 100% identity with the reference number of NCBI: NP_006130 or a fragment thereof with stimulating activity. A “CD28 nucleic acid molecule” may be a polynucleotide encoding a CD28 polypeptide. In some cases, the transmembrane portion may comprise a CD8a region.

The intracellular signal domain of the CAR may be responsible for activating at least one of the effector functions of the immune effector cells in which the CAR has been placed. CAR can induce effector functions of T cells, for example, the effector function is cytolytic activity or auxiliary activity, including secretion of cytokines. Therefore, the term “intracellular signal domain” refers to the part of a protein that transduces effector function signals and guides cells to perform specific functions. Although the entire intracellular signaling region can usually be used, in many cases it is not necessary to use the entire chain of signaling domains. In some cases, truncated portions of intracellular signaling regions are used. In some cases, the term intracellular signaling domain is therefore intended to include any truncated portion of the intracellular signaling region which is sufficient to transduce effector function signals.

Preferred examples of signal domains used in CAR may include cytoplasmic sequences of T cell receptor (TCR) and co-receptors synergistically acting to initiate signal transduction after target-receptor binding, as well as any derivatives or variant sequence thereof and any synthetic sequence with the same functionality of these sequences.

In some cases, the intracellular signaling domain may contain a known immunoreceptor tyrosine activation motif (ITAM) signaling motif. Examples of ITAMs containing cytoplasmic signaling sequences include functional signaling domains derived from proteins of TCRζ, FcRγ, FcRβ, CD3γ, CD3δ, CD3ε, CD5, CD22, CD79a, CD79b, CD66d DAP10, or DAP12. However, in a preferred embodiment, the intracellular signaling domain is derived from CD3ζ chain.

An example of a T cell signaling domain containing one or more ITAM motifs is CD3ζ domain, also known as T cell receptor T3ζ chain or CD247. This domain is a part of the T cell receptor-CD3 complex, and plays an important role in combining the antigen recognition of several intracellular signal transduction pathways with the main effect activation of T cells. As used herein, CD3ζ mainly refers to human CD3ζ and isoforms thereof, as known from Swissprot entry P20963, including proteins with substantially the same sequence. As a part of the chimeric antigen receptor, it shall be noticed once again that the whole T cell receptor T3ζ chain is not required, and any derivative containing the signaling domain of the T cell receptor T3ζ chain may be suitable, including any functional equivalents thereof.

The intracellular signaling domain can be selected from any one of the domains in Table 1. In some cases, the domain can be modified so that the identity with the reference domain can be about 50% to about 100%. Any one of the domains in Table 1 can be modified so that the modified form can comprise about 50, 60, 70, 80, 90, 95, 96, 97, 98, 99 or up to about 100% identity.

The intracellular signaling region of the CAR may further include one or more co-stimulatory domains. The intracellular signaling region may contain a single co-stimulatory domain, such as the ζ chain (first-generation of CAR) or in combination with CD28 or 4-1BB (second-generation of CAR). In other examples, the intracellular signaling region may contain two co-stimulatory domains, such as CD28/OX40 or CD28/4-1BB (third generation).

Together with intracellular signaling domains such as CD8, these co-stimulatory domains can produce downstream activation of the kinase pathway, thereby supporting gene transcription and functional cellular responses. The co-stimulatory domain of a CAR can activate CD28 (phosphatidylinositol-4,5-bisphosphate 3-kinase) or 4-1BB/OX40 (TNF-receptor-related factor adaptor protein) pathways, as well as proximal signaling proteins related to MAPK and Akt activation.

In some cases, the signal generated by the CAR may be combined with auxiliary or co-stimulatory signals. For co-stimulatory signal domains, the chimeric antigen receptor-like complex can be designed to contain several possible co-stimulatory signal domains. As is well known in the art, in naive T cells, T cell receptor engagement alone is not sufficient to induce the complete activation of T cells into cytotoxic T cells. The activation of intact producer T cells requires a second co-stimulatory signal. Several receptors that provide co-stimulation to T cell activation have been reported, including but not limited to CD28, OX40, CD27, CD2, CD5, ICAM-1, LFA-1 (CD11a/CD18), 4-1BBL, MyD88 and 4-1BB. All of the signaling pathways used by these co-stimulatory molecules can synergistically act with the master T cell receptor activation signal. The signals provided by these co-stimulatory signaling regions can synergistically act with main effect activation signals derived from one or more ITAM motifs (such as the CD3zeta signaling domain), and can complete the requirement of T cell activation.

In some cases, the addition of co-stimulatory domains to chimeric antigen receptor-like complexes can enhance the efficacy and durability of engineered cells. In another embodiment, the T cell signaling domain and the co-stimulatory domain are fused to each other to form a signaling region.

TABLE 1
Co-stimulatory domain

| Gene Marker | Abbreviation | Name |
|---|---|---|
| CD27 | CD27; T14; S152; Tp55; | CD27 molecule |
| | TNFRSF7; S152. LPFS2 | |
| CD28 | Tp44; CD28; CD28 antigen | CD28 molecule |
| TNFRSF9 | ILA; 4-1BB; CD137; CDw137 | Tumor Necrosis Factor Receptor |
| | | Superfamily Member 9 |
| TNFRSF4 | OX40; ACT35; CD134; IMD16; | Tumor Necrosis Factor Receptor |
| | TXGP1L | Superfamily Member 4 |
| TNFRSF8 | CD30; Ki-1; D1S166E | Tumor Necrosis Factor Receptor |
| | | Superfamily Member 8 |
| CD40LG | IGM; IMD3; TRAP; gp39; | CD40 ligand |
| | CD154; CD40L; HIGM1; | |
| | T-BAM; TNFSF5; hCD40L | |
| ICOS | AILIM; CD278; CVID1 | Inducible T cell costimulator |
| ITGB2 | LAD; CD18; MF17; MFI7; | Integrin β2 (complement |
| | LCAMB; LFA-1; MAC-1 | component 3 receptor 3 and 4 |
| | | subunits) |
| CD2 | T11; SRBC; LFA-2 | CD2 molecule |
| CD7 | GP40; TP41; Tp40; LEU-9 | CD7 molecule |
| KLRC2 | NKG2C; CD159c; NKG2-C | Killer cell lectin-like receptor |
| | | subfamily C, Member 2 |
| TNFRSF18 | AITR; GITR; CD357; GITR-D | Tumor Necrosis Factor Receptor |
| | | Superfamily Member 18 |
| TNFRSF14 | TR2; ATAR; HVEA; HVEM; | Tumor Necrosis Factor Receptor |
| | CD270; LIGHTR | Superfamily Member 14 |
| HAVCR1 | TIM; KIM1; TIM1; CD365; | Hepatitis A Virus Cell Receptor 1 |
| | HAVCR; KIM-1; TIM-1; TIMD1; | |
| | TIMD-1; HAVCR-1 | |
| LGALS9 | HUAT; LGALS9A, Galectin-9 | Lectin, galactoside binding, |
| | | soluble, 9 |
| CD83 | BL11; HB15 | CD83 molecule |

The term “modulation” as used herein refers to a positive or negative change. Examples of adjustments include changes of 1%, 2%, 10%, 25%, 50%, 75%, or 100%.

The term “treatment” as used herein refers to clinical intervention in the process of trying to change a person or treating a disease caused by cells, which can be used for prevention or intervention in the clinical pathological process. The therapeutic effects include, but not limited to, preventing the occurrence or recurrence of the disease, reducing the symptoms, reducing the direct or indirect pathological consequences of any disease, preventing metastasis, slowing down the progression of the disease, improving or relieving the condition, relieving or improving the prognosis, etc.

The term “immune-compromised” as used herein means that a subject has immunodeficiency and is easily infected. Organisms that cause opportunistic infections usually do not cause illness with a healthy immune system, but can infect people with a weakened or suppressed immune system.

The term “constitutive expression” as used herein refers to the expression under all physiological conditions.

The term “induced expression” as used herein refers to the expression under certain conditions, such as when T cells bind to an antigen. A person skilled in the art will know how to perform a conventional “induction of expression”.

Immune Effector Cells

In some embodiments, the present invention provides an immune effector cell expressing exogenous IL12 and a receptor that specifically binds to GPC3.

In some embodiments, the exogenous IL12 is constitutively expressed or inducibly expressed; preferably inducibly expressed. For example, an inducible promoter; preferably NFAT6 promoter is used.

In some embodiments, the transmembrane region of the antigen-binding receptor may be selected from the transmembrane region of a protein such as CD8 or CD28. The human CD8 protein is a heterodimer, composed of two chains, αβ or γδ. In some embodiments, the transmembrane region is selected from the transmembrane region of CD8α or CD28. In addition, the CD8α hinge region (hinge) is a flexible region. Therefore, CD8 or CD28 and the transmembrane region as well as the hinge region are used to connect target recognition domain scFv of the antigen-binding receptor CAR and the intracellular signal region.

The intracellular signal domain of the present invention can be selected from CD3ζ, FcεRIγ, CD28 co-stimulatory signal domain, CD137 co-stimulatory signal domain, and combinations thereof. The CD3 molecule is composed of five subunits, in which the CD3ζ subunit (also known as CD3 zeta, abbreviated as Z) contains three ITAM motifs, which are important signaling region in the TCR-CD3 complex. In addition, as mentioned earlier, CD28 and CD137 are co-stimulatory signal molecules, and after binding to respective ligands, the co-stimulation effect produced by their intracellular signal segment can cause the continuous proliferation of immune effector cells (mainly T lymphocytes), increase the level of cytokines such as IL-2 and IFN-γ secreted by immune effector cells, and improve the survival and anti-tumor effects of CAR immune effector cells in the body. In some embodiments, the intracellular signaling domain is a CD3ζ signal domain or a combination of a CD3ζ signal domain and other co-stimulatory signals, such as CD28.

In some embodiments, the immune effector cell of the present invention may include an expression construct, in which there are elements connected sequentially in the following manner: antibody, CD28 co-stimulatory signal domain, CD3ζ, and NFAT6, IL12 expression unit reversely linked with the foregoing elements. Preferably, the antibody and the CD28 co-stimulatory signal domain are connected through the CD8α transmembrane region and the CD8α hinge region.

In some embodiments, the nuclear factor of activated T cells (NFAT) plays an important role in the transcriptional expression of cytokines during the activation of T cell. Therefore, the present inventors placed the IL12 encoding sequence under the control of the NFAT6 promoter, so that only when CAR-T cells contact an antigen and trigger T cell activation, IL12 can be expressed at a high level.

The NFAT6 promoter is a promoter composed of 6 NFAT binding sites and the minimal promoter of IL2 linked in tandem (Hooijberg E, Bakker A Q, Ruizendaal J J, Spits H. NFAT-controlled expression of GFP permits visualization and Isolation of antigen-stimulated primary human Tcells. Blood. 2000 Jul. 15; 96(2):459-66), which can be used to regulate the expression of cytokines, such as IL12 in T lymphocytes, such as TCR-T (Zhang L, Kerkar S P, Yu Z, Zheng Z, Yang S, RestifoN P, Rosenberg S A, Morgan R A. Improving adoptive T cell therapy by targeting and controlling IL-12 expression to the tumor environment. Mol Ther. 2011 April; 19(4):751-9).

According to one aspect of the present invention, the present invention also includes a nucleic acid encoding the antigen-binding receptor. The present invention also relates to variants of the above-mentioned polynucleotides, which encode polypeptides having the same amino acid sequence as the present invention, or polypeptide fragments, analogs and derivatives.

The present invention also provides a vector containing the nucleic acid encoding the antigen-binding receptor protein expressed on the surface of immune effector cells. In a specific embodiment, the vector used in the present invention is a lentiviral plasmid vector pRRLSIN-cPPT.PGK-GFP.WPRE. It should be understood that other types of viral vectors and non-viral vectors are also applicable.

The present invention also includes a virus containing the above-mentioned vectors. The virus of the present invention includes a packaged virus with infectivity, and also includes a virus to be packaged that contains necessary components for packaging as a virus with infectivity. Other viruses known in the art that can be used to transfer a foreign gene into immune effector cells and corresponding plasmid vectors can also be used in the present invention.

The immune effector cell of the present invention is transduced with a construct capable of expressing an antigen-binding receptor and exogenous IL12, or an expression vector, or a virus containing the plasmid. Conventional nucleic acid transduction methods in the art, including non-viral and viral transduction methods, can be used in the present invention.

The immune effector cell of the present invention can also express another antigen-binding receptor besides the above-mentioned antigen-binding receptor.

The immune effector cell of the present invention can also express a safety switch; and preferably, the safety switch includes: iCaspase-9, Truncated EGFR or RQR8.

Nucleic Acid

A transgene encoding a target binding antigen receptor or CAR can be incorporated into the cell. For example, the transgene can be incorporated into immune effector cells, such as T cells. When inserted into a cell, the transgene can be a complementary DNA (cDNA) fragment, which is a copy of messenger RNA (mRNA); or the gene itself (with or without introns) located in the original region of its genomic DNA.

Nucleic acids encoding transgenic sequences, such as DNA, can be randomly inserted into the chromosomes of cells. The random integration can be achieved by any method of introducing nucleic acid (e.g., DNA) into cells. For example, the method may include, but not limited to, electroporation, ultrasound, using gene guns, lipofection, calcium phosphate transfection, using dendrimers, microinjection, and using virus vectors including adenovirus, AAV and retroviral vectors, and/or type II ribozyme.

The DNA encoding the transgene can also be designed to include a reporter gene, so that the presence of the transgene or its expression product can be detected by the activation of the reporter gene. Any reporter gene can be used, such as those described above. By selecting cells in the cell culture in which the reporter gene has been activated, cells containing the transgene can be selected.

The expression of CAR can be verified by expression assays, such as qPCR or by measuring the level of RNA. The expression level can also indicate copy number. For example, if the expression level is very high, this can indicate that more than one copy of the CAR is integrated into the genome. Alternatively, the high expression can indicate that the transgene is integrated in a highly transcribed region, such as a region near a highly expressed promoter. The expression can also be verified by measuring protein levels, for example by Western blotting.

In some embodiments, the immune effector cells of the present invention may contain one or more transgenes. The one or more transgenes can express the CAR protein, and the CAR protein recognizes and binds to at least one epitope on the antigen or binds to a mutant epitope on the antigen. The CAR can be a functional CAR. In some embodiments, the immune effector cell of the present invention may comprise one or more CARs, or it may comprise a single CAR and a secondary engineered receptor.

As mentioned above, the transgene can be inserted into the genome of immune responsive cells in a random or site-specific manner. For example, the transgene can be inserted into a random site in the genome of an immune cell. These transgenes can be functional, for example, fully functional when inserted any site in the genome. For example, the transgene can encode its own promoter, or can be inserted into a position controlled by its internal promoter. Alternatively, the transgene can be inserted into a gene, such as an intron of a gene or an exon, a promoter, or a non-coding region of a gene. Transgenes can be inserted, so that genes, such as endogenous immune checkpoints, can be disrupted by the insertion.

In some cases, more than one copy of the transgene can be inserted into multiple random sites within the genome. For example, multiple copies can be inserted at random locations in the genome. This may result in an increase in overall expression compared with one random insertion of the transgene. Alternatively, a copy of the transgene can be inserted into the gene, and another copy of the transgene can be inserted into a different gene. The transgene can be targeted, so that it can be inserted into a specific site in the genome of immune responsive cells.

In some cases, a polynucleic acid containing a sequence encoding a receptor binding to an antigen may take the form of a plasmid vector. The plasmid vector may contain a promoter. In some cases, the promoter can be constitutive. In some cases, the promoter is inducible. The promoter can be or can be derived from CMV, U6, MND or EF1a. In some cases, the promoter may be adjacent to the CAR sequence. In some cases, the plasmid vector also contains a splice acceptor. In some cases, the splice acceptor may be adjacent to the CAR sequence. The promoter sequence can be a PKG or MND promoter. The MND promoter may be a synthetic promoter containing the U3 region of MoMuLV LTR modified with an enhancer of myeloproliferative sarcoma virus.

In some cases, polynucleotide encoding target receptors can be designed for delivery to cells by non-viral techniques. In some cases, the polynucleotide can be a compatible reagent of good manufacturing practice (GMP).

The expression of the polynucleotide encoding the target binding antigen receptor or CAR can be controlled by one or more promoters. Promoters can be ubiquitous, constitutive (unrestricted promoters that allow continuous transcription of related genes), tissue-specific promoters or inducible promoters. The expression of transgenes inserted adjacent or close to the promoter can be regulated. For example, the transgene can be inserted near or beside a ubiquitous promoter. Some ubiquitous promoters can be CAGGS promoter, hCMV promoter, PGK promoter, SV40 promoter or ROSA26 promoter.

The promoter can be endogenous or exogenous. For example, one or more transgenes can be inserted in the vicinity or proximity of the endogenous or exogenous ROSA26 promoter. In addition, the promoter may be specific for immune responsive cells. For example, one or more transgenes can be inserted in the vicinity or proximity of the porcine ROSA26 promoter.

Tissue-specific promoters or cell-specific promoters can be used to control the location of expression. For example, one or more transgenes can be inserted in the vicinity or proximity of a tissue-specific promoter. The tissue-specific promoter can be FABP promoter, Lck promoter, CamKII promoter, CD19 promoter, keratin promoter, albumin promoter, aP2 promoter, insulin promoter, MCK promoter, MyHC promoter, WAP Promoter, or Col2A promoter.

Inducible promoters can also be used. If necessary, these inducible promoters can be turned on and off by adding or removing inducers. It is expected that the inducible promoter may be, but not limited to, Lac, tac, trc, trp, araBAD, phoA, recA, proU, cst-1, tetA, cadA, nar, PL, cspA, T7, VHB, Mx and/or Trex.

Immune Cell Inducible Promoter

As used herein, the term “inducible promoter” is a controlled promoter that does not express or under-expresses the gene operably linked to it before the expected condition is achieved, and when the expected condition is achieved, expresses or expresses at a high level the gene to which it is operably linked. For example, in some embodiments, the inducible promoter of the present application does not express or under-expresses the gene operably linked to it under normal or high oxygen content conditions in the cell, while expresses or expresses at a high level the gene to which it is operably linked under hypoxic conditions in the response to the decreased oxygen content in the cell. In some embodiments, the inducible promoter used herein includes hypoxia-inducible transcription factor-1α (HIF-1α). In some embodiments, the term “inducible promoter” as used herein refers to an “immune cell inducible promoter”, which does not express or expresses at low level the gene to which it is operably linked before the immune effector cell contacts the antigen or when the immune effector cell is not activated, while only drives high-level expression of the gene to which it is operably linked only when the immune effector cells contact antigen or immune effector cells are activated or under conditions such as hypoxia. In some embodiments, the “immune cell inducible promoter” includes a NFAT (nuclear factor of activated T cell)-type promoter.

As used herein, “NFAT-type promoter” refers to a type of promoter that regulates the expression of a gene to which it is operably linked based on the NFAT binding activity.

NFAT is a collective name for a family of transcription factors that play an important role in the immune response. One or more members of the NFAT family are expressed in most cells of the immune system. NFAT is also involved in the development of the heart, skeletal muscle and nervous system.

The NFAT transcription factor family consists of five members, NFATc1, NFATc2, NFATc3, NFATc4 and NFATS. NFATc1 to NFATc4 are regulated by calcium signals. The Calcium signal is essential for NFAT activation, since calmodulin (CaM) activates serine/threonine phosphatase calcineurin (CN). The activated CN rapidly dephosphorylates the serine-rich region (SRR) and SP repeats at the amino terminus of the NFAT protein, leading to conformational changes, exposing nuclear localization signals, and leading to the import of NFAT into the nucleus.

Based on the role of NFAT in the transcriptional expression of cytokines during the activation of T cells, it can be used to regulate the immune cell inducible promoter described herein, so that when immune effector cells are exposed to antigen and activated, the gene to which it is operably linked can be expressed or high-level expressed. In some embodiments, the “NFAT-type promoter” described herein may include more than one NFAT binding site. For example, the “NFAT-type promoter” may include 2, 3, 4, 5, 6, 7, 8, 9, 10 or more NFAT binding sites. In some embodiments, the “NFAT-type promoter” may be a promoter composed of a plurality of NFAT binding sites and a promoter, such as the IL2 minimal promoter linked in tandem. In some embodiments, the NFAT-type promoter described herein includes 6 NFAT binding sites, designated as (NFAT)6. For convenience, the (NFAT)6 is also referred to as NFAT6. In some embodiments, the NFAT6 also represents 6 repeated NFAT binding sites in the NFAT-type promoter.

In addition, the transgene sequence may also include transcription or translation regulatory sequences, such as promoters, enhancers, insulators, internal ribosome entry sites, sequences encoding 2A peptides and/or polyadenylation signals, although not required for expression.

In some cases, retroviral vectors (γ-retrovirus or lentiviral vectors) can be used to introduce transgenes into immune responsive cells. For example, the transgenes encoding a CAR or any antigen-binding receptors or variants or fragments thereof can be cloned into retroviral vectors, and can be derived by the endogenous promoters, retroviral long terminal repeats, or promotes specific to the type of the target cell. Non-viral vectors can also be used. The non-viral vector delivery system may include DNA plasmids, naked nucleic acids, and nucleic acids complexed with delivery vehicles, such as liposomes or poloxamers.

Many virus-based systems have been developed for transferring genes into mammalian cells. For example, retroviruses provide a convenient platform for gene delivery systems. The selected gene can be inserted into a vector and packaged in a retroviral particle using techniques known in the art. Vectors derived from retroviruses, such as lentiviruses are suitable tools to achieve long-term gene transfer, since the long-term and stable integration of the transgene and the propagation thereof in daughter cells thereof can be allowed. Lentiviral vectors have additional advantages over vectors derived from retroviruses, such as murine leukemia virus, since they can transduce non-proliferating cells. They also have added advantages of low immunogenicity. The advantage of adenoviral vectors is that they do not fuse into the genome of target cells, thereby bypassing negative integration-related events.

Cells can be transfected with a transgene encoding the receptor that binds to the antigen. The concentration of the transgene can range from about 100 picograms to about 50 micrograms. In some cases, the amount of nucleic acid (e.g., ssDNA, dsDNA, or RNA) introduced into the cell can be changed to optimize transfection efficiency and/or cell viability. For example, 1 microgram of dsDNA can be added to each cell sample for electroporation. In some cases, the amount of nucleic acid (e.g., double-stranded DNA) required for optimal transfection efficiency and/or cell viability varies according to cell type. In some cases, the amount of nucleic acid (e.g., dsDNA) used for each sample can directly correspond to transfection efficiency and/or cell viability. For example, a series of transfection concentrations. The transgene encoded by the vector can be integrated into the genome of the cell. In some cases, the transgene encoded by the vector is forward integrated. In other cases, the transgene encoded by the vector is reversely integrated.

In some cases, the immune responsive cells may be stem memory TSCM cells composed of CD45RO(−), CCR7(+), CD45RA(+), CD62L+(L-selectin), CD27+, CD28+ and/or IL-7Rα+, and the stem memory cells can also express CD95, IL-2Rβ, CXCR3 and/or LFA-1, and show many functional properties different from the stem memory cells. Alternatively, the immune responsive cell may also be a central memory TCM cell containing L-selectin and CCR7, where the central memory cell can secrete, for example, IL-2, but not IFNγ or IL-4. The immune responsive cells can also be effector memory TEM cells containing L-selectin or CCR7, and produce, for example, effector cytokines, such as IFNγ and IL-4.

The vector is usually delivered into an individual by systemic administration (e.g., intravenous, intraperitoneal, intramuscular, subcutaneous, or intracranial infusion) or topical application, as described below. Alternatively, the vector can be delivered to cells ex vivo. For example, cells are removed from an individual patient (e.g., lymphocytes, T cells, bone marrow aspirate, tissue biopsy), and then the cells are usually selected, have the vector incorporated and implanted into the patient's body. The cells can be expanded before or after the selection.

Suitable immune responsive cells for expressing receptors that bind to an antigen may be cells that are autologous or non-autologous to the individual in need thereof.

A suitable source of immune effector cells can be obtained from an individual. In some cases, T cells can be obtained. The T cells can be obtained from many sources, including PBMC, bone marrow, lymph node tissue, umbilical cord blood, thymus tissue, and tissue from infection sites, ascites, pleural effusion, spleen tissue, and tumors. In some cases, any techniques known to a skilled person in the art, such as Ficoll™ isolation, can be used to obtain T cells from blood collected from the individual. In one embodiment, cells from the circulating blood of the individual are obtained by Apheresis. Apheresis products usually contain lymphocytes, including T cells, monocytes, granulocytes, B cells, other nucleated white blood cells, red blood cells and platelets. In one embodiment, the cells collected by apheresis can be washed to remove the plasma fraction and placed in a suitable buffer or medium for subsequent processing steps.

Alternatively, cells can be derived from healthy donors, from patients diagnosed with cancer, or patients diagnosed with infection. In some embodiments, the cell may be a part of a mixed cell population with different phenotypic characteristics. Cell lines can also be obtained from transformed T cells according to the aforementioned method. Cells can also be obtained from cell therapy banks. Modified cells resistant to immunosuppressive therapy can be obtained by any of the methods described herein. It is also possible to select a suitable cell population before modification. Engineered cell populations can also be selected after modification. Engineered cells can be used for autologous transplantation. Alternatively, the cells can be used for allogeneic transplantation. In some cases, the cells are administered to a patient, whose sample is used to identify cancer-related target sequences. In other cases, the cells are administered to a patient other than those whose samples are used to identify cancer-related target sequences.

In some cases, suitable primary cells include peripheral blood mononuclear cells (PBMC), peripheral blood lymphocytes (PBL) and other blood cell subpopulations, such as but not limited to T cells, natural killer cells, monocytes, natural Killer T cells, monocyte precursor cells, hematopoietic stem cells or non-pluripotent stem cells. In some cases, the cells can be any immune cells, including any T cells, such as tumor infiltrating cells (TIL), such as CD3+ T cells, CD4+ T cells, CD8+ T cells, or T cells of any other type. T cells may also include memory T cells, memory stem T cells, or effector T cells. It is also possible to select T cells from a large population, for example from whole blood. T cells can also be expanded from large populations. T cells may also be of a specific population and phenotype. For example, T cells can be of a phenotype including CD45RO(−), CCR7(+), CD45RA(+), CD62L(+), CD27(+), CD28(+), and/or IL-7Rα(+). Suitable cells can comprise one or more markers selected from the following list: CD45RO(−), CCR7(+), CD45RA(+), CD62L(+), CD27(+), CD28(+) and/or IL-7Rα(+). Suitable cells also include stem cells, such as, embryonic stem cells, induced pluripotent stem cells, hematopoietic stem cells, neuronal stem cells, and mesenchymal stem cells. Suitable cells may include any number of primary cells, such as human cells, non-human cells, and/or mouse cells. Suitable cells may be progenitor cells. Suitable cells can be derived from a subject (e.g., patient) to be treated.

The amount of therapeutically effective cells required in a patient can vary depending on the viability of the cells and the efficiency with which the cells are genetically modified (for example, the efficiency with which the transgene is integrated into one or more cells, or the expression level of the protein encoded by the transgene). In some cases, the product (e.g., multiplication) of cell viability after genetic modification and the integration efficiency of transgene may correspond to the therapeutic amount of cells available for administration to a subject. In some cases, the increase in cell viability after genetic modification may correspond to a decrease in the necessary amount of cells that is effective for the patient when the treatment is administered. In some cases, an increase in the efficiency of integration of the transgene into one or more cells may correspond to a decrease in the necessary therapeutically effective number of cells administered in a patient. In some cases, the determination of the necessary therapeutically effective amount of cells can include the determination of functions related to changes in the cells over time. In some cases, the determination of the necessary therapeutically effective amount of cells may include the determination of functions corresponding to changes in the efficiency of integrating the transgene into one or more cells based on time-related variables (e.g., culture time for cells, electroporation time, stimulation time for cells). In some cases, a therapeutically effective cell may be a cell population that contains about 30% to about 100% expression of antigen-binding receptors on the cell surface. In some cases, as measured by flow cytometry, therapeutically effective cells can express the antigen-binding receptor on the cell surface by about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9% or more than about 99.9%.

In some cases, when the antigen-binding receptor is present on the plasma membrane of a cell, and when it is activated by binding to a target, the toxicity to target cells with the target expressed in the cell surface will be caused. For example, in some cases, the cell may be a cytotoxic cell (e.g., NK cell or cytotoxic T lymphocyte). When the antigen-binding receptor as described herein is activated by binding to its target, it can increase the cytotoxic activity of cytotoxic cells to target cells. For example, in some cases, when the antigen-binding receptor described herein is activated by binding to its target, it can increase cytotoxicity by at least 10%, at least 15%, at least 20%, at least 25%, at least 30%, at least 40%, at least 50%, at least 75%, at least 2 times, at least 2.5 times, at least 5 times, at least 10 times or more 10 times, compared with the cytotoxicity to cells without the target.

Pharmaceutical Composition

The immune effector cells of the present invention can be used to prepare a pharmaceutical composition. In addition to including an effective amount of immune effector cells, the pharmaceutical composition may also include a pharmaceutically acceptable carrier. The term “pharmaceutically acceptable” means that when the molecular entity and composition are properly administered to animals or humans, they will not produce adverse, allergic or other undesired reactions.

Specific examples of some substances that can be used as pharmaceutically acceptable carriers or components thereof are antioxidants; preservatives; pyrogen-free water; isotonic salt solutions; and phosphate buffers and the like.

The composition of the present invention can be prepared into various dosage forms according to needs, and a doctor can determine the beneficial dosage for a patient according to factors, such as the type of the patient, age, weight, general disease condition, and administration method. The mode of administration can be, for example, parenteral administration (such as injection) or other treatment modes.

“Parenteral” administration of an immunogenic composition includes, for example, subcutaneous (s.c.), intravenous (i.v.), intramuscular (i.m.) or intrasternal injection or infusion techniques.

The formulation containing a population of immune responsive cells administered to an individual contains a plurality of immune responsive cells effective in treating and/or preventing a specific indication or disease. Therefore, a therapeutically effective population of immune responsive cells can be administered to an individual. Generally, a preparation containing about 1×10⁴ to about 1×10¹⁰ immune responsive cells is administered. In most cases, the formulation will contain about 1×10⁵ to about 1×10⁹ immune responsive cells, about 5×10⁵ to about 5×10⁸ immune responsive cells, or about 1×10⁶ to about 1×10⁷ immune responsive cells. However, depending on the location, source, identity, degree and severity of the cancer, the age and physical condition of an individual to be treated, and the like, the number of CAR immune responsive cells administered to the individual will vary within a wide range. The doctor will finally determine the appropriate dosage to be used.

In some embodiments, chimeric antigen receptors are used to stimulate immune cell-mediated immune responses. For example, a T cell-mediated immune response is an immune response involving T cell activation. Activated antigen-specific cytotoxic T cells can induce apoptosis in target cells displaying foreign antigen epitopes on the surface, such as cancer cells displaying tumor antigens. In another embodiment, chimeric antigen receptors are used to provide anti-tumor immunities in mammals. A subject will develop anti-tumor immunities, due to the T cell-mediated immune response.

In some cases, the method for treating a subject with cancer may involve administering one or more immune effector cells of the present invention to the subject in need of treatment. The immune effector cells can bind tumor target molecules and induce the death of cancer cells. As described above, the present invention also provides a method for treating pathogen infection in an individual, which comprises administering to the individual a therapeutically effective amount of the immune effector cells of the present invention.

The frequency of administration of the immune responsive cells of the present invention will be based on factors, including the disease to be treated, the elements of the specific immune responsive cells, and the mode of administration. As described herein, the immune effector cells of the present application have improved viability, therefore they can not only be administered in a lower therapeutically effective amount as compared with similar immune effector cells that do not express exogenous IL12, but also can be administered in a lower frequency of administration to obtain at least similar, and preferably more significant therapeutic effects.

Combination with Anti-Tumor Drugs

In some embodiments, the immune effector cells of the present invention may be administered in combination with another therapeutic agent. In some embodiments, the other therapeutic agent is a chemotherapeutic agent. The chemotherapeutic drugs that can be used in combination with the immune effector cells of the present invention include but are not limited to mitotic inhibitors (vinca alkaloids), including vincristine, vinblastine, vindesine and novibine (TM) (vinorelbine), 5′-dehydrogen sulfide); topoisomerase I inhibitors, such as camptothecin compounds, including Camptosar™ (irinotecan HCL), Hycamtin™ (topotecan HCL) and other compounds derived from camptothecin and analoges thereof; podophyllotoxin derivatives, such as etoposide, teniposide and midoxizoz; alkylating agents, cisplatin, cyclophosphamide, nitrogen mustard, trimethylene thiophosphoramide, carmustine, busulfan, chlorambucil, briquiazine, uracil mustard, cloprofen, and dacarbazine; antimetabolites, including cytarabine, fluorouracil, methotrexate, mercaptopurine, azathioprine, and procarbazine; antibiotics, including but not limited to doxorubicin, bleomycin, dactinomycin, daunorubicin, mycinomycin, mitomycin, sarcomycin C, and doxorubicin; and other chemotherapy drugs, including but not limited to anti-tumor antibodies, dacarbazine, azacytidine, amsacam, melphalan, ifosfamide and mitoxantrone.

In some embodiments, chemotherapeutic drugs that can be used in combination with the immune effector cells of the present invention include, but are not limited to, anti-angiogenic agents, including anti-VEGF antibodies (including humanized and chimeric antibodies, anti-VEGF aptamers and antisense oligonucleotides) and other angiogenesis inhibitors, such as angiostatin, endostatin, interferon, retinoic acid, and tissue inhibitors of metalloproteinase-1 and -2.

Kit

The present invention also provides a kit containing the immune effector cells of the present invention. The kit can be used to treat or prevent cancer, pathogen infection, immune disorder, or allogeneic transplantation. In one embodiment, the kit may include a therapeutic or preventive composition containing an effective amount of immune effector cells of one or more unit dosage forms.

In some embodiments, the kit includes a sterile container that can contain a therapeutic or preventive composition.

In some cases, the kit may include about 1×10⁴ cells to about 1×10⁶ cells. In some cases, the kit may include at least about 1×10⁵ cells, at least about 1×10⁶ cells, at least about 1×10⁷ cells, at least about 4×10⁷ cells, at least about 5×10⁷ cells, at least about 6×10⁷ cells, at least about 6×10⁷ cells, 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 6×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 9×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 8×10¹¹ cells, at least about 9×10¹¹ cells, or at least about 1×10¹² cells. For example, about 5×10¹⁰ cells can be included in the kit.

In some cases, the kit may include allogeneic cells. In some cases, the kit can include cells with genomic modifications. In some cases, the kit may contain “ready-for-use” cells. In some cases, the kit can include cells that can be expanded for clinical use. In some cases, the kit may contain contents for research purposes.

Autologous lymphocyte infusion can be used for treatment. Autologous peripheral blood mononuclear cells (PBMC) can be collected from a patient in need of treatment, and T cells can be activated and expanded using methods described herein and known in the art, and then injected into the patient. In other cases, allogeneic cells can be used to treat patients.

The methods disclosed herein can include transplantation. Transplantation can refer to the adoptive transplantation of cell products. The transplantation can be autologous transplantation, allogeneic transplantation, xenotransplantation or any other transplantation. For example, the transplantation can be a xenotransplantation. The transplantation can also be an allogeneic transplantation.

### Example 1. Preparation of GPC3-CAR-T Cells/IL12-GPC3-CAR T Cells

1. Construction of Plasmid

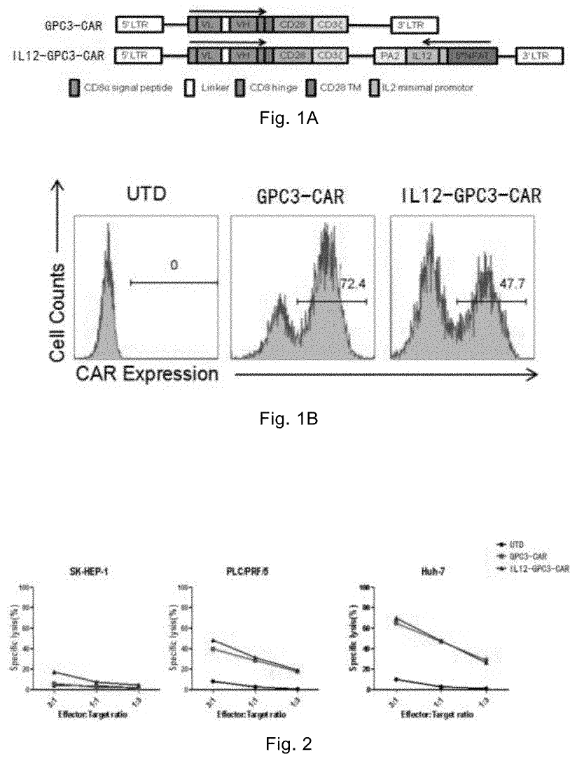

The chimeric antigen receptor used in this example is a second-generation of chimeric antigen receptor, and the encoding nucleotide sequence of the scFv targeting the extracellular domain of the GPC3 receptor is shown in SEQ ID NO:1, which also comprises the transmembrane domain of CD28, the intracellular domain of CD28, and CD3ζ. Plasmids for GPC3-CAR-T cells and IL12-GPC3-CAR T cells were constructed, respectively (referring to FIG. 1A), as follows:

The GPC3-CAR-T sequence consists of CD8α signal peptide (SEQ ID NO: 2), scFv targeting GPC3 (SEQ ID NO: 1), hinge region of CD8 (SEQ ID NO: 3), CD28 transmembrane region (SEQ ID NO: 6) and intracellular signaling domain (SEQ ID NO: 4) as well as intracellular segment CD3ζ of CD3 (SEQ ID NO: 5).

For the plasmid of IL12-GPC3-CAR, the NFAT6-IL12 sequence was inserted into the GPC3-CAR-T plasmid to construct a lentiviral plasmid expressing a second-generation of chimeric antigen receptor of GPC3 and IL12. The NFAT6-IL12 sequence is composed of 6*NFAT binding motif (SEQ ID NO: 7), IL2 minimal promoter (SEQ ID NO: 8), IL12 signal peptide and IL12 p40 (SEQ ID NO: 10), (G45)3 Linker (SEQ ID NO: ID NO: 9), IL12 p35 (SEQ ID NO: 11), PA2 (SEQ ID NO: 20).

2. Lentivirus Packaging, Virus Concentration and Titer Determination

a. Lentivirus Packaging

1) 293T cells cultured to the 6^{th} to 10^{th} passage at a density of 5×10⁶ were inoculated in a petri dish, and cultured overnight at 37° C., 5% CO2, and the medium was DMEM containing 10% fetal bovine serum (Gibico);

2) target gene plasmids GPC3-CAR-T and IL12-GPC3-CAR T (5.4 μg) and packaging plasmids pRsv-REV (6.2 μg), RRE-PMDLg (6.2 μg), and Vsvg (2.4 μg) were dissolved into 800 μL of blank DMEM culture medium, and mixed well, so as to obtain a plasmid mixture;

3) 60 μg of PEI (1 μg/μl) was dissolved in 800 μl of serum-free DMEM culture medium, and incubated at room temperature for 5 minutes to obtain a PEI mixture;

4) the plasmid mixture was added into the PEI mixture, mixed, and incubated at room temperature for 20 minutes to form a transfection complex;

5) 1.6 ml of the transfection complex was added dropwise to a 10 cm petri dish containing 11 ml of DMEM medium. After 4-5 h hours, the medium of the transfected 293T cells was changed to DMEM medium with 10% FBS, and incubated at 37° C. for 72 h, and the virus supernatant was collected.

b. Lentivirus Concentration

1) preparation of SXPEG8000 NaCl: 8.766 g of NaCl and 50 g of PEG8000 were weighted and dissolved in 200 ml of Milli-Q pure water; sterilized at 121° C. for 30 min and stored at 4° C.;

2) a 0.45 μm filter was used to filter the virus supernatant, and 7.5 ml of 5×PEG-8000 NaCl stock solution was added into every 30 ml of the filtered virus initial solution; mixed once every 20-30 min for a total of 3-5 times; placed at 4° C. overnight. After centrifugation, the supernatant was aspirated and discarded, the precipitate was left stood and the remaining liquid was removed; an appropriate amount of lentivirus lysis solution was added to dissolve the lentivirus precipitation; and the concentrated virus suspension was stored at −80° C.

The titer of lentivirus was detected by flow cytometry (preferably, the number of cells with a positive rate of 5-20%), the titer (U/mL) was calculate as=number of cells×positive rate/virus volume, and the titer of the concentrated virus were:

GPC3-CAR-T: 2.4×108 U/ml

IL12-GPC3-CAR T: 1×108 U/ml

3. Preparation of GPC3-CAR-T Cells and IL12-GPC3-CAR T Cells

1) anti-CD3 and CD28 antibody magnetic beads (Invitrogen) were used for T lymphocyte activation;

2) The day before infection, a 24-well plate was coated with RetroNectin: 380 μl of 5 μg/ml RetroNectin solution (PBS) was added to each well, and incubated overnight at 4° C.;

3) the RetroNectin solution (PBS) in the 24-well plate was discarded, the plate was washed twice with 1 ml PBS; the activated T cells were inoculated in a 24-well plate coated with retronectin at 5×10⁵ cells per well and 500 μl of culture medium; concentrated lentivirus was added into PBMC cells at MOI=15, centrifuged at 32° C., 1800 rpm, for 40 min, then transferred to a cell incubator;

4) Expansion culture: the infected cells were passaged every other day at a density of 5×10⁵/mL, and the lymphocyte culture medium was supplemented with recombinant human IL-2 at a final concentration of 500 U/mL.

GPC3-CAR-T cells/IL12-GPC3-CAR T positive rate (purity) were determined by using conventional flow cytometry (the determination method is a conventional method in the art, such as the method disclosed in Kowolik et al., 2006) The results are shown in FIG. 1B, in which UTD is T cells not transfected with CAR, GPC3-CAR is T cells transfected with pRRLSIN.cPPT.EF-1α-9F2-28Z, and IL12-GPC3-CAR is T cells transfected with pRRLSIN.cPPT.EF-1α-9F2-28Z-NFAT6-IL12. The results show that both of 9F2-28Z/9F2-28Z-NFAT6-IL12 have a higher level of CAR expression on the surface.

### Example 2. Cytotoxicity Assay of CAR-T/IL12-GPC3-CAR T Cells Targeting GPC3

CytoTox 96 non-radioactive cytotoxicity detection kit (Promega) was used to detect cytotoxicity. Details can be found in the instructions of CytoTox 96 non-radioactive cytotoxicity detection kit.

1) Target cells: Huh-7, PLC/PRF/5, SK-HEP-1 cells were selected as target cells, among which Huh-7 cells and PLC/PRF/5 cells were GPC3-positive, and SK-HEP-1 cells were GPC3 negative;

2) Effector cells: UTD, GPC3-CAR-T and IL12-GPC3-CAR T cells were added at an effector target ratio of 3:1, 1:1 or 1:3;

The experimental results are shown in FIG. 2. Compared with the UTD group, both of GPC3-CAR-T/IL12-GPC3-CAR T showed stronger cytotoxicity in the in vitro toxicity experiment, and there was no significant difference. For GPC3-negative liver cancer cells, GPC3-CAR-T/IL12-GPC3-CAR T exhibited no enhanced killing effects.

### Example 3. Secretion of Cytokines of GPC3-CAR-T/IL12-GPC3-CAR T Cells

The UTD/GPC3-CAR-T/IL12-GPC3-CAR T were incubated with the liver cancer cell line Huh-7, PLC/PRF/5, SK-HEP-1 cells at 1:1 for 24 hours, and then the supernatant was collected. The supernatant was detected by ELISA for secretion levels of cytokines.

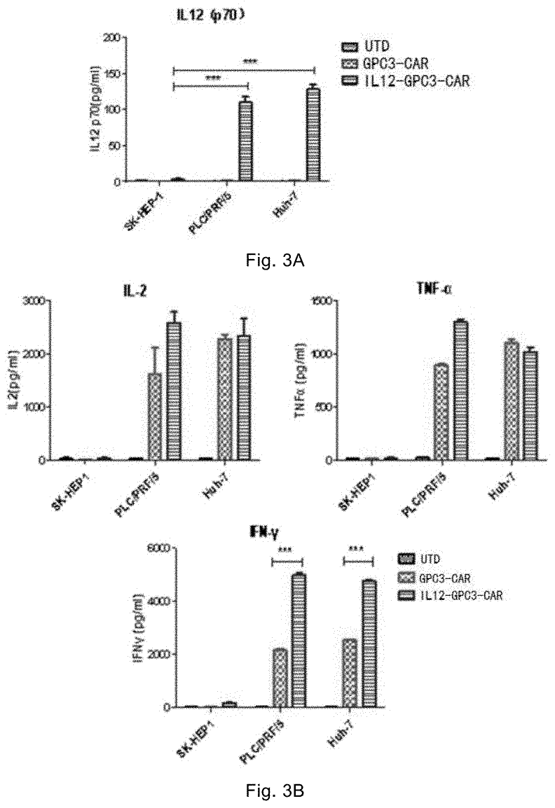

The samples for detecting TNF-α and IL12 were not diluted, and the samples for detecting IL2 and IFN-γ were diluted 20 times and 25 times respectively. Double-antibody sandwich enzyme-linked immunosorbent detection technology was used for ELISA kit. The results are shown in FIGS. 3A and 3B. FIG. 3A shows that a higher level of IL12 secretion was detected for IL12-GPC3-CAR T, when co-incubated with GPC3+tumor cells Huh-7, PLC/PRF/5, and IL12 was almost undetectable, when co-incubated with GPC3-tumor cells SK-HEP-1, which means that the CAR-T cell can only secrete IL12 when it recognizes GPC3. FIG. 3B shows the release of cytokines IL-2, TNF-α, IFN-γ when UTD/GPC3-CAR-T/IL12-GPC3-CAR T were incubated with tumor cells for 24 hours, showing that IL12-GPC3-CAR T obviously secreted more IFN-γ.

### Example 4. Therapeutic Effects of GPC3-CAR-T/IL12-GPC3-CAR T on Huh-7 Subcutaneous Xenograft Tumors with Medium and High Endogenous Expression of GPC3

1. NOD/SCID mice were inoculated with Huh-7 subcutaneous transplantation tumor Huh-7 cells were subcutaneously inoculated, 2×10⁶/mouse, and the tumor volume reached 300 mm³ after about 10 days. The mice were randomly grouped (n=6);

2. Preparation and expansion of UTD/GPC3-CAR-T/IL12-GPC3-CAR T cells;

3. UTD/GPC3-CAR-T/IL12-GPC3-CAR T cell adoptive immunotherapy for Huh-7 transplanted tumors

1) for subcutaneously transplanted tumors, cyclophosphamide (100 mg/kg) was intraperitoneally injected within 10 days;

2) On Day 10, the tumor volume of NOD/SCID mice was measured and the mice were randomly grouped. The mice with subcutaneous transplanted tumor were divided into 3 groups, including: UTD group, GPC3-CAR-T group, IL12-GPC3-CAR T group (n=6);

3) On Day 12, NOD/SCID mice were subjected to adoptive immunotherapy after being grouped. Low-dose of CAR-T were given through tail vein injection at a dose of 1×10⁷ cells/mouse;

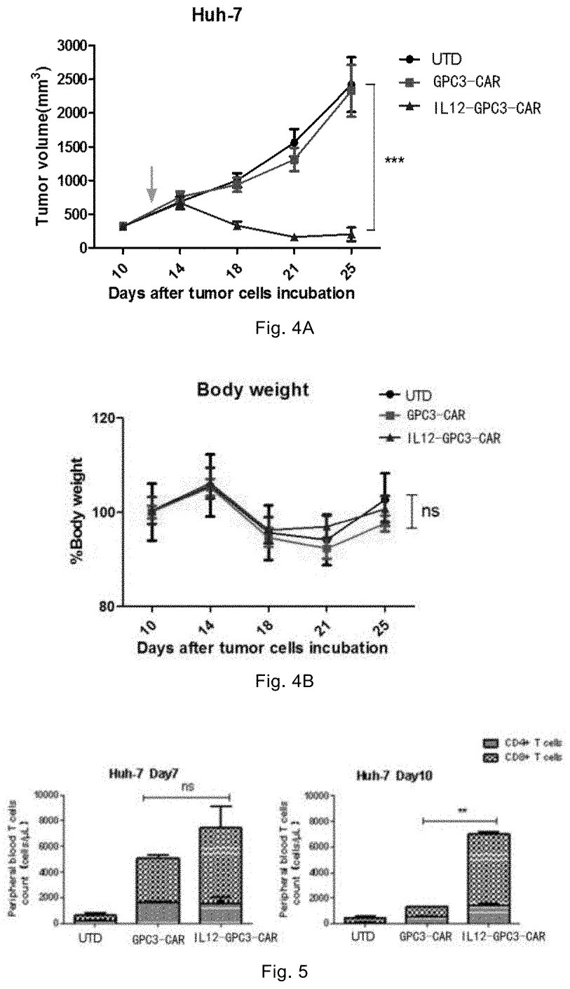

4) the volume of Huh-7 subcutaneously transplanted tumor was measured every 3-4 days, changes in the tumor volume in each group of mice were recorded, and the growth curves of tumor volume over time were plotted. The results are shown in FIG. 4A and FIG. 4B.

FIG. 4A shows the growth of Huh-7 subcutaneously xenografted tumors. After CAR-T cells were administered for 2 days, IL12-GPC3-CAR T showed significant tumor suppression and killing effects, while in the GPC3-CAR-T group, the tumors showed an increasing trend starting from the 14^{th} day, which is not significantly different from the blank control group. It shows that IL12-GPC3-CAR T still shows excellent killing activity for tumors that cannot be controlled by GPC3-CAR.

FIG. 4B shows that after IL12-GPC3-CAR T was administered, there was no significant difference in the body weight of mice in the GPC3-CAR-T group and the blank control group, indicating that although IL12-GPC3-CAR T showed significant anti-tumor effects, it did not cause side effects of treatment-related weight loss. In addition, the observation of mice in the experimental group showed that, after IL12-GPC3-CAR T were administered, the mice did not show obvious side effects.

Blood was taken from the tail vein on the 7^{th} day and 10^{th} day after the CAR-T injection, and the survival of CAR-T cells was detected by flow cytometry. The results are shown in FIG. 5, which shows that on the 7^{th} day, the survival number of CAR-T cells in the GPC3-CAR-T group was lower than 6000 cells/μL, and reached nearly 8000 cells/μL in the IL12-GPC3-CAR T group. On the 10^{th} day, CAR-T cells in the GPC3-CAR-T group were significantly reduced (lower than 2000 cells/4), while high cell survival was maintained in IL12-GPC3-CAR T group. Compared with the data of IL12-GPC3-CAR T on day 7, there is little difference. Compared with the GPC3-CAR-T group, in the IL12-GPC3-CAR T group, the number of CAR-T cells in peripheral blood is at least 75% higher (calculation method: IL12-GPC3-CAR T group-GPC3-CAR-T group/IL12-GPC3-CAR T group), indicating that the in vivo survival of T cells for IL12-GPC3-CAR T is far superior to CAR-T products not comprising IL12.

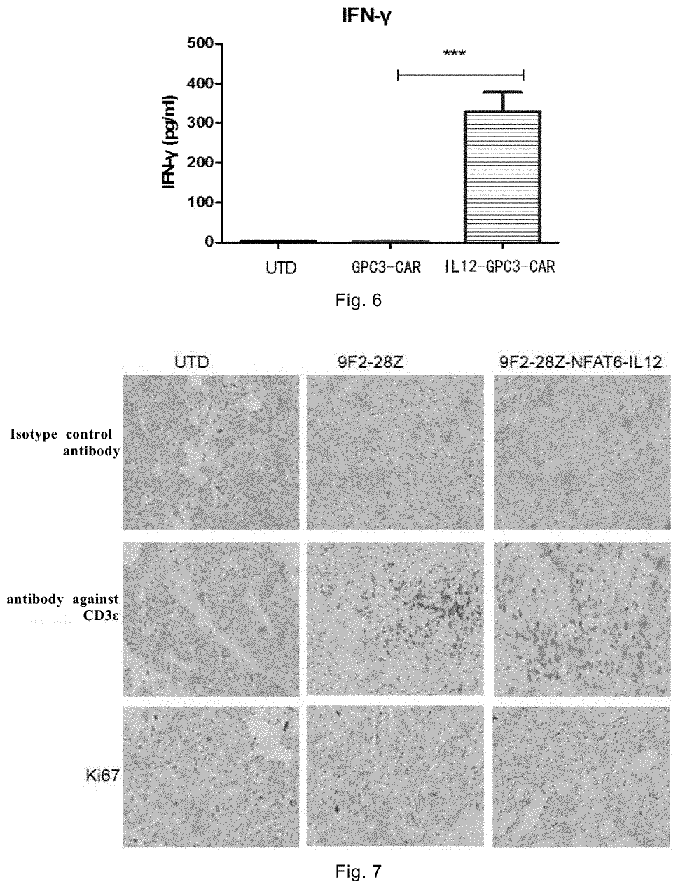

The peripheral blood of mice was collected on Day 10, and the serum samples were tested for IFN-γ by ELISA. The detection method was the same as that in Example 3. The results are shown in FIG. 6, which shows that the IFN-γ in the blood of mice in the IL12-GPC3-CAR T group was much higher than that in the GPC3-CAR-T group.

At the end of the experiment, the subcutaneous tumors were taken, and the infiltrating T cells and Ki67 in the tumor tissues were tested by immunohistochemistry. The results are shown in FIG. 7. The CAR-T infiltration in IL12-GPC3-CAR T group was significantly more than that in GPC3-CAR-T group. In the IL12-GPC3-CAR T group, the nuclear proliferation of Marker Ki67 was significantly less than that of the other two groups. The decrease in Ki67 indicates that the proliferation of cells in the tumor is reduced, which means that IL12-GPC3-CAR T can effectively eliminate the GPC3+tumor cells and inhibit the proliferation of tumor cells.

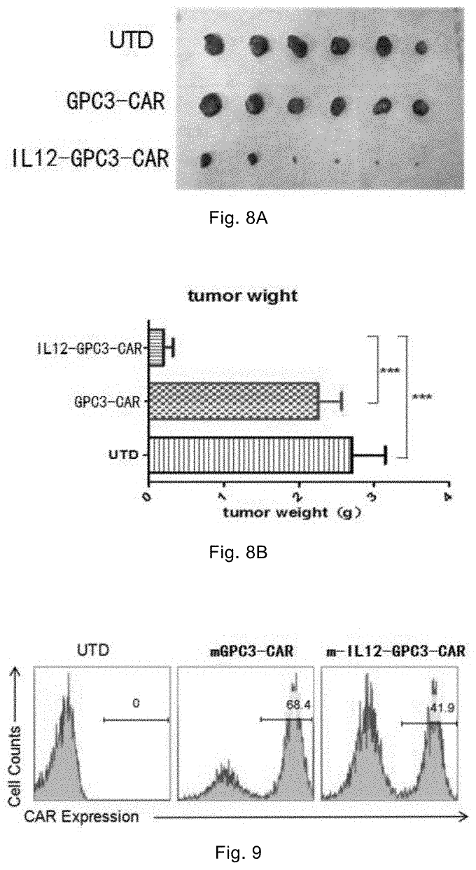

At the end of the experiment, the mice were euthanized and their subcutaneous tumors were weighed. As shown in FIG. 8A and FIG. 8B, the tumor volume and weight of the IL12-GPC3-CAR T group were significantly lower than those of the second-generation CAR-T group at the end of the experiment, and there are significant statistical differences.

### Example 5. Preparation of Mouse CAR-T (mGPC3-CAR) and m-IL12-GPC3-CAR T (Mouse CAR-IL12) Cells

In order to further verify the role of IL12 in mobilizing innate immunity in immune-sound mice, mouse-derived CAR-T were prepared.

1. Construction of Plasmid

Gene sequences of the mouse CD8α signal peptide (SEQ ID NO: 12), the anti-GPC3 monoclonal antibody (SEQ ID NO: 1), the mouse CD8α hinge region and transmembrane region (SEQ ID NO: 13), the mouse CD28 intracellular domain (SEQ ID NO: ID NO: 14) and the mouse CD3ζ intracellular domain (SEQ ID NO: 15) were connected in sequence, and the GPC3-CAR-T gene fragments were obtained by in vitro gene synthesis. The IRES-GFP fragment in the retroviral vector MSCV-IRES-GFP was replaced with Mlu I and Sal I restriction sites to obtain the recombinant vector MSCV-GPC3-CAR-T. For mouse CAR-IL12 (m-IL12-GPC3-CAR T), the mNFAT6-mIL12 sequence was inserted into the MSCV-GPC3-CAR-T plasmid to construct a plasmid expressing a second-generation of chimeric antigen receptor of GPC3 and mIL12, MSCV-IL12-GPC3-CAR T. The mNFAT6-mIL12 sequence is composed of 6*mNFAT binding motif (SEQ ID NO: 16), mIL2 minimal promoter (SEQ ID NO: 17), mIL12 signal peptide and mIL12 p40 (SEQ ID NO: 18), (SG4)3 Linker (SEQ ID NO: ID NO: 9), mIL12 p35 (SEQ ID NO: 19), PA2 (SEQ ID NO: 20).

2. Packaging of Retrovirus

1) 293T cells cultured to the 6^{th} to 10^{th} passage were inoculated at a density of 5×106 in a 10 cm petri dish, and cultured overnight at 37° C., 5% CO₂, and the medium is DMEM containing 10% fetal bovine serum (Gibico);

2) 9 μg of MSCV-GPC3-CAR-T or MSCV-GPC3-CAR-T-mNFAT6-mIL12 and 9 μg of packaging plasmid pCL-Eco were dissolved into 800 μL of serum-free DMEM culture medium and mixed to obtain a plasmid mixture; and 54 μg of PEI (1 μg/μl) was dissolved in 800 μl of serum-free DMEM medium, mixed well, and incubated at room temperature for 5 minutes to obtain a PEI mixture;

3) a plasmid mixture was added to a PEI mixture and mixed, and incubated at room temperature for 20 minutes to form a transfection complex;

4) 1.6 ml of the transfection complex was added dropwise to a 10 cm petri dish containing 11 ml of DMEM medium. After 4-5 h hours, the medium of the transfected 293T cells was exchanged with 10% FBS DMEM medium, and incubated at 37° C. for 72 h. The virus supernatant was collected to obtain the retrovirus carrying mGPC3-CAR-T/mGPC3-CAR-T-mNFAT6-mIL12.

4. Preparation of Mouse CAR-T Lymphocytes

Healthy Balb/c mice were taken and Cell isolation kit (STEMCELL Technologies) was used to isolate mouse spleen T lymphocytes according to the instructions.

The purified mouse CD3+T lymphocytes were added to Dynabeads Mouse T-activator CD3/CD28 (washed once with PBS) at a ratio of 1:1, and cultured in an incubator. The medium was RPMI 1640 complete medium for activation.

Mouse splenic T lymphocytes activated for 24 hours were added in a 48-well plate coated with retronectin at a cell number of 1×10⁶ per well. 1 ml of retrovirus of mGPC3-CAR-T/mGPC3-CAR-T-mNFAT6-mIL12 was added, the medium was supplemented to 2 mL. After centrifuged at 2000 g, 32° C. for 90 minutes, the cells were transferred an incubator for further culture. On the next day, the medium was exchanged to a fresh medium, and the cell density was adjusted to 5×10⁵/mL, and passaged every 2-3 days.

Assay on mCAR-T Cell Chimeric Antigen Receptor Expression:

1×10⁶ mCAR-T/mIL12-GPC3-CAR T cells were taken, divided into 2 portions, added in a 2 ml centrifuge tube, and centrifuged at 4° C., 400 g for 5 min. The supernatant was discarded, and the precipitate was washed twice with PBS. 50 μl of PE-SA (diluted 1:200) was added into cells in the control group and incubated on ice for 45 min, washed twice with PBS (2% NBS); and resuspended as a control. 50 μl of 1:50 diluted biotin-Goat anti Human IgG, F(ab′)₂ antibody was added into cells in the test group, incubated on ice for 45 min; and washed twice with PBS (2% NBS); and 50 μl of PE-SA (diluted 1:200) antibody was added and incubated on ice for 45 min.

2 ml PBS (2% NBS) was added to resuspend the cells, and centrifuged at 4° C., 400 g for 5 minutes to discard the supernatant (repeated twice); 500 μl of PBS (2% NBS) was added, and FACS detection was performed. The results are shown in FIG. 9, which show that there is a higher expression level of CAR on the surface of m9F2-m28Z/m9F2-m28Z-mNFAT6-mIL12, and UTD is mT cells without being transfected with CAR.

### Example 6. Cytotoxicity Assay of mCAR-T/mIL12-GPC3-CAR T Cells Targeting GPC3

CytoTox 96 non-radioactive cytotoxicity detection kit (Promega) was used, and details can be found in the instructions of CytoTox 96 non-radioactive cytotoxicity detection kit.

1) Target cells: 50 μL of 2×10⁵/mL E0771-Parental, E0771-Parental-GPC3, E0771-Recurrent (a cell line isolated and obtained from tumor tissue, after E0771 were inoculated into mice and tumor formed), E0771-Recurrent-GPC3 cells were inoculated into 96 well plate, respectively;

2) Effector cells: UTD, mGPC3-m28Z and mGPC3-m28Z-mNFAT6-mIL12 cells were added at an effector target ratio of 3:1, 1:1 or 1:3;

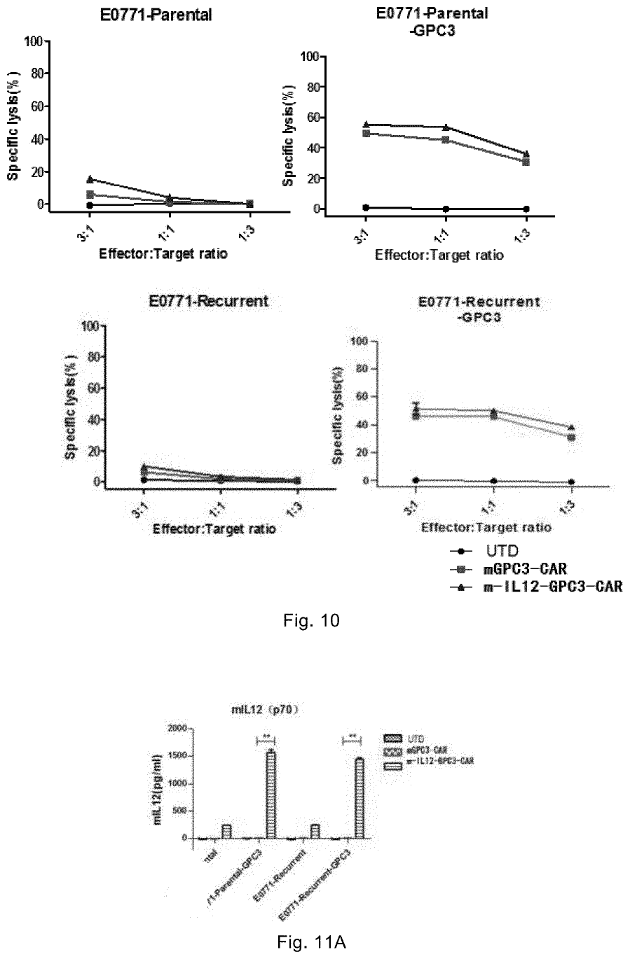

The experimental results are shown in FIG. 10. Compared with the UTD group, both of mGPC3-CAR and mGPC3-m28Z-mNFAT6-mIL12 (mIL12-GPC3-CAR T) showed stronger cytotoxicity in in vitro toxicity experiments, and there is no obvious difference. For GPC3-negative tumor cells, mGPC3-CAR and mIL12-GPC3-CAR T have no enhanced killing effects.

### Example 7. Secretion of Cytokines of mCAR-T/mIL12-GPC3-CAR T Cell

UTD/mGPC3-CAR/mIL12-GPC3-CAR T were co-incubated with E0771-Parental, E0771-Parental-GPC3, E0771-Recurrent, E0771-Recurrent-GPC3 cells at 1:1 for 24 hours. The supernatant was collected, and the supernatant was detect by ELISA for cytokine secretion level. The samples for detecting mTNF-α, mIL12, and mIL2 were not diluted, and the samples for detecting mIFN-γ were diluted 50 times. Double antibody sandwich enzyme-linked immunosorbent detection technology was used for the ELISA kit. Specific anti-mouse IL-2, TNF-α, IFN-γ, IL-12 monoclonal antibodies were pre-coated on high-affinity ELISA plates, respectively. The standard, sample to be tested and biotinylated detection antibody were added to the wells of a microtiter plate. After incubation, the mIL-2, mTNF-α, mIFN-γ, and mIL-12 present in a sample will be combined with solid-phase antibody and detection antibody, respectively. After unbound substances were washed and removed, horseradish peroxidase-labeled streptavidin (streptavidin-HRP) was added. The plate was washed and TMB-developped. The intensity of the color response is directly proportional to the concentration of the above-mentioned cytokines in the sample. A quench solution was added to stop the reaction, and the absorbance value was measured at 450 nm wavelength (reference wavelength of 570-630 nm).

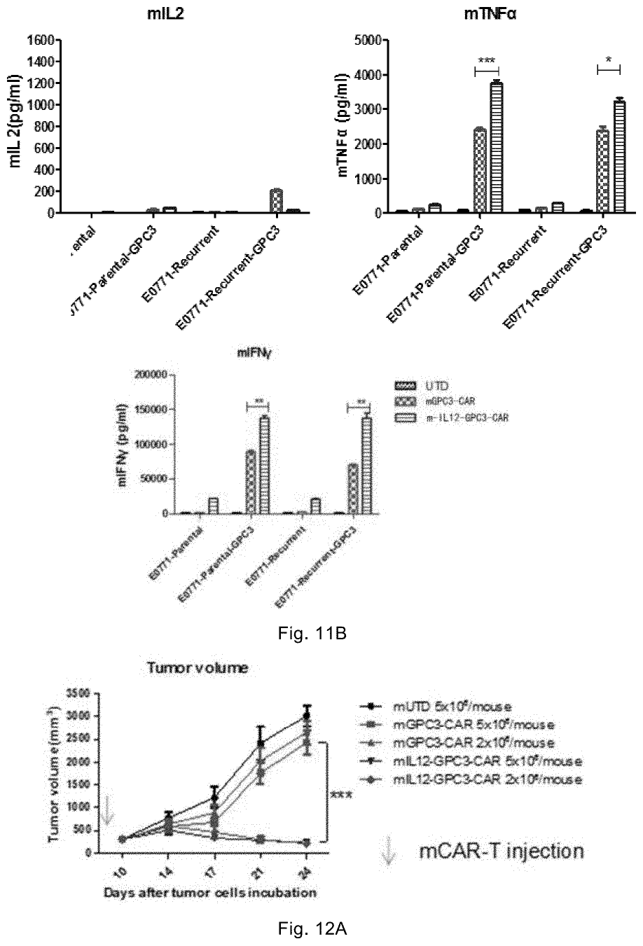

The results are shown in FIGS. 11A and 11B. FIG. 11A shows the secretion of mIL-12 when UTD, mGPC3-CAR, mGPC3-m28Z-mNFAT6-mIL12 were co-incubated with tumor cells for 24 hours, and when co-incubated with GPC3+tumor cells, mGPC3-m28Z-mNFAT6-mIL12 (mIL12-GPC3-CAR T) exhibited a higher secretion level of IL12, indicating that IL12 secretion in mice can also be inducible. FIG. 11B shows the release of cytokines mIL-2, mTNF-α, and mIFN-γ when UTD/mGPC3-m28Z/mIL12-GPC3-CAR T was co-incubated with tumor cells for 24 hours, and mIL12-GPC3-CAR T secreted significantly more IFN-γ.

### Example 8. Therapeutic Effects of mGPC3-CAR-T/mIL12-GPC3-T on E0771-GPC3 Subcutaneous Xenograft Tumor

1. C57BL/6 mice were inoculated with E0771-GPC3 subcutaneously transplanted tumor

E0771-GPC3 cells were subcutaneously inoculated at 1×10⁶/mouse, and the tumor volume reached 300 mm³ after about 10 days; and the mice were randomly grouped (n=6);

2. Preparation and expansion of mUTD/mGPC3-CAR-T/mIL12-GPC3-T cells;

3. Adoptive immunotherapy of MUTD/mGPC3-CAR-T/mIL12-GPC3-CAR-T cell on E0771-GPC3 transplanted tumor

1) on Day 10, E0771-GPC3 mice were measured for the volume of transplantation tumor and randomly grouped. The mice with subcutaneous transplanted tumor were divided into 5 groups, including: UTD group, mGPC3-CAR-T 5×10⁶ group, mGPC3-CAR-T 2×10⁶ group, mIL12-GPC3-T 5×10⁶ group, mIL12-GPC3-T 2×10⁶ group (n=6);

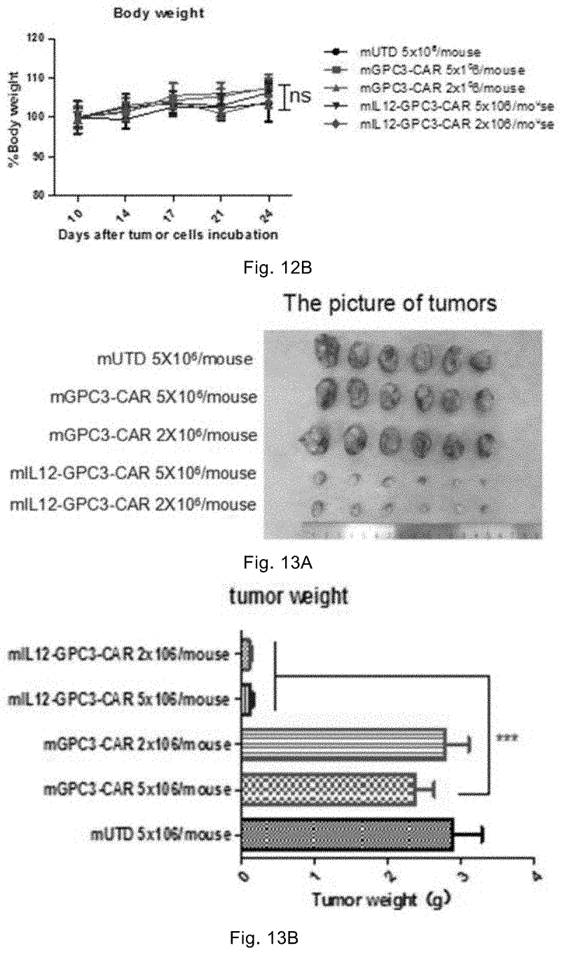

2) the volume of E0771-GPC3 subcutaneously transplanted tumor was measured every 3-4 days, changes in the tumor volume in each group of mice were recorded, and the growth curves of tumor volume over time was plotted. The results are shown in FIG. 12A and FIG. 12B.

FIG. 12A shows that the group administered with mIL12-GPC3-T 5×10⁶/animal through tail vein injection and the group administered with mIL12-GPC3-T 2×10⁶/animal through tail vein injection exhibited similar tumor-inhibiting effects at the end of the experiment, and both exhibited good tumor-inhibiting effects with a tumor-inhibiting rate over 90%, which shows that the ideal effects can still be achieved by infusing a small dosage of CAR-T for the large tumor load with the assistance of IL12. In contrast, in the group of mGPC3-CAR-T 5×10⁶/animal, the tumor volume is smaller than that in the mUTD group, however there is no statistical difference, which is due to the larger tumor volume (300 mm³) when CAR-T was injected.

FIG. 12B shows that after administration of mIL12-GPC3-T, there is no significant difference in the body weight of mice in the mGPC3-CAR-T group and the blank control group, indicating that IL12-GPC3-T exhibited significant anti-tumor effects, however side effects of treatment-related weight loss is not caused.

At the end of the experiment, the mice were euthanized and their subcutaneous tumors were weighed. The results are shown in FIG. 13A and FIG. 13B. At the end of the experiment, both of the tumor volume and the tumor weight in the mIL12-GPC3-T group were significantly lower than those of the second-generation of CAR-T group with a significant statistical difference.

The sequences involved in the present invention are summarized in the following table

| SEQ | | |
|---|---|---|
| ID | | |
| NO | Construct | Sequence |
| 1 | GPC3-scFv | Gaggtgcagctggtgcagagcggcgccgaggtgaagaagcccggcgccagcgtgaaggtgag |
| | | ctgcaAggccagcggctacaccttcagcgactacgagatgcactgggtgcggcaggcccccggc |
| | | cagggcctGgagtggatgggcgccatccaccccggcagcggcgacaccgcctacaaccagcgg |
| | | ttcaagggccggGtgaccatcaccgccgacaagagcaccagcaccgcctacatggagctgagca |
| | | gcctgcggagcgaggAcaccgccgtgtactactgcgcccggttctacagctacgcctactggggc |
| | | cagggcaccctggtgacCgtgagcgccggtggaggcggttcaggcggaggtggttctggcggtg |
| | | gcggatcggacatcgtgatgAcccagacccccctgagcctgcccgtgacccccggcgagcccgc |
| | | cagcatcagctgccggagcagccAgagcctggtgcacagcaacggcaacacctacctgcagtgg |
| | | tacctgcagaagcccggccagagcccCcagctgctgatctacaaggtgagcaaccggttcagcgg |
| | | cgtgcccgaccggttcagcggcagcggcAgcggcaccgacttcaccctgaagatcagccgggt |
| | | ggaggccgaggacgtgggcgtgtactactgcagccagagcatctacgtgccctacaccttcggcc |
| | | agggcaccaagctggagatcaaacgt |
| 2 | human CD8α | Atggccttaccagtgaccgccttgctcctgccgctggccttgctgctccacgccgccaggccg |
| | signal peptide | |
| 3 | human CD8α | Accacgacgccagcgccgcgaccaccaacaccggcgcccaccatcgcgtcgcagcccctgtcc |
| | hinge region | ctgcGcccagaggcgtgccggccagcggcggggggcgcagtgcacacgagggggctggactt |
| | | cgcctgtgat |
| 4 | human CD28 | AggagtaagaggagcaggctcctgcacagtgactacatgaacAtgactccccgccgccccgg |
| | intracellular | gccaacccgcaagcattaccagccctatgccccaccacgcgacttcgcagcctatcgctcc |
| | domain | |
| 5 | human CD3ζ | Agagtgaagttcagcaggagcgcagacgcccccgcgtaccagcagggccagaaccagctcta |
| | intracellular | taacgAgctcaatctaggacgaagagaggagtacgatgttttggacaagagacgtggccggga |
| | domain | ccctgagatGgggggaaagccgcagagaaggaagaaccctcaggaaggcctgtacaatgaac |
| | | tgcagaaagataagAtggcggaggcctacagtgagattgggatgaaaggcgagcgccggagg |
| | | ggcaaggggcacgatggccTttaccagggtctcagtacagccaccaaggacacctacgacgcc |
| | | cttcacatgcaggccctgccccctcgc |
| 6 | human CD28 | Ttttgggtgctggtggtggttggtggagtcctggcttgctatagcttgctagtaacagtggcc |
| | transmembrane | tttattattttctgggtg |
| | domain | |
| 7 | NFAT6 binding | Ggaggaaaaactgtttcatacagaaggcgtggaggaaaaactgtttcatacagaaggcgtggagg |
| | motif | aaaAactgtttcatacagaaggcgtcaattgtcctcgacggaggaaaaactgtttcatacagaaggc |
| | | gtggaggaaaaactgtttcatacagaaggcgtggaggaaaaactgtttcatacagaaggcgt |
| 8 | IL2 minimal | AcattttgacacccccataatatttaccagaattaacagtataaattgcatctcttgttcaagagttCcc |
| | promoter | tatcactctctttaatcactactcacagtaacctcaactcctg |
| 9 | (G₄S)₃ linker | Ggtggaggcggttcaggcggaggtggttctggcggtggcggatcg |
| 10 | IL12 signal | AtgtgtcaccagcagttggtcatctcttggttttccctggtttttctggcatctcccctcgtggccatAtg |
| | peptide and | ggaactgaagaaagatgtttatgtcgtagaattggattggtatccggatgcccctggagaaatggTgg |
| | IL12p40 | tcctcacctgtgacacccctgaagaagatggtatcacctggaccttggaccagagcagtgaggtcTta |
| | | ggctctggcaaaaccctgaccatccaagtcaaagagtttggagatgctggccagtacacctgtcaCaa |
| | | aggaggcgaggttctaagccattcgctcctgctgcttcacaaaaaggaagatggaatttggtccaCtga |
| | | tattttaaaggaccagaaagaacccaaaaataagacctttctaagatgcgaggccaagaattatTctgg |
| | | acgtttcacctgctggtggctgacgacaatcagtactgatttgacattcagtgtcaaaagcagCagagg |
| | | ctcttctgacccccaaggggtgacgtgcggagctgctacactctctgcagagagagtcagagGgga |
| | | caacaaggagtatgagtactcagtggagtgccaggaggacagtgcctgcccagctgctgaggagA |
| | | gtctgcccattgaggtcatggtggatgccgttcacaagctcaagtatgaaaactacaccagcagcttC |
| | | ttcatcagggacatcatcaaacctgacccacccaagaacttgcagctgaagccattaaagaattctcG |
| | | gcaggtggaggtcagctgggagtaccctgacacctggagtactccacattcctacttctccctgaca |
| | | Ttctgcgttcaggtccagggcaagagcaagagagaaaagaaagatagagtcttcacggacaagac |
| | | ctcAgccacggtcatctgccgcaaaaatgccagcattagcgtgcgggcccaggaccgctactatag |
| | | ctcatcttggagcgaatgggcatctgtgccctgcagt |
| 11 | IL12p35 | Agaaacctccccgtggccactccagacccaggaatgttcccatgccttcaccactcccaaaacct |
| | | gctGagggccgtcagcaacatgctccagaaggccagacaaactctagaattttacccttgcacttc |
| | | tgaagAgattgatcatgaagatatcacaaaagataaaaccagcacagtggaggcctgtttaccatt |
| | | ggaattaAccaagaatgagagagcctaaattccagagagacctattcataactaatgggagttgc |
| | | ctggcctcCagaaagacctcttttatgatggccctgtgccttagtagtatttatgaagacttgaagatg |
| | | taccaggTggagttcaagaccatgaatgcaaagcttctgatggatcctaagaggcagatattcta |
| | | gatcaaaacAtgctggcagttattgatgagctgatgcaggccctgaatttcaacagtg |
| | | agactgtgccacaaaaatcCtcccttgaagaaccggatttttataaaactaaaatcaagctctgcata |
| | | cttcttcatgctttcagaattcgggcagtgactattgatagagtgatgagctatctgaatgcttcctaa |
| 12 | mouse CD8α | Atggcctcaccgttgacccgctttctgtcgctgaacctgctgctgctgggtgagtcgattatcctgg |
| | signal peptide | ggagtggagaagct |
| 13 | mouse CD8α | Actactaccaagccagtgctgcgaactccctcacctgtgcaccctaccgggacatctcagcccc |
| | hinge region and | agagAccagaagattgtcggccccgtggctcagtgaaggggaccggattggacttcgcctgt |
| | transmembrane | gatatttacaTctgggcacccttggccggaatctgcgtggcccttctgctgtccttgatcatcact |
| | region | ctcatctgctaccacaggagccga |
| 14 | mouse CD28 | AatagtagaaggaacagactccttcaaagtgactacatgaacAtgactccccggaggcctgg |
| | intracellular | gctcactcgaaagccttaccagccctacgcccctgccagagactttgcagcgtaccgcccc |
| | domain | |
| 15 | mouse CD3ζ | Agcaggagtgcagagactgctgccaacctgcaggaccccaaccagctctacaatgagctcaa |
| | intracellular | tctaggGcgaagagaggaatatgacgtcttggagaagaagcgggctcgggatccagagatgg |
| | domain | gaggcaaacagcAgaggaggaggaacccccaggaaggcgtatacaatgcactgcagaaag |
| | | acaagatggcagaagcctacAgtgagatcggcacaaaaggcgagaggcggagaggcaagg |
| | | ggcacgatggcctttaccagggtctcagCactgccaccaaggacacctatgatgccctgcatat |
| | | gcagaccctggcc |
| 16 | mNFAT6 binding | Aagaggaaaatttgtttcatacagaaggcgttaagaggaaaatttgtttcatacagaaggcgttaa |
| | motif | gaGgaaaatttgtttcatacagaaggcgttaagaggaaaatttgtttcatacagaaggcgttaaga |
| | | ggaaaatttgtttcatacagaaggcgttaagaggaaaatttgtttcatacagaaggcgtt |
| 17 | mIL2 minimal | AacatcgtgacacccccatattatttttccagcattaacagtAtaaattgcctcccatgctgaaga |
| | promoter | gctgcctatcacccttgctaatcactcctcacagtgacctcaagtcct |
| 18 | mIL12 signal | Atgtgtcctcagaagctaaccatctcctggtttgccatcgttttgctggtgtctccactcatggcca |
| | peptide and | tGtgggagctggagaaagacgtttatgagtagaggtggactggactcccgatgcccctggag |
| | mIL12p40 | aaacagTgaacctcacctgtgacacgcctgaagaagatgacatcacctggacctcagaccag |
| | | agacatggagtcAtaggctctggaaagaccctgaccatcactgtcaaagagtttctagatgctg |
| | | gccagtacacctgccaCaaaggaggcgagactctgagccactcacatctgctgctccacaag |
| | | aaggaaaatggaatttggtccaCtgaaattttaaaaaatttcaaaaacaagactttcctgaagtgt |
| | | gaagcaccaaattactccggacggTtcacgtgctcatggctggtgcaaagaaacatggacttg |
| | | aagttcaacatcaagagcagtagcagttcCcctgactctcgggcagtgacatgtggaatggcg |
| | | tctctgtctgcagagaaggtcacactggaccaaaGggactatgagaagtattcagtgtcctgcc |
| | | aggaggatgtcacctgcccaactgccgaggagaccctgCccattgaactggcgttggaagc |
| | | acggcagcagaataaatatgagaactacagcaccagcttcttcatCagggacatcatcaaac |
| | | cagacccgcccaagaacttgcagatgaagcctttgaagaactcacaggtggAggtcagc |
| | | tgggagtaccctgactcctggagcactccccattcctacttctccctcaagttctttgaCgaa |
| | | tccagcgcaagaaagaaaagatgaaggagacagaggaggggtgtaaccagaaaggtgc |
| | | gttcctCgtagagaagacatctaccgaagtccaatgcaaaggcgggaatgtctgcgtgcaa |
| | | gctcaggatcgctattacaattcctcatgcagcaagtgggcatgtgttccctgcagggtccga |
| | | tcc |
| 19 | mIL12p35 | Agggtcattccagtctctggacctgccaggtgtcttagccagtcccgaaacctgctgaaga |
| | | ccacagaTgacatggtgaagacggccagagaaaaactgaaacattattcctgcactgctg |
| | | aagacatcgatcatgAagacatcacacgggaccaaaccagcacattgaagacctgtttac |
| | | cactggaactacacaagaacgagAgttgcctggctactagagagacttcttccacaacaa |
| | | gagggagctgcctgcccccacagaagacgtcTttgatgatgaccctgtgccttggtagca |
| | | tctatgaggacttgaagatgtaccagacagagttccaggCcatcaacgcagcacttcaga |
| | | atcacaaccatcagcagatcattctagacaagggcatgctggtggccAtcgatgagctg |
| | | atgcagtctctgaatcataatggcgagactctgcgccagaaacctcctgtgggagaAgc |
| | | agacccttacagagtgaaaatgaagctctgcatcctgcttcacgccttcagcacccgcgt |
| | | cgtgaccatcaacagggtgatgggctatctgagctccgcc |
| 20 | PA2 | AataaaatatctttattttcattacatctgtgtgttggttttTtgtgtgag |
| 21 | 9F2-28Z | EVQLVQSGAEVKKPGASVKVSCKASGYTFSDYEMHW |
| | | VRQAPGQGLEWMGAIHPGSGDTAYNQRFKGRVTITAD |
| | | KSTSTAYMELSSLRSEDTAVYYCARFYSYAYWGQGTL |
| | | VTVSAGGGGSGGGGSGGGGSDIVMTQTPLSLPVTPGEP |
| | | ASISCRSSQSLVHSNGNTYLQWYLQKPGQSPQLLIYKV |
| | | SNRFSGVPDRFSGSGSGTDFTLKISRVEAEDVGVYYCS |
| | | QSIYVPYTFGQGTKLEIKRTTTPAPRPPTPAPTIASQPLS |
| | | LRPEACRPAAGGAVHTRGLDFACDFWVLVV |
| | | VGGVLACYSLLVTVAFIIFWVRSKRSRLLHSDYMNMT |
| | | PRRPGPTRKHYQPYAPPRDFAAYRSRVKFSRSADAPA |
| | | YQQGQNQLYNELNLGRREEYDVLDKRRGRDPEMGGK |
| | | PQRRKNPQEGLYNELQKDKMAEAYSEIGMKGERRRG |
| | | KGHDGLYQGLSTATKDTYDALHMQALPPR |
| 22 | GPC3-BBZ | EVQLVQSGAEVKKPGASVKVSCKASGYTFSDYEMHW |
| | | VRQAPGQGLEWMGAIHPGSGDTAYNQRFKGRVTITA |
| | | DKSTSTAYMELSSLRSEDTAVYYCARFYSYAYWGQG |
| | | TLVTVSAGGGGSGGGGSGGGGSDIVMTQTPLSLPVTP |
| | | GEPASISCRSSQSLVHSNGNTYLQWYLQKPGQSPQLL |
| | | IYKVSNRFSGVPDRFSGSGSGTDFTLKISRVEAEDVG |
| | | VYYCSQSIYVPYTFGQGTKLEIKRTTTPAPRPPTPAP |
| | | TIASQPLSLRPEACRPAAGGAVHTRGLDFACDIYIWA |
| | | PLAGTCGVLLLSLVITLYCKRGRKKLLYIFKQPFMRP |
| | | VQTTQEEDGCSCRFPEEEEGGCELRVKFSRSADAPAY |
| | | QQGQNQLYNELNLGRREEYDVLDKRRGRDPEMGGK |
| | | PQRRKNPQEGLYNELQKDKMAEAYSEIGMKGERRR |
| | | GKGHDGLYQGLSTATKDTYDALHMQALPPR |
| 23 | GPC3-28BBZ | EVQLVQSGAEVKKPGASVKVSCKASGYTFSDYEMH |
| | | WVRQAPGQGLEWMGAIHPGSGDTAYNQRFKGRVT |
| | | ITADKSTSTAYMELSSLRSEDTAVYYCARFYSYAYW |
| | | GQGTLVTVSAGGGGSGGGGSGGGGSDIVMTQTPLS |
| | | LPVTPGEPASISCRSSQSLVHSNGNTYLQWYLQKPG |
| | | QSPQLLIYKVSNRFSGVPDRFSGSGSGTDFTLKISRV |
| | | EAEDVGVYYCSQSIYVPYTFGQGTKLEIKRTTTPAPR |
| | | PPTPAPTIASQPLSLRPEACRPAAGGAVHTRGLDFAC |
| | | DFWVLVVVGGVLACYSLLVTVAFIIFWVRSKRSRLL |
| | | HSDYMNMTPRRPGPTRKHYQPYAPPRDFAAYRSKR |
| | | GRKKLLYIFKQPFMRPVQTTQEEDGCSCRFPEEEEGG |
| | | CELRVKFSRSADAPAYQQGQNQLYNELNLGRREEYD |
| | | VLDKRRGRDPEMGGKPQRRKNPQEGLYNELQKDKM |
| | | AEAYSEIGMKGERRRGKGHDGLYQGLSTATKDTYD |
| | | ALHMQALPPR |
| 24 | GPC3-z | EVQLVQSGAEVKKPGASVKVSCKASGYTFSDYEMH |
| | | WVRQAPGQGLEWMGAIHPGSGDTAYNQRFKGRVT |
| | | ITADKSTSTAYMELSSLRSEDTAVYYCARFYSYAYW |
| | | GQGTLVTVSAGGGGSGGGGSGGGGSDIVMTQTPLS |
| | | LPVTPGEPASISCRSSQSLVHSNGNTYLQWYLQKPG |
| | | QSPQLLIYKVSNRFSGVPDRFSGSGSGTDFTLKISR |
| | | VEAEDVGVYYCSQSIYVPYTFGQGTKLEIKRrvkfsrsa |
| | | dApayqqgqnqlynelnlgrreeydvldkagrdpemggkpqrrKnpqeg |
| | | lynelqkdkmaeayseigmkgerrrgkghdglyqglstatkdtydalhmqalp |
| | | Pr |
| 25 | GPC3-scFv | EVQLVQSGAEVKKPGASVKVSCKASGYTFSDYEMH |
| | | WVRQAPGQGLEWMGAIHPGSGDTAYNQRFKGRVT |
| | | ITADKSTSTAYMELSSLRSEDTAVYYCARFYSYAYW |
| | | GQGTLVTVSAGGGGSGGGGSGGGGSDIVMTQTPLS |
| | | LPVTPGEPASISCRSSQSLVHSNGNTYLQWYLQKPG |
| | | QSPQLLIYKVSNRFSGVPDRFSGSGSGTDFTLKISRV |
| | | EAEDVGVYYCSQSIYVPYTFGQGTKLEIKR |
| 26 | amino acid | Mchqqlviswfslvflasplvaiwelkkdvyvveldwypdapgemvvltcd |
| | sequence of | tpeedgitWtldqssevlgsgktltiqvkefgdagqytchkggevlshsllllhk |
| | human IL12 | kedgiwstdilkDqkepknktfIrceaknysgrftcwwlttistdltfsvkssrg |
| | | ssdpqgqcgaatlsaErvrgdnkeyeysvecqedsacpaaeeslpievmv |
| | | davhklkyenytssffirdiikpdPpknlqlkplknsrqvevsweypdtwst |
| | | phsyfsltfcvqvqgkskrekkdrvftdlasAtvicrknasisvraqdryysss |
| | | wsewasvpcsggggsggggsggggsrnlpvatpdpgMfpclhhsqnllm |
| | | vsnmlqkarqtlefypctseeidheditkdktstveaclpleltkNesclnsrets |
| | | fitngsclasrktsfmmalclssiyedlkmyqvefktmnakllmdpkrQifld |
| | | qnmlavidelmqalnfnsetvpqkssleepdfyktkiklcillhafriravtidrv |
| | | msylnas* |
| 27 | amino acid | Mcpqkltiswfaivllvsplmamwelekdvyvvevdwtpdapgetvnltc |
| | sequence of | dtpeedditWtsdqrhgvigsglaltitykefldagqytchkggetlshshlllh |
| | mouce IL12 | kkengiwsteilkNfknktflkceapnysgrftcswlvqrnmdlkfnikssss |
| | | spdsravtcgmaslsaekvTldqrdyekysyscqedvtcptaeetlpielalea |
| | | rqqnkyenystsffirdiikpdppKnlqmkplknsqvevsweypdswstph |
| | | syfslkffvriqrkkekmketeegcnqkgaflVektstevqckggnvcvqaq |
| | | dryynsscskwacvpcrvsggggsggggsggggsrvipVsgparclsqsrn |
| | | llkttddmvktareklkhysctaedidheditrdqtstlktclpleLhknesclatr |
| | | etssttrgsclppqktslmmticlgsiyedlkmyqtefqainaalqnhnhqqiil |
| | | dkgmlvaidelmqslnhngetliqkppvgeadpyrvkmklcillhafstrvvt |
| | | inrvmgylssa* |
| 28 | IL12-9F2-28Z | Cgatggctccggtgcccgtcagtgggcagagcgcacatcgcccacagtcccc |
| | | gagaagtTggggggaggggtcggcaattgaaccggtgcctagagaaggtgg |
| | | cgcggggtaaactggGaaagtgatgtcgtgtactggctccgcctttttcccgag |
| | | ggtgggggagaaccgtatatAagtgcagtagtcgccgtgaacgttattttcgca |
| | | acgggtttgccgccagaacacaggTgtcgtgacgcggatccaggcctaagctt |
| | | acgcgtcctagcgctaccggtcgccaccatGgccttaccagtgaccgccttgct |
| | | cctgccgctggccttgctgctccacgccgccaggcCggaggtgcagctggtgc |
| | | agagcggcgccgaggtgaagaagcccggcgccagcgtgaagGtgagctgca |
| | | aggccagcggctacaccttcagcgactacgagatgcactgggtgcggcaGgcc |
| | | cccggccagggcctggagtggatgggcgccatccaccccggcagcggcgaca |
| | | ccgCctacaaccagcggttcaagggccgggtgaccatcaccgccgacaagag |
| | | caccagcaccGcctacatggagctgagcagcctgcggagcgaggacaccgc |
| | | cgtgtactactgcgcccgGttctacagctacgcctactggggccagggcaccct |
| | | ggtgaccgtgagcgccggtggagGcggttcaggcggaggtggttctggcggt |
| | | ggcggatcggacatcgtgatgacccagaccCccctgagcctgcccgtgaccc |
| | | ccggcgagcccgccagcatcagctgccggagcagccaGagcctggtgcaca |
| | | gcaacggcaacacctacctgcagtggtacctgcagaagcccggccAgagcc |
| | | cccagctgctgatctacaaggtgagcaaccggttcagcggcgtgcccgaccg |
| | | gTtcagcggcagcggcagcggcaccgacttcaccctgaagatcagccgggt |
| | | ggaggccgaGgacgtgggcgtgtactactgcagccagagcatctacgtgccc |
| | | tacaccttcggccaggGcaccaagctggagatcaaacgtaccacgacgccag |
| | | cgccgcgaccaccaacaccggcgCccaccatcgcgtcgcagcccctgtccc |
| | | tgcgcccagaggcgtgccggccagcggcgggGggcgcagtgcacacgag |
| | | ggggctggacttcgcctgtgatttttgggtgctggtggtggTtggtggagtcct |
| | | ggcttgctatagcttgctagtaacagtggcctttattattttctggGtgaggagta |
| | | agaggagcaggctcctgcacagtgactacatgaacatgactccccgccgC |
| | | cccgggccaacccgcaagcattaccagccctatgccccaccacgcgactt |
| | | cgcagcctAtcgctccagagtgaagttcagcaggagcgcagacgccccc |
| | | gcgtaccagcagggccagAaccagctctataacgagctcaatctaggacg |
| | | aagagaggagtacgatgttttggacaaGagacgtggccgggaccctgaga |
| | | tggggggaaagccgcagagaaggaagaaccctcaggAaggcctgtaca |
| | | atgaactgcagaaagataagatggcggaggcctacagtgagattgggAtg |
| | | aaaggcgagcgccggaggggcaaggggcacgatggcctttaccagggtc |
| | | tcagtacAgccaccaaggacacctacgacgcccttcacatgcaggccctg |
| | | ccccctcgctaggtcgAcaatcaacctctggattacaaaatttgtgaaagat |
| | | tgactggtattcttaactatgttGctccttttacgctatgtggatacgctgcttta |
| | | atgcctttgtatcatgctattgcttcCcgtatggctttcattttctcctccttgtat |
| | | aaatcctggttgctgtctctttatgaggAgagtggcccgttgtcaggcaacg |
| | | tggcgtggtgtgcactgtgtttgctgacgcaaccCccactggttggggcattg |
| | | ccaccacctgtcagctcctaccgggactttcgctttcccCctccctattgccac |
| | | ggcggaactcatcgccgcctgccttgcccgctgctggacaggggCtcggct |
| | | gttgggcactgacaattccgtggtgttgtcggggaagctgacgtcctttccaT |
| | | ggctgctcgcctgtgagccacctggattctgcgcgggacgtccttctgctacg |
| | | tcccTtcggccctcaatccagcggaccttccttcccgcggcctgctgccggc |
| | | tctgcggcctcTtccgcgtcttcgccttcgccctcagacgagtcggatctccc |
| | | tttgggccgcctccccgCctggaattcgctagcctcgagctcacacaaaaa |
| | | accaacacacagatgtaatgaaaatAaagatattttattgcggccgctttag |
| | | gaagcattcagatagctcatcactctatcaatAgtcactgcccgaattctgaa |
| | | agcatgaagaagtatgcagagcttgattttagttttatAaaaatccggttcttc |
| | | aagggaggatttttgtggcacagtctcactgttgaaattcaggGcctgcat |
| | | cagctcatcaataactgccagcatgttttgatctagaaagatctgcctcttA |
| | | ggatccatcagaagctttgcattcatggtcttgaactccacctggtacatctt |
| | | caagtCttcataaatactactaaggcacagggccatcataaaagaggtctt |
| | | tctggaggccaggCaactcccattagttatgaaagaggtctctctggaattt |
| | | aggcaactctcattcttggtTaattccaatggtaaacaggcctccactgtgc |
| | | tggttttatatttgtgatatcttcatGatcaatctcttcagaagtgcaagggta |
| | | aaattctagagtttgtctggccttctggagcAtgttgctgacggccctcagc |
| | | aggttttgggagtggtgaaggcatgggaacattcctggGtctggagtggc |
| | | cacggggaggtttctagatccgccgccacccgacccaccaccgcccgA |
| | | gccaccgccaccactgcagggcacagatgcccattcgctccaagatgag |
| | | ctatagtagCggtcctgggcccgcacgctaatgctggcatttagcggcag |
| | | atgaccgtggctgaggtCttgtccgtgaagactctatctttcttttctctcttgc |
| | | tcttgccctggacctgaacgcAgaatgtcagggagaagtaggaatgtgga |
| | | gtactccaggtgtcagggtactcccagctgAcctccacctgccgagaattc |
| | | tttaatggcttcagctgcaagttcttgggtgggtcaggTttgatgatgtccct |
| | | gatgaagaagctgctggtgtagtfficatacttgagcttgtgaaCggcatcc |
| | | accatgacctcaatgggcagactctcctcagcagctgggcaggcactgtc |
| | | cTcctggcactccactgagtactcatactccttgagtcccctctgactctct |
| | | ctgcagaGagtgtagcagctccgcacgtcaccccttgggggtcagaag |
| | | agcctctgctgcttagaCactgaatgtcaaatcagtactgattgtcgtcag |
| | | ccaccagcaggtgaaacgtccagaAtaattcttggcctcgcatcttagaa |
| | | aggtcttatttttgggttctttctggtcctttAaaatatcagtggaccaaattcc |
| | | atcttcattttgtgaagcagcaggagcgaatggcTtagaacctcgcctcc |
| | | tttgtgacaggtgtactggccagcatctccaaactctttgacTtggatggtc |
| | | agggttttgccagagcctaagacctcactgctctggtccaaggtccagGt |
| | | gataccatcttcttcaggggtgtcacaggtgaggaccaccatttctccagg |
| | | ggcatCcggataccaatccaattctacgacataaacatcificttcagttc |
| | | ccatatggccacGaggggagatgccagaaaaaccagggaaaacca |
| | | agagatgaccaactgctggtgacacAtggtggcgaccggtagcgcta |
| | | ggtcatatgcaggagttgaggttactgtgagtagtgAttaaagagagtg |
| | | atagggaactcttgaacaagagatgcaatttatactgttaattctGgaaaa |
| | | atattatgggggtgtcaaaatgtcccgggacaattgacgccttctgtatga |
| | | aAcagtttttcctccacgccttctgtatgaaacagtttttcctccacgccttc |
| | | tgtatgAaacagttfficctccgtcgaggacaattgacgccttctgtatgaa |
| | | acagtttttcctCcacgccttctgtatgaaacagtttttcctccacgccttctg |
| | | tatgaaacagtttttcCtcc |
| 29 | IL12-9F2-BBZ | Cgatggctccggtgcccgtcagtgggcagagcgcacatcgcccacagtc |
| | | cccgagaagTtggggggaggggtcggcaattgaaccggtgcctagaga |
| | | aggtggcgcggggtaaactGggaaagtgatgtcgtgtactggctccgcc |
| | | tttttcccgagggtgggggagaaccgtaTataagtgcagtagtcgccgtg |
| | | aacgttctttttcgcaacgggtttgccgccagaacaCaggtgtcgtgacgcg |
| | | gatccaggcctaagcttacgcgtcctagcgctaccggtcgccAccatggc |
| | | cttaccagtgaccgccttgctcctgccgctggccttgctgctccacgccgC |
| | | caggccggaggtgcagctggtgcagagcggcgccgaggtgaagaag |
| | | cccggcgccagCgtgaaggtgagctgcaaggccagcggctacacctt |
| | | cagcgactacgagatgcactggGtgcggcaggcccccggccagggc |
| | | ctggagtggatgggcgccatccaccccggcagcgGcgacaccgccta |
| | | caaccagcggttcaagggccgggtgaccatcaccgccgacaagagCa |
| | | ccagcaccgcctacatggagctgagcagcctgcggagcgaggacaccg |
| | | ccgtgtacTactgcgcccggttctacagctacgcctactggggccagggc |
| | | accctggtgaccgtgaGcgccggtggaggcggttcaggcggaggtggt |
| | | tctggcggtggcggatcggacatcgtGatgacccagacccccctgagcc |
| | | tgcccgtgacccccggcgagcccgccagcatcagcTgccggagcagc |
| | | cagagcctggtgcacagcaacggcaacacctacctgcagtggtaccTg |
| | | cagaagcccggccagagcccccagctgctgatctacaaggtgagcaac |
| | | cggttcagCggcgtgcccgaccggttcagcggcagcggcagcggca |
| | | ccgacttcaccctgaagatcAgccgggtggaggccgaggacgtggg |
| | | cgtgtactactgcagccagagcatctacgtgcCctacaccttcggccag |
| | | ggcaccaagctggagatcaaacgtaccacgacgccagcgccGcgac |
| | | caccaacaccggcgcccaccatcgcgtcgcagcccctgtccctgcgcc |
| | | cagagGcgtgccggccagcggcggggggcgcagtgcacacgaggg |
| | | ggctggacttcgcctgtgAtttttgggtgctggtggtggttggtggagtcc |
| | | tggcttgctatagcttgctagtaacAgtggcctttattattttctgggtgaaa |
| | | cggggcagaaagaaactcctgtatatattcAaacaaccatttatgagacc |
| | | agtacaaactactcaagaggaagatggctgtagctgccGatttccagaa |
| | | gaagaagaaggaggatgtgaactgagagtgaagttcagcaggagcg |
| | | cAgacgcccccgcgtaccagcagggccagaaccagctctataacg |
| | | agctcaatctaggaCgaagagaggagtacgatgttttggacaagag |
| | | acgtggccgggaccctgagatgggggGaaagccgcagagaagga |
| | | agaaccctcaggaaggcctgtacaatgaactgcagaaagaTaagat |
| | | ggcggaggcctacagtgagattgggatgaaaggcgagcgccgga |
| | | ggggcaagGggcacgatggcctttaccagggtctcagtacagccac |
| | | caaggacacctacgacgcccTtcacatgcaggccctgccccctcgc |
| | | taggtcgacaatcaacctctggattacaaaatTtgtgaaagattgactg |
| | | gtattcttaactatgttgctccttttacgctatgtggatacGctgctttaat |
| | | gcctttgtatcatgctattgcttcccgtatggctttcattttctcctCcttgt |
| | | ataaatcctggttgctgtctctttatgaggagttgtggcccgttgtcaggc |
| | | aAcgtggcgtggtgtgcactgtgtttgctgacgcaacccccactggtt |
| | | ggggcattgccAccacctgtcagctcctttccgggactttcgctttccc |
| | | cctccctattgccacggcggAactcatcgccgcctgccttgcccgct |
| | | gctggacaggggctcggctgttgggcactgaCaattccgtggtgttg |
| | | tcggggaagctgacgtcctttccatggctgctcgcctgtgttGccacc |
| | | tggattctgcgcgggacgtccttctgctacgtcccttcggccctcaat |
| | | ccagCggaccttccttcccgcggcctgctgccggctctgcggcctc |
| | | ttccgcgtcttcgccttCgccctcagacgagtcggatctccctttggg |
| | | ccgcctccccgcctggaattcgctagccTcgagctcacacaaaaa |
| | | accaacacacagatgtaatgaaaataaagatattttattgcgGccgc |
| | | tttaggaagcattcagatagctcatcactctatcaatagtcactgccc |
| | | gaattcTgaaagcatgaagaagtatgcagagcttgattttagtttta |
| | | taaaaatccggttcttcaAgggaggatttttgtggcacagtctcact |
| | | gttgaaattcagggcctgcatcagctcatcAataactgccagcatg |
| | | ttttgatctagaaagatctgcctcttaggatccatcagaagctTtgc |
| | | attcatggtcttgaactccacctggtacatcttcaagtcttcataaat |
| | | actactaAggcacagggccatcataaaagaggtctttctggaggc |
| | | caggcaactcccattagttatGaaagaggtctctctggaatttagg |
| | | caactctcattcttggttaattccaatggtaaacAggcctccactg |
| | | tgctggttttatcttttgtgatatcttcatgatcaatctcttcagaaGt |
| | | gcaagggtaaaattctagagtttgtctggccttctggagcatgttg |
| | | ctgacggccctCagcaggttttgggagtggtgaaggcatggg |
| | | aacattcctgggtctggagtggccacggGgaggtttctagatc |
| | | cgccgccacccgacccaccaccgcccgagccaccgccacca |
| | | ctgCagggcacagatgcccattcgctccaagatgagctatagt |
| | | agcggtcctgggcccgcacGctaatgctggcatttttgcggc |
| | | agatgaccgtggctgaggtcttgtccgtgaagactcTatctttc |
| | | ttttctctcttgctcttgccctggacctgaacgcagaatgtcaggg |
| | | agaagTaggaatgtggagtactccaggtgtcagggtactccca |
| | | gctgacctccacctgccgagaAttctttaatggcttcagctgcaa |
| | | gttcttgggtgggtcaggtttgatgatgtccctgaTgaagaagct |
| | | gctggtgtagttttcatacttgagcttgtgaacggcatccaccatg |
| | | accTcaatgggcagactctcctcagcagctgggcaggcactg |
| | | tcctcctggcactccactgaGtactcatactccttgttgtcccctct |
| | | gactctctctgcagagagtgtagcagctccgcAcgtcaccctt |
| | | gggggtcagaagagcctctgctgcttttagacactgaatgtcaaat |
| | | caGtactgattgtcgtcagccaccagcaggtgaaacgtccagaa |
| | | taattcttggcctcgcaTatagaaaggtcttatttttgggttctttct |
| | | ggtcctttaaaatatcagtggaccaaaTtccatcttcctttttgtga |
| | | agcagcaggagcgaatggcttagaacctcgcctcctttgTgac |
| | | aggtgtactggccagcatctccaaactctttgacttggatggtca |
| | | gggttttgccAgagcctaagacctcactgctctggtccaaggt |
| | | ccaggtgataccatcttcttcaggggTgtcacaggtgaggac |
| | | caccatttctccaggggcatccggataccaatccaattctacgA |
| | | cataaacatctttcttcagacccatatggccacgaggggagatg |
| | | ccagaaaaaccagGgaaaaccaagagatgaccaactgctggt |
| | | gacacatggtggcgaccggtagcgctaggtCatatgcaggag |
| | | ttgaggttactgtgagtagtgattaaagagagtgatagggaactc |
| | | ttGaacaagagatgcaatttatactgttaattctggaaaaatattat |
| | | gggggtgtcaaaatGtcccgggacaattgacgccttctgtatga |
| | | aacagtttttcctccacgccttctgtatgAaacagtttttcctccac |
| | | gccttctgtatgaaacagtttttcctccgtcgaggacaattGacg |
| | | ccttctgtatgaaacagtttttcctccacgccttctgtatgaaacag |
| | | tttttcctccacgccttctgtatgaaacagtttttcctcc |
| 30 | IL12-9F2- | Cgatggctccggtgcccgtcagtgggcagagcgcacatcgcc |
| | 28BBZ | cacagtccccgagaagtTggggggaggggtcggcaattgaac |
| | | cggtgcctagagaaggtggcgcggggtaaactggGaaagtga |
| | | tgtcgtgtactggctccgcctttttcccgagggtgggggaga |
| | | accgtatatAagtgcagtagtcgccgtgaacgttctttttcg |
| | | caacgggtttgccgccagaacacaggTgtcgtgacgcggatc |
| | | caggcctaagcttacgcgtcctagcgctaccggtcgccacca |
| | | tGgccttaccagtgaccgccttgctcctgccgctggccttgc |
| | | tgctccacgccgccaggcCggaggtgcagctggtgcagagcg |
| | | gcgccgaggtgaagaagcccggcgccagcgtgaagGtgagct |
| | | gcaaggccagcggctacaccttcagcgactacgagatgcact |
| | | gggtgcggcaGgcccccggccagggcctggagtggatgggcg |
| | | ccatccaccccggcagcggcgacaccgCctacaaccagcggt |
| | | tcaagggccgggtgaccatcaccgccgacaagagcaccagca |
| | | ccGcctacatggagctgagcagcctgcggagcgaggacaccg |
| | | ccgtgtactactgcgcccgGttctacagctacgcctactggg |
| | | gccagggcaccctggtgaccgtgagcgccggtggagGcggtt |
| | | caggcggaggtggttctggcggtggcggatcggacatcgtga |
| | | tgacccagaccCccctgagcctgcccgtgacccccggcgagc |
| | | ccgccagcatcagctgccggagcagccaGagcctggtgcaca |
| | | gcaacggcaacacctacctgcagtggtacctgcagaagcccg |
| | | gccAgagcccccagctgctgatctacaaggtgagcaaccggt |
| | | tcagcggcgtgcccgaccggTtcagcggcagcggcagcggca |
| | | ccgacttcaccctgaagatcagccgggtggaggccgaGgacg |
| | | tgggcgtgtactactgcagccagagcatctacgtgccctaca |
| | | ccttcggccaggGcaccaagctggagatcaaacgtaccacga |
| | | cgccagcgccgcgaccaccaacaccggcgCccaccatcgcgt |
| | | cgcagcccctgtccctgcgcccagaggcgtgccggccagcgg |
| | | cgggGggcgcagtgcacacgagggggctggacttcgcctgtg |
| | | atttttgggtgctggtggtggTtggtggagtcctggcttgct |
| | | atagcttgctagtaacagtggcctttattattttctggGtga |
| | | ggagtaagaggagcaggctcctgcacagtgactacatgaaca |
| | | tgactccccgccgCcccgggccaacccgcaagcattaccagc |
| | | cctatgccccaccacgcgacttcgcagcctAtcgctccaaac |
| | | ggggcagaaagaaactcctgtatatattcaaacaaccattta |
| | | tgagaCcagtacaaactactcaagaggaagatggctgtagct |
| | | gccgatttccagaagaagaagaAggaggatgtgaactgagag |
| | | tgaagttcagcaggagcgcagacgcccccgcgtaccagcAgg |
| | | gccagaaccagctctataacgagctcaatctaggacgaagag |
| | | aggagtacgatgttTtggacaagagacgtggccgggaccctg |
| | | agatggggggaaagccgcagagaaggaagaaCcctcaggaag |
| | | gcctgtacaatgaactgcagaaagataagatggcggaggcct |
| | | acagtgAgattgggatgaaaggcgagcgccggaggggcaagg |
| | | ggcacgatggcctttaccagggtCtcagtacagccaccaagg |
| | | acacctacgacgcccttcacatgcaggccctgccccctcgCt |
| | | aggtcgacaatcaacctctggattacaaaatttgtgaaagat |
| | | tgactggtattcttaActatgttgctccttttacgctatgtg |
| | | gatacgctgctttaatgccifigtatcatgctAttgcttccc |
| | | gtatggctttcattttctcctccttgtataaatcctggttgc |
| | | tgtctctTtatgaggagttgtggcccgttgtcaggcaacgtg |
| | | gcgtggtgtgcactgtgtttgctgAcgcaacccccactggtt |
| | | ggggcattgccaccacctgtcagctccttttccgggactacG |
| | | ctttccccctccctattgccacggcggaactcatcgccgcct |
| | | gccttgcccgctgctgGacaggggctcggctgttgggcactg |
| | | acaattccgtggtgttgtcggggaagctgacgtCctttccat |
| | | ggctgctcgcctgtgttgccacctggattctgcgcgggacgt |
| | | ccttctgcTacgtcccttcggccctcaatccagcggaccttc |
| | | cttcccgcggcctgctgccggctctGcggcctcttccgcgtc |
| | | ttcgccttcgccctcagacgagtcggatctccctttgggccg |
| | | Cctccccgcctggaattcgctagcctcgagctcacacaaaaa |
| | | accaacacacagatgtaAtgaaaataaagatattttattgcg |
| | | gccgctttaggaagcattcagatagctcatcactCtatcaat |
| | | agtcactgcccgaattctgaaagcatgaagaagtatgcagag |
| | | cttgattttAgttttataaaaatccggttcttcaagggagga |
| | | ffittgtggcacagtctcactgttgaAattcagggcctgcat |
| | | cagctcatcaataactgccagcatgttttgatctagaaagat |
| | | cTgcctcttaggatccatcagaagctttgcattcatggtctt |
| | | gaactccacctggtacatCttcaagtcttcataaatactact |
| | | aaggcacagggccatcataaaagaggtctttctggAggccag |
| | | gcaactcccattagttatgaaagaggtctctctggaatttag |
| | | gcaactctcaTtcttggttaattccaatggtaaacaggcctc |
| | | cactgtgctggttttatcttttgtgatAtcttcatgatcaat |
| | | ctcttcagaagtgcaagggtaaaattctagagtttgtctggc |
| | | ctTctggagcatgttgctgacggccctcagcaggttttggga |
| | | gtggtgaaggcatgggaacAttcctgggtctggagtggccac |
| | | ggggaggtttctagatccgccgccacccgacccaccAccgcc |
| | | cgagccaccgccaccactgcagggcacagatgcccattcgct |
| | | ccaagatgagcTatagtagcggtcctgggcccgcacgctaat |
| | | gctggcatttttgcggcagatgaccgtgGctgaggtcttgtc |
| | | cgtgaagactctatctttcttttctctcttgctcttgccctg |
| | | gacCtgaacgcagaatgtcagggagaagtaggaatgtggagt |
| | | actccaggtgtcagggtactCccagctgacctccacctgccg |
| | | agaattctttaatggcttcagctgcaagttcttgggtGggtc |
| | | aggtttgatgatgtccctgatgaagaagctgctggtgtagtt |
| | | ttcatacttgagCttgtgaacggcatccaccatgacctcaat |
| | | gggcagactctcctcagcagctgggcaggCactgtcctcctg |
| | | gcactccactgagtactcatactccttgttgtcccctctgac |
| | | tctcTctgcagagagtgtagcagctccgcacgtcaccccttg |
| | | ggggtcagaagagcctctgctGcttttgacactgaatgtcaa |
| | | atcagtactgattgtcgtcagccaccagcaggtgaaacGtcc |
| | | agaataattcttggcctcgcatcttagaaaggtcttattttt |
| | | gggttctactggTcattttaaaatatcagtggaccaaattcc |
| | | atcttcctttttgtgaagcagcaggagcgaAtggcttagaac |
| | | ctcgcctcctttgtgacaggtgtactggccagcatctccaaa |
| | | ctcttTgacttggatggtcagggttttgccagagcttaagac |
| | | ctcactgctctggtccaaggtcCaggtgataccatcctcttc |
| | | aggggtgtcacaggtgaggaccaccatttctccaggggcAtc |
| | | cggataccaatccaattctacgacataaacatctttcttcag |
| | | ttcccatatggccaCgaggggagatgccagaaaaaccaggga |
| | | aaaccaagagatgaccaactgctggtgacacAtggtggcgac |
| | | cggtagcgctaggtcatatgcaggagttgaggttactgtgag |
| | | tagtgaTtaaagagagtgatagggaactcttgaacaagagat |
| | | gcaatttatactgttaattctggAaaaatattatgggggtgt |
| | | caaaatgtcccgggacaattgacgccttctgtatgaaacaGt |
| | | ttttcctccacgccttctgtatgaaacagtttttcctccacg |
| | | ccttctgtatgaaacAgtttttcctccgtcgaggacaattga |
| | | cgccttctgtatgaaacagtttttcctccacgccttctgtat |
| | | gaaacagtttttcctccacgccttctgtatgaaacagttttt |
| | | cctcc |
| 31 | IL12-9F2-Z | Cgatggctccggtgcccgtcagtgggcagagcgcacatcgcc |
| | | cacagtccccgagaagttGgggggaggggtcggcaattgaac |
| | | cggtgcctagagaaggtggcgcggggtaaactgggaAagtga |
| | | tgtcgtgtactggctccgcctttttcccgagggtgggggaga |
| | | accgtatataagTgcagtagtcgccgtgaacgttctttttcg |
| | | caacgggtttgccgccagaacacaggtgtcGtgacgcggatc |
| | | caggcctaagcttacgcgtcctagcgctaccggtcgccacca |
| | | tggcctTaccagtgaccgccttgctcctgccgctggccttgc |
| | | tgctccacgccgccaggccggaggTgcagctggtgcagagcg |
| | | gcgccgaggtgaagaagcccggcgccagcgtgaaggtgagct |
| | | Gcaaggccagcggctacaccttcagcgactacgagatgcact |
| | | gggtgcggcaggcccccgGccagggcctggagtggatgggcg |
| | | ccatccaccccggcagcggcgacaccgcctacaaccAgcggt |
| | | tcaagggccgggtgaccatcaccgccgacaagagcaccagca |
| | | ccgcctacatggAgctgagcagcctgcggagcgaggacaccg |
| | | ccgtgtactactgcgcccggttctacagctAcgcctactggg |
| | | gccagggcaccctggtgaccgtgagcgccggtggaggcggtt |
| | | caggcgGaggtggttctggcggtggcggatcggacatcgtga |
| | | tgacccagacccccctgagcctgcCcgtgacccccggcgagc |
| | | ccgccagcatcagctgccggagcagccagagcctggtgcaca |
| | | Gcaacggcaacacctacctgcagtggtacctgcagaagcccg |
| | | gccagagcccccagctgcTgatctacaaggtgagcaaccggt |
| | | tcagcggcgtgcccgaccggttcagcggcagcggcaGcggca |
| | | ccgacttcaccctgaagatcagccgggtggaggccgaggacg |
| | | tgggcgtgtactActgcagccagagcatctacgtgccctaca |
| | | ccttcggccagggcaccaagctggagatcaAacgtaccacga |
| | | cgccagcgccgcgaccaccaacaccggcgcccaccatcgcgt |
| | | cgcagcCcctgtccctgcgcccagaggcgtgccggccagcgg |
| | | cggggggcgcagtgcacacgagggGgctggacttcgcctgtg |
| | | atttttgggtgctggtggtggttggtggagtcctggcttgct |
| | | Atagcttgctagtaacagtggcctttattattttctgggtga |
| | | gagtgaagttcagcaggaGcgcagacgcccccgcgtaccagc |
| | | agggccagaaccagctctataacgagctcaatctagGacgaa |
| | | gagaggagtacgatgttttggacaagagacgtggccgggacc |
| | | ctgagatgggggGaaagccgcagagaaggaagaaccctcagg |
| | | aaggcctgtacaatgaactgcagaaagataAgatggcggagg |
| | | cctacagtgagattgggatgaaaggcgagcgccggaggggca |
| | | aggggcAcgatggcctttaccagggtctcagtacagccacca |
| | | aggacacctacgacgcccttcacaTgcaggccctgccccctc |
| | | gctaggtcgacaatcaacctctggattacaaaatttgtgaaa |
| | | Gattgactggtattcttaactatgttgctcattttacgctat |
| | | gtggatacgctgctttaaTgcctttgtatcatgctattgctt |
| | | cccgtatggctttcattttctcctccttgtataaatCctggt |
| | | tgctgtctctttatgaggagttgtggcccgttgtcaggcaac |
| | | gtggcgtggtgtGcactgtgtttgctgacgcaacccccactg |
| | | gttggggcattgccaccacctgtcagctccTttccgggactt |
| | | tcgctttccccctccctattgccacggcggaactcatcgccg |
| | | cctgccTtgcccgctgctggacaggggctcggctgttgggca |
| | | ctgacaattccgtggtgttgtcggGgaagctgacgtcctttc |
| | | catggctgctcgcctgtgttgccacctggattctgcgcggga |
| | | Cgtccttctgctacgtcccttcggccctcaatccagcggacc |
| | | ttccttcccgcggcctgcTgccggctctgcggcctcttccgc |
| | | gtcttcgccttcgccctcagacgagtcggatctcccTttggg |
| | | ccgcctccccgcctggaattcgctagcctcgagctcacacaa |
| | | aaaaccaacacaCagatgtaatgaaaataaagatattttatt |
| | | gcggccgctttaggaagcattcagatagctCatcactctatc |
| | | aatagtcactgcccgaattctgaaagcatgaagaagtatgca |
| | | gagcttGattttagttttataaaaatccggttcttcaaggga |
| | | ggatttttgtggcacagtctcactGttgaaattcagggcctg |
| | | catcagctcatcaataactgccagcatgttttgatctagaaa |
| | | Gatctgcctcttaggatccatcagaagctttgcattcatggt |
| | | cttgaactccacctggtaCatcttcaagtcttcataaatact |
| | | actaaggcacagggccatcataaaagaggtctttctGgaggc |
| | | caggcaactcccattagttatgaaagaggtctctctggaatt |
| | | taggcaactctcAttcttggttaattccaatggtaaacaggc |
| | | ctccactgtgctggttttatcttttgtgatAtcttcatgatc |
| | | aatctcttcagaagtgcaagggtaaaattctagagtttgtct |
| | | ggccttCtggagcatgttgctgacggccctcagcaggttttg |
| | | ggagtggtgaaggcatgggaacatTcctgggtctggagtggc |
| | | cacggggaggtttctagatccgccgccacccgacccaccacc |
| | | Gcccgagccaccgccaccactgcagggcacagatgcccattc |
| | | gctccaagatgagctataGtagcggtcctgggcccgcacgct |
| | | aatgctggcatttttgcggcagatgaccgtggctgaGgtctt |
| | | gtccgtgaagactctatctttcttttctctcttgctcttgcc |
| | | ctggacctgaacGcagaatgtcagggagaagtaggaatgtgg |
| | | agtactccaggtgtcagggtactcccagctGacctccacctg |
| | | ccgagaattctttaatggcttcagctgcaagttcttgggtgg |
| | | gtcaggTttgatgatgtccctgatgaagaagctgctggtgta |
| | | gttttcatacttgagcttgtgaacGgcatccaccatgacctc |
| | | aatgggcagactctcctcagcagctgggcaggcactgtcctc |
| | | Ctggcactccactgagtactcatactccttgttgtcccctct |
| | | gactctctctgcagagagTgtagcagctccgcacgtcacccc |
| | | ttgggggtcagaagagcctctgctgcttttgacactGaatgt |
| | | caaatcagtactgattgtcgtcagccaccagcaggtgaaacg |
| | | tccagaataattCttggcctcgcatcttagaaaggtcttatt |
| | | tttgggttctttctggtcctttaaaatatcAgtggaccaaat |
| | | tccatcttcctttttgtgaagcagcaggagcgaatggcttag |
| | | aacctcGcctccffigtgacaggtgtactggccagcatctcc |
| | | aaactctttgacttggatggtcagGgttttgccagagcctaa |
| | | gacctcactgctctggtccaaggtccaggtgataccatcttc |
| | | Ttcaggggtgtcacaggtgaggaccaccatttctccaggggc |
| | | atccggataccaatccaaTtctacgacataaacatctttctt |
| | | cagttcccatatggccacgaggggagatgccagaaaAaccag |
| | | ggaaaaccaagagatgaccaactgctggtgacacatggtggc |
| | | gaccggtagcgcTaggtcatatgcaggagttgaggttactgt |
| | | gagtagtgattaaagagagtgatagggaacTcttgaacaaga |
| | | gatgcaatttatactgttaattctggaaaaatattatggggg |
| | | tgtcaaAatgtcccgggacaattgacgccttctgtatgaaac |
| | | agtttttcctccacgccttctgtaTgaaacagtttttcctcc |
| | | acgccactgtatgaaacagtttttacctccgtcgaggacaat |
| | | Tgacgccttctgtatgaaacagtttttacctccacgccactg |
| | | Tatgaaacagtttttacctccacgccttctgtatgaaacaga |
| | | tttcctcc |
| 32 | mouse IL12- | Atggcctcaccgttgacccgctttctgtcgctgaacctgctg |
| | 9F2-Z | ctgctgggtgagtcgattAtcctggggagtggagaagctgag |
| | | gtgcagctggtgcagagcggcgccgaggtgaagaagCccggc |
| | | gccagcgtgaaggtgagctgcaaggccagcggctacaccttc |
| | | agcgactacgagAtgcactgggtgcggcaggcccccggccag |
| | | ggcctggagtggatgggcgccatccaccccGgcagcggcgac |
| | | accgcctacaaccagcggttcaagggccgggtgaccatcacc |
| | | gccgacAagagcaccagcaccgcctacatggagctgagcagc |
| | | ctgcggagcgaggacaccgccgtgTactactgcgcccggttc |
| | | tacagctacgcctactggggccagggcaccctggtgaccgtg |
| | | Agcgccggtggaggcggttcaggcggaggtggttctggcggt |
| | | ggcggatcggacatcgtgAtgacccagacccccctgagcctg |
| | | cccgtgacccccggcgagcccgccagcatcagctgcCggagc |
| | | agccagagcctggtgcacagcaacggcaacacctacctgcag |
| | | tggtacctgcagAagcccggccagagcccccagctgctgatc |
| | | tacaaggtgagcaaccggttcagcggcgtgCccgaccggttc |
| | | agcggcagcggcagcggcaccgacttcaccctgaagatcagc |
| | | cgggtgGaggccgaggacgtgggcgtgtactactgcagccag |
| | | agcatctacgtgccctacaccttcGgccagggcaccaagctg |
| | | gagatcaaacgtactactaccaagccagtgctgcgaactccc |
| | | Tcacctgtgcaccctaccgggacatctcagccccagagacca |
| | | gaagattgtcggccccgtGgctcagtgaaggggaccggattg |
| | | gacttcgcctgtgatatttacatctgggcacccttgGccgga |
| | | atctgcgtggcccttctgctgtccttgatcatcactctcatc |
| | | tgctaccacaggAgccgaagcaggagtgcagagactgctgcc |
| | | aacctgcaggaccccaaccagctctacaatGagctcaatcta |
| | | gggcgaagagaggaatatgacgtcttggagaagaagcgggct |
| | | cgggatCcagagatgggaggcaaacagcagaggaggaggaac |
| | | ccccaggaaggcgtatacaatgcaCtgcagaaagacaagatg |
| | | gcagaagcctacagtgagatcggcacaaaaggcgagaggcgg |
| | | Agaggcaaggggcacgatggcctttaccagggtctcagcact |
| | | gccaccaaggacacctatGatgccctgcatatgcagaccctg |
| | | gcctaggtcgactcacacaaaaaaccaacacacagaTgtaat |
| | | gaaaataaagatattttattcgtacgttaggcggagctcaga |
| | | tagcccatcaccCtgttgatggtcacgacgcgggtgctgaag |
| | | gcgtgaagcaggatgcagagcttcattttcActctgtaaggg |
| | | tctgcttctcccacaggaggtttctggcgcagagtctcgcca |
| | | ttatgaTtcagagactgcatcagctcatcgatggccaccagc |
| | | atgcccttgtctagaatgatctgcTgatggttgtgattctga |
| | | agtgctgcgttgatggcctggaactctgtctggtacatcttc |
| | | Aagtcctcatagatgctaccaaggcacagggtcatcatcaaa |
| | | gacgtcttctgtgggggcAggcagctccctcttgttgtggaa |
| | | gaagtctctctagtagccaggcaactctcgttcttgTgtagt |
| | | tccagtggtaaacaggtcttcaatgtgctggtttggtcccgt |
| | | gtgatgtcttcaTgatcgatgtcttcagcagtgcaggaataa |
| | | tgtttcagtttttctctggccgtcttcaccAtgtcatctgtg |
| | | gtcttcagcaggtttcgggactggctaagacacctggcaggt |
| | | ccagagActggaatgaccctagatccgccgccacccgaccca |
| | | ccaccgcccgagccaccgccaccgGatcggaccctgcaggga |
| | | acacatgcccacttgctgcatgaggaattgtaatagcgatcc |
| | | Tgagcttgcacgcagacattcccgcctttgcattggacttcg |
| | | gtagatgtcttctctacgAggaacgcacctttctggttacac |
| | | ccctcctctgtcctccttcatctttctttcttgcgcTggatt |
| | | cgaacaaagaacttgagggagaagtaggaatggggagtgctc |
| | | caggagtcagggTactcccagctgacctccacctgtgagttc |
| | | ttcaaaggcttcatctgcaagttcttgggcGggtctggtttg |
| | | atgatgtccctgatgaagaagctggtgctgtagttctcatat |
| | | ttattcTgctgccgtgcttccaacgccagttcaatgggcagg |
| | | gtctcctcggcagttgggcaggtgAcatcctcctggcaggac |
| | | actgaatactIctcatagtccctttggtccagtgtgaccttc |
| | | Tctgcagacagagacgccattccacatgtcactgcccgagag |
| | | tcaggggaactgctactgCtcttgatgttgaacttcaagtcc |
| | | atgtttctttgcaccagccatgagcacgtgaaccgtCcggag |
| | | taatttggtgcttcacacttcaggaaagtcttgtttttgaaa |
| | | ttttttaaaattTcagtggaccaaattccattttccttcttg |
| | | tggagcagcagatgtgagtggctcagagtcTcgcctcctttg |
| | | tggcaggtgtactggccagcatctagaaactctttgacagtg |
| | | atggtcAgggtctttccagagcctatgactccatgtctctgg |
| | | tctgaggtccaggtgatgtcatctTcttcaggcgtgtcacag |
| | | gtgaggttcactgtttctccaggggcatcgggagtccagtcc |
| | | Acctctacaacataaacgtctttctccagctcccacatggcc |
| | | atgagtggagacaccagcAaaacgatggcaaaccaggagatg |
| | | gttagcttctgaggacacatggtggcgaccggtagcGctagg |
| | | acgcgtaggacttgaggtcactgtgaggagtgattagcaagg |
| | | gtgataggcagcTcttcagcatgggaggcaatttatactgtt |
| | | aatgctggaaaaataatatgggggtgtcacGatgttcccggg |
| | | acaattgaacgccttctgtatgaaacaaattttcctcttaac |
| | | gccttcTgtatgaaacaaattttacctcttaacgccactgta |
| | | tgaaacaaattttcctcttaacgcCttctgtatgaaacaaat |
| | | tttcctcttaacgccttctgtatgaaacaaattttcctctta |
| | | acgccttctgtatgaaacaaattttcctctt |
| 33 | mouse IL12- | Atggcctcaccgttgacccgctttctgtcgctgaacctgctg |
| | 9F2-28Z | ctgctgggtgagtcgattAtcctggggagtggagaagctgag |
| | | gtgcagctggtgcagagcggcgccgaggtgaagaagCccggc |
| | | gccagcgtgaaggtgagctgcaaggccagcggctacaccttc |
| | | agcgactacgagAtgcactgggtgcggcaggcccccggccag |
| | | ggcctggagtggatgggcgccatccaccccGgcagcggcgac |
| | | accgcctacaaccagcggttcaagggccgggtgaccatcacc |
| | | gccgacAagagcaccagcaccgcctacatggagctgagcagc |
| | | ctgcggagcgaggacaccgccgtgTactactgcgcccggttc |
| | | tacagctacgcctactggggccagggcaccctggtgaccgtg |
| | | Agcgccggtggaggcggttcaggcggaggtggttctggcggt |
| | | ggcggatcggacatcgtgAtgacccagacccccctgagcctg |
| | | cccgtgacccccggcgagcccgccagcatcagctgcCggagc |
| | | agccagagcctggtgcacagcaacggcaacacctacctgcag |
| | | tggtacctgcagAagcccggccagagcccccagctgctgatc |
| | | tacaaggtgagcaaccggttcagcggcgtgCccgaccggttc |
| | | agcggcagcggcagcggcaccgacttcaccctgaagatcagc |
| | | cgggtgGaggccgaggacgtgggcgtgtactactgcagccag |
| | | agcatctacgtgccctacaccttcGgccagggcaccaagctg |
| | | gagatcaaacgtactactaccaagccagtgctgcgaactccc |
| | | Tcacctgtgcaccctaccgggacatctcagccccagagacca |
| | | gaagattgtcggccccgtGgctcagtgaaggggaccggattg |
| | | gacttcgcctgtgatatttacatctgggcacccttgGccgga |
| | | atctgcgtggcccttctgctgtccttgatcatcactctcatc |
| | | tgctaccacaggAgccgaaatagtagaaggaacagactcctt |
| | | caaagtgactacatgaacatgactccccggAggcctgggctc |
| | | actcgaaagccttaccagccctacgcccctgccagagacttt |
| | | gcagcgTaccgccccagcaggagtgcagagactgctgccaac |
| | | ctgcaggaccccaaccagctctacAatgagctcaatctaggg |
| | | cgaagagaggaatatgacgtcttggagaagaagcgggctcgg |
| | | Gatccagagatgggaggcaaacagcagaggaggaggaacccc |
| | | caggaaggcgtatacaatGcactgcagaaagacaagatggca |
| | | gaagcctacagtgagatcggcacaaaaggcgagaggCggaga |
| | | ggcaaggggcacgatggcctttaccagggtctcagcactgcc |
| | | accaaggacaccTatgatgccctgcatatgcagaccctggcc |
| | | taggtcgactcacacaaaaaaccaacacacAgatgtaatgaa |
| | | aataaagatattttattcgtacgttaggcggagctcagatag |
| | | cccatcAccctgttgatggtcacgacgcgggtgctgaaggcg |
| | | tgaagcaggatgcagagcttcattTtcactctgtaagggtct |
| | | gcttctcccacaggaggtttctggcgcagagtctcgccatta |
| | | Tgattcagagactgcatcagctcatcgatggccaccagcatg |
| | | cccttgtctagaatgatcTgctgatggttgtgattctgaagt |
| | | gctgcgttgatggcctggaactctgtctggtacatcTtcaag |
| | | tcctcatagatgctaccaaggcacagggtcatcatcaaagac |
| | | gtcttctgtgggGgcaggcagctccctcttgttgtggaagaa |
| | | gtctctctagtagccaggcaactctcgttcTtgtgtagttcc |
| | | agtggtaaacaggtcttcaatgtgctggtttggtcccgtgtg |
| | | atgtctTcatgatcgatgtcttcagcagtgcaggaataatgt |
| | | ttcagtttttctctggccgtcttcAccatgtcatctgtggtc |
| | | ttcagcaggtttcgggactggctaagacacctggcaggtcca |
| | | Gagactggaatgaccctagatccgccgccacccgacccacca |
| | | ccgcccgagccaccgccaCcggatcggaccctgcagggaaca |
| | | catgcccacttgctgcatgaggaattgtaatagcgaTcctga |
| | | gcttgcacgcagacattcccgcctttgcattggacttcggta |
| | | gatgtcttctctAcgaggaacgcacctttctggttacacccc |
| | | tcctctgtctccttcatcttttctttcttgCgctggattcga |
| | | acaaagaacttgagggagaagtaggaatggggagtgctccag |
| | | gagtcaGggtactcccagctgacctccacctgtgagttcttc |
| | | aaaggcttcatctgcaagttcttgGgcgggtctggtttgatg |
| | | atgtccctgatgaagaagctggtgctgtagttctcatattta |
| | | Ttctgctgccgtgcttccaacgccagttcaatgggcagggtc |
| | | tcctcggcagttgggcagGtgacatcctcctggcaggacact |
| | | gaatacttctcatagtccctttggtccagtgtgaccTtctct |
| | | gcagacagagacgccattccacatgtcactgcccgagagtca |
| | | ggggaactgctaCtgctcttgatgttgaacttcaagtccatg |
| | | tttctttgcaccagccatgagcacgtgaacCgtccggagtaa |
| | | tttggtgcttcacacttcaggaaagtcttgtttttgaaattt |
| | | tttaaaAtttcagtggaccaaattccattttccttcttgtgg |
| | | agcagcagatgtgagtggctcagaGtctcgcctccffigtgg |
| | | caggtgtactggccagcatctagaaactctttgacagtgatg |
| | | Gtcagggtctttccagagcctatgactccatgtctctggtct |
| | | gaggtccaggtgatgtcaTcttcttcaggcgtgtcacaggtg |
| | | aggttcactgtttctccaggggcatcgggagtccagTccacc |
| | | tctacaacataaacgtctttctccagctcccacatggccatg |
| | | agtggagacaccAgcaaaacgatggcaaaccaggagatggtt |
| | | agcttctgaggacacatggtggcgaccggtAgcgctaggacg |
| | | cgtaggacttgaggtcactgtgaggagtgattagcaagggtg |
| | | ataggcAgctcttcagcatgggaggcaatttatactgttaat |
| | | gctggaaaaataatatgggggtgtCacgatgttcccgggaca |
| | | attgaacgccttctgtatgaaacaaattttcctcttaacgcc |
| | | Ttctgtatgaaacaaattttcctcttaacgccttctgtatga |
| | | aacaaattttcctcttaaCgccttctgtatgaaacaaatttt |
| | | cctcttaacgccttctgtatgaaacaaattttcctcttaacg |
| | | ccttctgtatgaaacaaattacctctt |
| 34 | mouse IL12- | Atggcctcaccgttgacccgctttctgtcgctgaacctgctg |
| | 9F2-BBZ | ctgctgggtgagtcgattAtcctggggagtggagaagctgag |
| | | gtgcagctggtgcagagcggcgccgaggtgaagaagCccggc |
| | | gccagcgtgaaggtgagctgcaaggccagcggctacaccttc |
| | | agcgactacgagAtgcactgggtgcggcaggcccccggccag |
| | | ggcctggagtggatgggcgccatccaccccGgcagcggcgac |
| | | accgcctacaaccagcggttcaagggccgggtgaccatcacc |
| | | gccgacAagagcaccagcaccgcctacatggagctgagcagc |
| | | ctgcggagcgaggacaccgccgtgTactactgcgcccggttc |
| | | tacagctacgcctactggggccagggcaccctggtgaccgtg |
| | | Agcgccggtggaggcggttcaggcggaggtggttctggcggt |
| | | ggcggatcggacatcgtgAtgacccagacccccctgagcctg |
| | | cccgtgacccccggcgagcccgccagcatcagctgcCggagc |
| | | agccagagcctggtgcacagcaacggcaacacctacctgcag |
| | | tggtacctgcagAagcccggccagagcccccagctgctgatc |
| | | tacaaggtgagcaaccggttcagcggcgtgCccgaccggttc |
| | | agcggcagcggcagcggcaccgacttcaccctgaagatcagc |
| | | cgggtgGaggccgaggacgtgggcgtgtactactgcagccag |
| | | agcatctacgtgccctacaccttcGgccagggcaccaagctg |
| | | gagatcaaacgtactactaccaagccagtgctgcgaactccc |
| | | Tcacctgtgcaccctaccgggacatctcagccccagagacca |
| | | gaagattgtcggccccgtGgctcagtgaaggggaccggattg |
| | | gacttcgcctgtgatatttacatctgggcacccttgGccgga |
| | | atctgcgtggcccttctgctgtccttgatcatcactctcatc |
| | | tgctaccacaggAgccgaaaatggatcaggaaaaaattcccc |
| | | cacatattcaagcaaccatttaagaagaccActggagcagct |
| | | caagaggaagatgcttgtagctgccgatgtccacaggaagaa |
| | | gaaggaGgaggaggaggctatgagctgagcaggagtgcagag |
| | | actgctgccaacctgcaggaccccAaccagctctacaatgag |
| | | ctcaatctagggcgaagagaggaatatgacgtcttggagaag |
| | | Aagcgggctcgggatccagagatgggaggcaaacagcagagg |
| | | aggaggaacccccaggaaGgcgtatacaatgcactgcagaaa |
| | | gacaagatggcagaagcctacagtgagatcggcacaAaaggc |
| | | gagaggcggagaggcaaggggcacgatggcctttaccagggt |
| | | ctcagcactgccAccaaggacacctatgatgccctgcatatg |
| | | cagaccctggcctaggtcgactcacacaaaAaaccaacacac |
| | | agatgtaatgaaaataaagatattttattcgtacgttaggcg |
| | | gagctcAgatagcccatcaccctgttgatggtcacgacgcgg |
| | | gtgctgaaggcgtgaagcaggatgCagagcttcattttcact |
| | | ctgtaagggtctgcttctcccacaggaggtttctggcgcaga |
| | | Gtctcgccattatgattcagagactgcatcagctcatcgatg |
| | | gccaccagcatgcccttgTctagaatgatctgctgatggttg |
| | | tgattctgaagtgctgcgttgatggcctggaactctGtctgg |
| | | tacatcttcaagtcctcatagatgctaccaaggcacagggtc |
| | | atcatcaaagacGtcttctgtgggggcaggcagctccctctt |
| | | gttgtggttagaagtactctagtagccaggCaactctcgttc |
| | | ttgtgttcgttccagtggtaaacaggtatcaatgtgctggtt |
| | | tggtccCgtgtgatgtcttcatgatcgatgtcttcagcagtg |
| | | caggaataatgtttcagtttttctCtggccgtcttcaccatg |
| | | tcatctgtggtcttcagcaggtttcgggactggctaagacac |
| | | Ctggcaggtccagagactggaatgaccctagatccgccgcca |
| | | cccgacccaccaccgcccGagccaccgccaccggatcggacc |
| | | ctgcagggaacacatgcccacttgctgcatgaggaaTtgtaa |
| | | tagcgatcctgagcttgcacgcagacattcccgcctttgcat |
| | | tggacttcggtaGatgtcttctctacgaggaacgcacctttc |
| | | tggttacacccctcctctgtctccttcatcTtttctttcttg |
| | | cgctggattcgaacaaagaacttgagggagaagtaggaatgg |
| | | ggagtgCtccaggagtcagggtactcccagctgacctccacc |
| | | tgtgagttcttcaaaggcttcatcTgcaagttcttgggcggg |
| | | tctggtttgatgatgtccctgatgaagaagctggtgctgtag |
| | | Ttctcatatttattctgctgccgtgcttccaacgccagttca |
| | | atgggcagggtctcctcgGcagttgggcaggtgacatcctcc |
| | | tggcaggacactgaatacttctcatagtccctttggTccagt |
| | | gtgaccttctctgcagacagagacgccattccacatgtcact |
| | | gcccgagagtcaGgggaactgctactgctcttgatgttgaac |
| | | ttcaagtccatgtttctttgcaccagccatGagcacgtgaac |
| | | cgtccggagtaatttggtgcttcacacttcaggaaagtcttg |
| | | tttttgAaattttttaaaatttcagtggaccaaattccattt |
| | | tccttcttgtggagcagcagatgtGagtggctcagagtctcg |
| | | cctcctttgtggcaggtgtactggccagcatctagaaactct |
| | | Ttgacagtgatggtcagggtctttccagagcctatgactcca |
| | | tgtctctggtctgaggtcCaggtgatgtcatcttcttcaggc |
| | | gtgtcacaggtgaggttcactgtttctccaggggcaTcggga |
| | | gtccagtccacctctacaacataaacgtctttctccagctcc |
| | | cacatggccatgAgtggagacaccagcaaaacgatggcaaac |
| | | caggagatggttagcttctgaggacacatgGtggcgaccggt |
| | | agcgctaggacgcgtaggacttgaggtcactgtgaggagtga |
| | | ttagcaAgggtgataggcagctcttcagcatgggaggcaatt |
| | | tatactgttaatgctggaaaaataAtatgggggtgtcacgat |
| | | gttcccgggacaattgaacgccttctgtatgaaacaaatttt |
| | | Cctcttaacgccttctgtatgaaacaaattttcctcttaacg |
| | | ccttctgtatgaaacaaaTtttcctcttaacgccttctgtat |
| | | gaaacaaattttcctcttaacgccttctgtatgaaaCaaatt |
| | | ttcctcttaacgccttctgtatgaaacaaattttcctctt |
| 35 | mouse IL12- | Atggcctcaccgttgacccgctttctgtcgctgaacctgctg |
| | 9F2-28BBZ | ctgctgggtgagtcgattAtcctggggagtggagaagctgag |
| | | gtgcagctggtgcagagcggcgccgaggtgaagaagCccggc |
| | | gccagcgtgaaggtgagctgcaaggccagcggctacaccttc |
| | | agcgactacgagAtgcactgggtgcggcaggcccccggccag |
| | | ggcctggagtggatgggcgccatccaccccGgcagcggcgac |
| | | accgcctacaaccagcggttcaagggccgggtgaccatcacc |
| | | gccgacAagagcaccagcaccgcctacatggagctgagcagc |
| | | ctgcggagcgaggacaccgccgtgTactactgcgcccggttc |
| | | tacagctacgcctactggggccagggcaccctggtgaccgtg |
| | | Agcgccggtggaggcggttcaggcggaggtggttctggcggt |
| | | ggcggatcggacatcgtgAtgacccagacccccctgagcctg |
| | | cccgtgacccccggcgagcccgccagcatcagctgcCggagc |
| | | agccagagcctggtgcacagcaacggcaacacctacctgcag |
| | | tggtacctgcagAagcccggccagagcccccagctgctgatc |
| | | tacaaggtgagcaaccggttcagcggcgtgCccgaccggttc |
| | | agcggcagcggcagcggcaccgacttcaccctgaagatcagc |
| | | cgggtgGaggccgaggacgtgggcgtgtactactgcagccag |
| | | agcatctacgtgccctacaccttcGgccagggcaccaagctg |
| | | gagatcaaacgtactactaccaagccagtgctgcgaactccc |
| | | Tcacctgtgcaccctaccgggacatctcagccccagagacca |
| | | gaagattgtcggccccgtGgctcagtgaaggggaccggattg |
| | | gacttcgcctgtgatatttacatctgggcacccttgGccgga |
| | | atctgcgtggcccttctgctgtccttgatcatcactctcatc |
| | | tgctaccacaggAgccgaaatagtagaaggaacagactcctt |
| | | caaagtgactacatgaacatgactccccggAggcctgggctc |
| | | actcgaaagccttaccagccctacgcccctgccagagacttt |
| | | gcagcgTaccgccccaaatggatcaggaaaaaattcccccac |
| | | atattcaagcaaccatttaagaagAccactggagcagctcaa |
| | | gaggaagatgcttgtagctgccgatgtccacaggaagaagaa |
| | | Ggaggaggaggaggctatgagctgagcaggagtgcagagact |
| | | gctgccaacctgcaggacCccaaccagctctacaatgagctc |
| | | aatctagggcgaagagaggaatatgacgtcttggagAagaag |
| | | cgggctcgggatccagagatgggaggcaaacagcagaggagg |
| | | aggaacccccagGaaggcgtatacaatgcactgcagaaagac |
| | | aagatggcagaagcctacagtgagatcggcAcaaaaggcgag |
| | | aggcggagaggcaaggggcacgatggcctttaccagggtctc |
| | | agcactGccaccaaggacacctatgatgccctgcatatgcag |
| | | accctggcctaggtcgactcacacAaaaaaccaacacacaga |
| | | tgtaatgaaaataaagatattttattcgtacgttaggcggag |
| | | Ctcagatagcccatcaccctgttgatggtcacgacgcgggtg |
| | | ctgaaggcgtgaagcaggAtgcagagcttcattttcactctg |
| | | taagggtctgcttctcccacaggaggtttctggcgcAgagtc |
| | | tcgccattatgattcagagactgcatcagctcatcgatggcc |
| | | accagcatgcccTtgtctagaatgatctgctgatggttgtga |
| | | ttctgaagtgctgcgttgatggcctggaacTctgtctggtac |
| | | atcttcaagtcctcatagatgctaccaaggcacagggtcatc |
| | | atcaaaGacgtcttctgtgggggcaggcagctccctcttgtt |
| | | gtggaagaagtctctctagtagccAggcaactctcgttcttg |
| | | tgtagttccagtggtaaacaggtcttcaatgtgctggtttgg |
| | | Tcccgtgtgatgtcttcatgatcgatgtcttcagcagtgcag |
| | | gaataatgtttcagttttTctctggccgtcttcaccatgtca |
| | | tctgtggtcttcagcaggtttcgggactggctaagaCacctg |
| | | gcaggtccagagactggaatgaccctagatccgccgccaccc |
| | | gacccaccaccgCccgagccaccgccaccggatcggaccctg |
| | | cagggaacacatgcccacttgctgcatgagGaattgtaatag |
| | | cgatcctgagcttgcacgcagacattcccgcctttgcattgg |
| | | acttcgGtagatgtcttctctacgaggaacgcacctttctgg |
| | | ttacacccctcctctgtctccttcAtcttttctttcttgcgc |
| | | tggattcgaacaaagaacttgagggagaagtaggaatgggga |
| | | Gtgctccaggagtcagggtactcccagctgacctccacctgt |
| | | gagttcttcaaaggcttcAtctgcaagttcttgggcgggtct |
| | | ggtttgatgatgtccctgatgaagaagctggtgctgTagttc |
| | | tcatatttattctgctgccgtgcttccaacgccagttcaatg |
| | | ggcagggtctccTcggcagttgggcaggtgacatcctcctgg |
| | | caggacactgaatacttctcatagtcccttTggtccagtgtg |
| | | accttctctgcagacagagacgccattccacatgtcactgcc |
| | | cgagagTcaggggaactgctactgctcttgatgttgaacttc |
| | | aagtccatgtttctttgcaccagcCatgagcacgtgaaccgt |
| | | ccggagtaatttggtgcttcacacttcaggaaagtcttgttt |
| | | Ttgaaattttttaaaatttcagtggaccaaattccattttcc |
| | | ttcttgtggagcagcagaTgtgagtggctcagagtctcgcct |
| | | cctttgtggcaggtgtactggccagcatctagaaacTctttg |
| | | acagtgatggtcagggtctttccagagcctatgactccatgt |
| | | ctctggtctgagGtccaggtgatgtcatcttcttcaggcgtg |
| | | tcacaggtgaggttcactgtttctccagggGcatcgggagtc |
| | | cagtccacctctacaacataaacgtctttctccagctcccac |
| | | atggccAtgagtggagacaccagcaaaacgatggcaaaccag |
| | | gagatggttagcttctgaggacacAtggtggcgaccggtagc |
| | | gctaggacgcgtaggacttgaggtcactgtgaggagtgatta |
| | | Gcaagggtgataggcagctcttcagcatgggaggcaatttat |
| | | actgttaatgctggaaaaAtaatatgggggtgtcacgatgtt |
| | | cccgggacaattgaacgccttctgtatgaaacaaatTttcct |
| | | cttaacgccttctgtatgaaacaaattttcctcttaacgcct |
| | | tctgtatgaaacAaattttcctcttaacgccttctgtatgaa |
| | | acaaattttcctcttaacgccttctgtatgaaacaaattttc |
| | | ctcttaacgccttctgtatgaaacaaattttcctctt |

All documents mentioned in the present invention are cited as references in this application, as if each document is individually cited as a reference. In addition, it should be understood that after reading the above teaching contents of the present invention, a skilled person in the art can make various changes or modifications to the present invention, and these equivalent forms shall also fall within the scope defined by the appended claims of the present application.

1. An immune effector cell expressing a receptor and exogenous IL12, wherein the receptor specifically binds to GPC3.

2. The immune effector cell of claim 1, wherein the receptor and the IL12 are operably linked.

3. The immune effector cell of claim 1, wherein the immune effector cell includes: T cell, natural killer cell, cytotoxic T lymphocyte, natural killer T cell, DNT cell, and/or regulatory T cell.

4. The immune effector cell of claim 1, wherein the exogenous IL12 is constitutively or inductively expressed;
preferably, the promoter used to express the IL12 includes: an immune cell inducible promoter; and preferably, the immune cell inducible promoter is NFAT6 promoter.

5. The immune effector cell of any one of preceding claims, wherein the receptor has an extracellular domain specifically binding to GPC3, a transmembrane domain, and an intracellular signal domain.

6. The immune effector cell of claim 5, wherein the receptor is a chimeric antigen receptor, wherein the intracellular domain contains a cell stimulating signal molecule or a combination of a cell stimulating signal molecule and a cell activating co-stimulatory molecule;
Preferably, the cell stimulating signal molecule is selected from the functional signaling domain of a protein: CD3ζ, CD3γ, CD3δ, CD3ε, FcεRIγ, FcRβ, CD79a, CD79b, FcγRIIa, DAP10, or DAP12; more preferably CD3ζ; or
Preferably, the cell activating co-stimulatory molecule is selected from the functional signaling domain of the following proteins: CD27, CD28, CD137, CD134, ICOS, OX40, CD30, CD40, PD-1, lymphocyte function related antigen-1 (LFA-1), CD2, CD7, LIGHT, NKG2C, B7-H3, a ligand that specifically binds to CD83, CDS, ICAM-1, GITR, BAFFR, HVEM (LIGHTR), SLAMF7, NKp80 (KLRF1), CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, more preferably, CD27, CD28, CD137, CD134, ICOS.

7. The immune effector cell of claim 5 or 6, wherein the extracellular domain of the receptor contains an amino acid sequence that is at least 90%, 95%, 96%, 97%, 98%, or 99% identical with the amino acid sequence shown in SEQ ID NO: 25.

8. The immune effector cell of any one of preceding claims, wherein the receptor contains the amino acid sequence shown in SEQ ID NO: 21, 22, 23, or 24; or contains an amino acid sequence that is at least 90%, 95%, 96%, 97%, 98%, or 99% identical with the amino acid sequence shown in SEQ ID NO: 21, 22, 23 or 24.

9. The immune effector cell of claim 8, wherein the receptor and the exogenous IL12 are encoded by a nucleotide sequence that is at least 90%, 95%, 96%, 97%, 98%, or 99% identity with SEQ ID NO: 28, 29, 30, 31, 32, 33, 34 or 35;
preferably, encoded by a nucleotide sequence that is at least 90%, 95%, 96%, 97%, 98%, or 99% identity with SEQ ID NO: 28, 29, 30 or 31;
more preferably, encoded by a nucleotide sequence of SEQ ID NO: 28, 29, 30 or 31.

10. The immune effector cell of any one of preceding claims, wherein the immune effector cell does not contain exogenous co-stimulatory ligands.

11. The immune effector cell of any one of preceding claims, wherein the receptor and/or the IL12 are constitutively or inducibly expressed on the surface of the immune effector cell.

12. The immune effector cell of any one of preceding claims, wherein the immune effector cell contains an expression construct, which includes: an expression cassette of the antigen-binding receptor; and an expression cassette of the IL12.

13. The immune effector cell of any one of preceding claims, wherein the receptor and/or IL12 are expressed by using a viral vector; and preferably, the viral vector includes: a lentiviral vector, retroviral vector or adenoviral vector.

14. The immune effector cell of any one of claims 1-13, wherein after the immune effector cell is administered to an individual, the number of CAR-T cells in the peripheral blood of the individual is increased by at least 50%, compared with the case where the exogenous IL12 is not present.

15. The immune effector cell of any one of claims 1-13, wherein about 7 days after the immune effector cell is administered to the individual, the sum of the number of CAR-T cells in the individual's peripheral blood is greater than 6,000/μL; and about 10 days after the immune effector cell is administered, the sum of the number of CAR-T cells in the individual's peripheral blood is greater than 6,000/μL.

16. An expression construct, including an expression cassette of a receptor and an expression cassette of IL12, which are connected in sequence; wherein the antigen-binding receptor and IL12 are defined as in any one of preceding claims.

17. Use of the immune effector cell of any one of claims 1-13 in the preparation of a pharmaceutical composition for treating tumors in an individual in need thereof.

18. The use of claim 17, wherein the tumor includes: liver cancer, stomach cancer, lung cancer, breast cancer, head and neck cancer, bladder cancer, ovarian cancer, cervical cancer, kidney cancer, pancreatic cancer, cervical cancer, liposarcoma, melanoma, adrenal carcinoma, Schwannoma, malignant fibrous histiocytoma, esophageal cancer; and preferably, the tumor is liver cancer, gastric cancer, lung cancer, or breast cancer.

19. The use of claim 17 or 18, wherein the immune effector cells reduce the tumor by at least 50%.

20. The use of claim 19, wherein the immune effector cells reduce the tumor by at least 70%, and more preferably, the immune effector cells reduce the tumor by at least 80%.

21. A pharmaceutical composition, comprising:
the immune effector cell of any one of claims 1-13; and
a pharmaceutically acceptable carrier or excipient.

22. A kit comprising:
the immune effector cell of any one of claims 1-13; and
instructions on how to administer the immune effector cell to an individual.
